(19) United States
(12) Patent Application Publication
(10) Pub. No.: US 2016/0108381 A1
(43) Pub. Date: Apr. 21, 2016

(54) DNA POLYMERASES AND MUTANTS THEREOF

(71) Applicant: LIFE TECHNOLOGIES CORPORATION, Carlsbad, CA (US)

(72) Inventors: Jun LEE, San Diego, CA (US); Gary Gerard, Frederick, MD (US); Harini Shandilya, New Market, MD (US); Katherine Griffiths, Hornsby (AU); Moreland Gibbs, Sydney (AU); Peter Bergquist, Sydney (AU); Robert Potter, San Marcos, CA (US)

(21) Appl. No.: 14/875,576

(22) Filed: Oct. 5, 2015

Related U.S. Application Data
(63) Continuation of application No. 12/982,804, filed on Dec. 30, 2010.
Application No. 12/982,804 is a continuation of application No. 12/764,049, filed on Apr. 20, 2010, now abandoned.
Application No. 12/764,049 is a continuation of application No. 12/127,790, filed on May 27, 2008, now abandoned.
Application No. 12/127,790 is a continuation of application No. 10/244,081, filed on Sep. 16, 2002, now abandoned.
(60) Provisional application No. 60/318,903, filed on Sep. 14, 2001.

(51) Int. Cl. C12N 9/12; C12N 9/14; C12P 19/34

(52) U.S. Cl. CPC C12N 9/1252; C12P 19/34; C12N 9/14; C12Y 207/07007; C12Y 306/01

(57) ABSTRACT

The present invention provides polypeptides having a nucleotide polymerase activity and method of enhancing polymerase activity. The polypeptides of the present invention may possess both a DNA-dependent DNA polymerase activity and an RNA-dependent DNA polymerase activity, i.e., a reverse transcriptase activity. The polypeptides of the present invention may be used in any application including, but not limited to, DNA sequencing reactions, amplification reactions, cDNA synthesis reactions, and combined cDNA synthesis and amplification reactions, e.g., RT-PCR.

### CROSS REFERENCE TO RELATED APPLICATIONS

This application is a continuation of U.S. application Ser. No. 12/764,049 (pending), filed Apr. 20, 2010; which is a continuation of U.S. application Ser. No. 12/127,790 (abandoned), filed May 27, 2008; which is a continuation of U.S. application Ser. No. 10/244,081 (abandoned), filed Sep. 16, 2002; which claims priority to U.S. provisional patent application Ser. No. 60/318,903, filed Sep. 14, 2001, the disclosure of each of which is incorporated herein by reference in its entirety.

### BACKGROUND OF THE INVENTION

1. Field of the Invention

The present invention relates to the field of molecular biology. In particular, the present invention provides polypeptides having a nucleotide polymerase activity and method of enhancing polymerase activity. The polypeptides or polymerases of the present invention may posses both a DNA-dependent DNA polymerase activity and an RNA-dependent DNA polymerase activity, i.e., a reverse transcriptase (RT) activity. The polypeptides or polymerases of the present invention may be used in any application including, but not limited to, nucleic acid synthesis reactions, DNA sequencing reactions, amplification reactions, cDNA synthesis reactions, and combined cDNA synthesis and amplification reactions, e.g., RT-PCR.

2. Related Art

DNA polymerases synthesize formation of DNA molecules that are complementary to all or portion of nucleic acid templates. Upon hybridization of a primer to the single-stranded template, polymerases synthesize DNA in the 5′ to 3′ direction, i.e., successively adding nucleotides to the 3′-hydroxyl group of the growing strand. Thus, for example, in the presence of deoxyribonucleoside triphosphates (dNTPs) and a primer, a new DNA molecule, complementary to the single stranded nucleic acid template, can be synthesized. Typically an RNA or DNA template is used for synthesizing a complementary DNA molecule. However, other templates, such as chimeric templates or modified nucleic acid templates are also usable for synthesizing complementary molecules of polymerized nucleic acids. A DNA-dependent DNA polymerase utilizes a DNA template and produces a DNA molecule complementary to at least a portion of the template. An RNA-dependent DNA polymerase, i.e., a reverse transcriptase, utilizes an RNA template to produce a DNA strand complementary to at least a portion of the template, i.e., a cDNA. A common application of reverse transcriptases has been to transcribe mRNA into cDNA.

In addition to a polymerase activity, DNA polymerases may possess one or more additional catalytic activities. Typically, DNA polymerases may possess a 3′-5′ exonuclease activity and 5′-3′ exonuclease activity. Each of these activities has been localized to a particular region or domain of the protein. In E. coli Pol I, the N-terminal domain (amino acids 1-324) encodes the 5′-3′ exonuclease activity, the central domain (amino acids 324-517) encodes the 3′-5′ exonuclease activity and the C-terminal domain (amino acids 521-928) encodes the DNA polymerase activity. When E. coli Pol I is cleaved into two fragments by subtilisin digestion, the larger fragment (Klenow fragment) has 3′-5′ exonuclease and DNA polymerase activities and the smaller fragment has 5′-3′ exonuclease activity.

In addition to the E. coli polymerase discussed above, DNA polymerases have been isolated from a variety of mesophilic microorganisms. A number of these mesophilic DNA polymerases have also been cloned. Lin, et al. cloned and expressed T4 DNA polymerase in E. coli (Proc. Natl. Acad. Sci. USA 84:7000-7004 (1987)). Tabor, et al. (U.S. Pat. No. 4,795,699) describes a cloned T7 DNA polymerase, while Minkley, et al. (J. Biol. Chem. 259:10386-10392 (1984)) and Chatteree (U.S. Pat. No. 5,047,342) describe E. coli DNA polymerase I and the cloning of T5 DNA polymerase, respectively.

DNA polymerases have also been isolated and cloned from a variety of thermophilic organisms. These enzymes typically have a higher optimum temperature for polymerization activity than enzymes isolated from mesophilic organisms. Thermostable DNA polymerases have been discovered in a number of thermophilic organisms including, but not limited to Thermus aquaticus, Thermus thermophilus, and species of the Bacillus, Thermococcus, Sulfobus, and Pyrococcus genera. The thermostability of these enzymes has been exploited in numerous applications including the polymerase chain reaction (PCR).

The polymerase chain reaction (PCR) is used to amplify a target nucleic acid sequence from a sample. PCR utilizes denaturation of the target DNA, hybridization of oligonucleotide primers to specific sequences on opposite strands of the target DNA molecule, and subsequent extension of these primers with a DNA polymerase, usually a thermostable DNA polymerase, to generate two new strands of DNA which themselves can serve as templates for a further round of hybridization and extension. In PCR reactions, the product of one cycle serves as a template for the next cycle such that, at each repeat of the cycle, the amount of the specific sequence present in the reaction doubles. This leads to an exponential amplification process. If the polymerase employed is a thermostable enzyme, then fresh polymerase need not be added after every denaturation step because heat will not have destroyed the polymerase activity.

If the nucleotide sequence to be amplified by PCR is RNA, conventionally the nucleic acid molecule is first treated with reverse transcriptase in the presence of a primer to provide a cDNA template for amplification. In reverse transcription/polymerase chain reaction (RT-PCR), a DNA primer is hybridized to a strand of the target RNA molecule, and subsequent extension of this primer with a reverse transcriptase generates a new strand of DNA which can serve as a template for PCR. The preparation of the DNA molecule complementary to the template RNA molecule is referred to as the first strand reaction. Preparation of the DNA template is preferably carried out at an elevated temperature to avoid early termination of the reverse transcriptase reaction caused by RNA secondary structure. Unfortunately, the reverse transcriptase enzymes typically used have not been efficient at the desired elevated temperatures, e.g. above about 50° C. In addition, reverse transcriptase enzymes typically require reaction conditions that are not compatible with DNA-dependent DNA polymerases. This requires that the reaction conditions be manipulated after the first strand reaction in order to perform the subsequent amplification reaction, thereby adding substantially to the time and expense of the reaction and introducing a risk of contamination of the reaction mixture.

One approach that has been used to circumvent the necessity of manipulating the first strand reaction in an RT-PCR reaction has been to use a DNA polymerase alone and to modify the reaction conditions of the first strand reaction such that the DNA polymerase exhibits reverse transcriptase activity. This approach is demonstrated in U.S. Pat. Nos. 5,310,652, 5,322,770, 5,407,800, 5,561,058, 5,641,864, and 5,693,517. These patents disclose the use of Mn⁺² as a divalent cation to stimulate the reverse transcriptase activity of Taq polymerase. Although the presence of Mn⁺² stimulates RT activity, it also causes misincorporation of nucleotides by the DNA polymerase activity resulting in the introduction of errors into the amplified cDNA.

Thermostable DNA polymerase from Thermus aquaticus (Taq) made the polymerase chain reaction (PCR) feasible, and introduced a powerful technology that complemented recombinant DNA studies and aided in the diagnosis of inherited and infectious diseases (Innis, et al., (eds.) (1990) In PCR Protocols: A Guide to Methods and Applications. Academic Press, San Diego). Taq DNA polymerase also has reverse transcriptase activity (Jones and Foulkes, (1989) Nucleic Acids Res. 17, 8387-8388.). The reverse transcriptase activity of a recombinant DNA polymerase from Thermus thermophilus (rTth) (Myers and Gelfand, (1991) Biochem. 30, 7661-7666.) has been reported to be one hundred-fold greater than that of Taq DNA polymerase. The two enzymes have significant amino acid sequence similarity, and it is not clear why their abilities to utilize RNA templates are so different. Reverse transcription by thermophilic DNA polymerases has advantages over mesophilic retroviral reverse transcriptases (RTs) such as Moloney murine leukemia virus (M-MLV) and avian myeloblastosis virus (AMV) RT which are commonly used for cDNA synthesis, because the higher reaction temperatures with thermophilic polymerases help destabilize RNA secondary structures which typically pose problems for mesophilic RTs (DeStefano, et al., (1991). J. Biol. Chem. 266, 7423-7431.; Harrison, et al., (1998) Nucleic Acids Res. 26, 3433-3442.; Wu, et al., (1996) J. Virol. 70, 7132-7142.). The uses and advantages of using thermophilic DNA polymerases for reverse transcription and reverse transcription coupled PCR amplifications (RT-PCR) have been described (Myers and Gelfand, (1991)). However one of the disadvantages of using rTth DNA polymerase for copying RNA is the requirement for the use of Mn²⁺, rather than Mg²⁺, as divalent metal. The presence of Mn²⁺ results in a higher error rates during cDNA synthesis (Cadwell and Joyce, (1992) PCR Methods and Applications 2, 28-33.) and in reduced yields of DNA product during PCR amplification (Leung, et al., (1989) Technique 1, 11-15.). Special measures must be taken during the PCR step of RT-PCR to remove the influence of Mn²⁺ introduced during the reverse transcription step (Myer and Gelfand, (1991)).

Thus, there remains a need in the art for improved materials and methods for performing polymerization and/or reverse transcription reactions, e.g., RT-PCR reactions. This need and others are met by the present invention. The present invention provides a survey of a number of organisms including thermophilic bacteria to identify DNA polymerases that can be used to copy RNA efficiently at elevated temperatures and preferably in the presence of Mg²⁺ and/or salts thereof, as well as mutant DNA polymerases from other organisms that have gained advantageous properties such as having increased reverse transcriptase activity and/or having reverse transcriptase activity in the presence of Mg²⁺. The present invention provides DNA polymerase genes from such organisms. The DNA polymerases of the present invention preferably copy RNA efficiently in the presence of Mg²⁺. Their cloning, purification, and preliminary characterization are described.

### BRIEF SUMMARY OF THE INVENTION

In one aspect, the present invention provides polypeptides or polymerases that may have a DNA-dependent DNA polymerase activity and/or an RNA-dependent DNA polymerase activity, compositions and reaction mixtures comprising such polypeptides, nucleic acid molecules encoding such polypeptides (e.g., vectors), as well as host cells transformed with nucleic acid molecules encoding such polypeptides. In some embodiments, one or more of the activities of the polypeptides of the invention is thermostable. In some embodiments, both RNA-dependent and DNA-dependent DNA polymerase activities are thermostable. In some aspects, the polypeptides of the invention may be Pol I type DNA polymerases, which may be thermostable or mesophilic. In some embodiments, the polypeptide may be a DNA polymerase from a thermophilic eubacterium. The polypeptides of the invention may posses one or more additional activities, e.g., 5′-3′ exonuclease activity and/or 3′-5′ exonuclease activity. In some embodiments, the polypeptides may have reduced or substantially reduced 5′-3′ exonuclease activity and/or may have reduced or substantially reduced 3′-5′ exonuclease activity. In another aspect, polypeptides of the invention may lack or have an undetectable level of 5′-3′ exonuclease activity and/or 3′-5′ exonuclease activity.

In one aspect, polypeptides of the invention may be those having one or more nucleic acid polymerase activities (e.g., DNA-dependent DNA polymerase activity and/or RNA-dependent DNA polymerase activity) that may occur in the presence of Mg²⁺ or salts thereof (e.g., MgCl₂, MgSO₄, MgHPO₄, etc.). In a preferred aspect, both DNA-dependent DNA polymerase activity and RNA-dependent DNA polymerase activity may occur in the presence of Mg²⁺. In one aspect, nucleic acid polymerase activity may occur in the absence of Mn²⁺ or salts thereof. Thus, in one aspect, the present invention provides polypeptides having an RNA-dependent DNA polymerase activity (i.e., reverse transcriptase activity) that occurs in the presence of Mg²⁺ and does not require the presence of Mn²⁺. Polypeptides of the invention may have a specific activity level for RNA-dependent DNA polymerase activity in the presence of Mg²⁺ that is at least about 150, 250, 500, 750, 1,000, 2,000, 3,000, 4,000, 5,000, 7,500, 10,000, 25,000, 50,000, 75,000, 100,000, 150,000, 200,000, 250,000, 300,000, 400,000, or 500,000 units/mg protein. Thus, polypeptides of the invention may have a specific activity for RNA-dependent DNA polymerase activity of from about 150 to about 500,000, from about 150 to about 400,000, from about 150 to about 300,000, from about 150 to about 200,000, from about 150 to about 150,000, from about 150 to about 100,000, from about 150 to about 75,000, from about 150 to about 50,000, from about 150 to about 25,000, from about 150 to about 10,000, from about 150 to about 5,000, from about 150 to about 2,500, from about 150 to about 1,000, from about 150 to about 500, from about 150 to about 250, from about 500 to about 500,000, from about 500 to about 250,000, from about 500 to about 150,000, from about 500 to about 100,000, from about 500 to about 50,000, from about 500 to about 40,000, from about 500 to about 30,000, from about 500 to about 25,000, from about 500 to about 20,000, from about 500 to about 15,000, from about 500 to about 10,000, from about 500 to about 5,000, from about 500 to about 4,000, from about 500 to about 3,000, from about 500 to about 2,500, from about 500 to about 2,000, from about 500 to about 1,500, from about 500 to about 1,000, from about 750 to about 500,000, from about 750 to about 250,000, from about 750 to about 150,000, from about 750 to about 100,000, from about 750 to about 50,000, from about 750 to about 40,000, from about 750 to about 30,000, from about 750 to about 25,000, from about 750 to about 20,000, from about 750 to about 15,000, from about 750 to about 10,000, from about 750 to about 5,000, from about 750 to about 2,500, from about 750 to about 1,000, from about 1,000 to about 25,000, from about 1,000 to about 10,000, from about 1,000 to about 5,000, from about 1,000 to about 4,000, from about 1,000 to about 2,500, from about 25,000 to about 500,000, from about 25,000 to about 250,000, from about 25,000 to about 100,000, from about 25,000 to about 50,000, from about 50,000 to about 500,000, from about 50,000 to about 250,000, from about 50,000 to about 100,000, from about 100,000 to about 500,000, from about 100,000 to about 400,000, from about 100,000 to about 300,000, from about 100,000 to about 250,000, from about 100,000 to about 200,000, or from about 100,000 to about 150,000 units/mg protein. Specific activity is preferably determined as described herein. In one aspect, one unit of RNA-dependent DNA polymerase activity is the amount of enzyme required to incorporate 10 nmoles of dNTPs into acid insoluble product in 30 min under assay conditions specified herein. Such assay conditions may include elevated temperatures, for example temperatures of about 45° C., 50° C., 55° C., 60° C., 62° C., 65° C., 68° C., 70° C., 72° C. or 75° C. or higher, even up to 80° C., 85° C., 95° C. or 100° C. Suitable assay conditions are described herein (e.g. in Example 1).

Polypeptides of the invention may have a specific activity level for DNA-dependent DNA polymerase activity in the presence of Mg²⁺ or salts thereof that is at least about 1,000, 5,000, 10,000, 25,000, 50,000, 75,000, 100,000, 125,000, 150,000, 175,000, 200,000, 300,000, or 500,000 units/mg protein. Thus, polypeptides of the invention may have a specific activity for DNA-dependent DNA polymerase activity of from about 1,000 to about 500,000, from about 1,000 to about 300,000, from about 1,000 to about 200,000, from about 1,000 to about 100,000, from about 5,000 to about 500,000, from about 5,000 to about 250,000, from about 5,000 to about 150,000, from about 5,000 to about 100,000, from about 5,000 to about 75,000, from about 5,000 to about 50,000, from about 5,000 to about 25,000, from about 5,000 to about 15,000, from about 10,000 to about 500,000, from about 10,000 to about 250,000, from about 10,000 to about 150,000, from about 10,000 to about 100,000, from about 10,000 to about 75,000, from about 10,000 to about 50,000, from about 10,000 to about 40,000, from about 10,000 to about 25,000, from about 50,000 to about 500,000, from about 100,000 to about 500,000, from about 150,000 to about 500,000, from about 250,000 to about 500,000, from about 50,000 to about 300,000 from about 100,000 to about 300,000, from about 150,000 to about 300,000, from about 250,000 to about 300,000, from about 300,000 to about 500,000, from about 350,000 to about 500,000 from about 400,000 to about 500,000, from about 450,000 to about 500,000, or from about 150,000 to about 250,000, units/mg protein. One unit of DNA-directed DNA polymerase activity is the amount of enzyme required to incorporate 10 nmoles of dNTPs into acid insoluble product in 30 min under assay conditions described herein. Such assay conditions may include elevated temperatures, for example, temperatures of about 45° C., 50° C., 55° C., 60° C., 62° C., 65° C., 68° C., 70° C., 72° C. or 75° C. or higher, even up to 80° C., 85° C., 95° C. or 100° C.

In some embodiments, the ratio of RNA-dependent DNA polymerase specific activity to the DNA-dependent specific activity of the polypeptides of the invention (RNA:DNA) may be from about 0.025 to about 1, from about 0.025 to about 0.75, from about 0.025 to about 0.5, from about 0.025 to about 0.4, from about 0.025 to about 0.3, from about 0.025 to about 0.25, from about 0.025 to about 0.2, from about 0.025 to about 0.15, from about 0.025 to about 0.1, from about 0.025 to about 0.05, from about 0.05 to about 1, from about 0.05 to about 0.75, from about 0.05 to about 0.5, from about 0.05 to about 0.4, from about 0.05 to about 0.3, from about 0.05 to about 0.25, from about 0.05 to about 0.2, from about 0.05 to about 0.15, from about 0.05 to about 0.1, from about 0.1 to about 1, from about 0.1 to about 0.75, from about 0.1 to about 0.5, from about 0.1 to about 0.4, from about 0.1 to about 0.3, from about 0.1 to about 0.25, from about 0.1 to about 0.2, or from about 0.1 to about 0.15 when both activities are determined as described herein. These ratios may be determined using assays performed at elevated temperatures, for example, temperatures of about 45° C., 50° C., 55° C., 60° C., 62° C., 65° C., 68° C., 70° C., 72° C. or 75° C. or higher, even up to 80° C., 85° C., 95° C. or 100° C. In some embodiments, the temperature used to determine the RNA-dependent DNA polymerase specific activity may be the same as the temperature used to determine the DNA-dependent DNA polymerase specific activity. In other embodiments, these temperatures may be different.

Polypeptides of the invention may have increased RNA-dependent DNA polymerase activity compared to other known DNA polymerases such as Tth DNA polymerase, Taq DNA polymerase or Tne DNA polymerase. In some aspects, the increase in RNA-dependent DNA polymerase activity for a polypeptide of the invention may be at least about 5%, 10%, 25%, 30%, 50%, 100%, 150%, 200%, 300%, 500%, 1,000%, 2,500%, or 5,000% compared to Tth DNA polymerase, Taq DNA polymerase and/or Tne DNA polymerase. The increase in RNA-dependent DNA polymerase activity may range from about 5% to about 5,000%, from about 5% to about 2,500%, from about 5% to about 1,000%, from about 5% to about 500%, from about 5% to about 250%, from about 5% to about 100%, from about 5% to about 50%, from about 5% to about 25%, from about 25% to about 5,000%, from about 25% to about 2,500%, from about 25% to about 1,000%, from about 25% to about 500%, from about 25% to about 250%, from about 25% to about 100%, from about 25% to about 50%, from about 100% to about 5,000%, from about 100% to about 2,500%, from about 100% to about 1,000%, from about 100% to about 500%, or from about 100% to about 250%. An increase in RNA-dependent DNA polymerase activity may also be measured by relative activity compared to Tth DNA polymerase, Taq DNA polymerase and/or Tne DNA polymerase. Preferably, the RNA-dependent DNA polymerase activity of the polyps of the invention is at least about 1.1, 1.2, 1.5, 2, 5, 10, 25, 50, 75, 100, 150, 200, 300, 500, 1,000, 2,500, 5,000, 10,000, or 25,000 fold higher than the RNA-dependent DNA polymerase activity of the Tth DNA polymerase, Taq DNA polymerase and/or Tne DNA polymerase. The increase in RNA-dependent DNA polymerase activity may range from about 1.1 fold to about 25,000 fold, from about 1.1 fold to about 10,000 fold, from about 1.1 fold to about 5,000 fold, from about 1.1 fold to about 2,500 fold, from about 1.1 fold to about 1,000 fold, from about 1.1 fold to about 500 fold, from about 1.1 fold to about 250 fold, from about 1.1 fold to about 100 fold, from about 1.1 fold to about 50 fold, from about 1.1 fold to about 25 fold, from about 1.1 fold to about 10 fold, from about 1.1 fold to about 5 fold, from about 5 fold to about 25,000 fold, from about 5 fold to about 5,000 fold, from about 5 fold to about 1,000 fold, from about 5 fold to about 500 fold, from about 5 fold to about 100 fold, from about 5 fold to about 50 fold, from about 5 fold to about 25 fold, from about 50 fold to about 25,000 fold, from about 50 fold to about 10,000 fold, from about 50 fold to about 5,000 fold, from about 50 fold to about 2,500 fold, from about 50 fold to about 1,000 fold, from about 50 fold to about 500 fold, from about 50 fold to about 250 fold, or from about 50 fold to about 100 fold. Preferably, such activities are determined under conditions described herein and then compared to calculate the fold increase in activity of the polypeptide of the invention relative to the Tth, Tne and/or Taq DNA polymerase. In one aspect, the activities are determined in the presence of Mg²⁺ and are preferably done under conditions (e.g., temperature, pH, ionic strength, etc.) which are optimum for the enzymes tested. Such conditions may include elevated temperatures, for example, temperatures from about 45° C. 50° C., 55° C., 60° C., 62° C., 65° C., 68° C., 70° C., 72° C., or 75° C. or higher, even up to 80° C., 85° C., 95° C., or 100° C.

Polypeptides of the invention may be isolated from organisms that naturally express them. Alternatively, nucleic acids encoding the polypeptides may be cloned and introduced into appropriate host cells. Polypeptides of the invention may also be prepared by mutating or modifying a nucleic acid molecule to encode a polymerase of the invention. Polypeptides according to this aspect of the invention may contain one or more motifs associated with Mg²⁺ dependent reverse transcriptase activity. Such motifs include, but are not limited to the Q-helix sequences associated with Mg²⁺ dependent activity and the presence of specified amino acid residues at positions identified herein. A representative Q-helix may have the sequence RY—X₈—Y—X₃—SFAER, (SEQ ID NO:1) wherein X is any imino or amino acid. Other representative Q-helices (see Tables 35 and 37) include amino acid numbers 823 to 842 of the sequence of E. coli DNA polymerase I (SEQ ID NO:34), amino acid numbers 728 to 747 of Thermus aquaticus (Taq) DNA polymerase (SEQ ID NO:27), and amino acid numbers 820-838 of the Caldibacillus cellulovorans CompA.2 DNA polymerase amino acid sequence (SEQ ID NO:16). Each X may independently represent an Ala, Cys, Asp, Glu, Phe, Gly, His, Ile, Lys, Leu, Met, Asn, Pro, Gln, Arg, Ser, Thr, Val, Trp, or Tyr or may represent an amino or imino acid that is not naturally produced in most host cells. Q-helix motifs associated with Mg²⁺ dependent activity include, but are not limited to, Q-helices wherein position 11 of the Q-helix (SEQ ID NO:1) may be a phenylalanine or a tyrosine (F or Y) independently of the amino acid residue at positions 15 and/or 16. In some embodiments, position 15 of the Q-helix (SEQ ID NO:1) may be a serine or asparagine (S or N) independently of the amino acid residue at positions 11 and/or 16. In some embodiments, position 16 of the Q-helix (SEQ ID NO:1) may be a tyrosine or phenylalanine (Y or F) independently of the amino acid residue at positions 11 and/or 15. In one embodiment, position 11 may be a phenylalanine residue while position 15 is a serine residue and position 16 is a phenylalanine. In another embodiment, position 11 may be tyrosine, while position 15 may be serine, and position 16 may be phenylalanine.

In another aspect, polypeptides of the invention include those with one or more specified amino acid residues at positions that correspond to Q628, 1659, Q668, F669 and/or Q753 of the Caldibacillus cellulovorans CompA.2 (CompA.2) DNA polymerase amino acid sequence of SEQ ID NO:16. In some embodiments, polypeptides of the invention may include a residue at a position that corresponds to position Q628 that is not a lysine or glutamate residue. Suitable amino acid residues include Ala, Cys, Asp, Phe, Gly, His, Ile, Leu, Met, Asn, Pro, Gln, Arg, Ser, Thr, Val, Trp, or Tyr. In some embodiments, polypeptides of the invention may have a glutamine residue at a position corresponding to position Q628 of the CompA.2 polymerase. In some embodiments, polypeptides of the invention may include a residue at a position corresponding to 1659 of the CompA.2 DNA polymerase that is not a glycine. Suitable residues include Ala, Cys, Asp, Glu, Phe, His, Ile, Lys, Leu, Met, Asn, Pro, Gln, Arg, Ser, Thr, Val, Trp, or Tyr or may be an amino or imino acid that is not naturally produced in most host cells. In some embodiments, polypeptides of the invention may have a hydrophobic residue at this position, for example, Ile, Val, and/or Leu. In some embodiments, polypeptides of the invention may include a residue at a position corresponding to Q668 of the CompA.2 DNA polymerase that is not a serine. Suitable residues include Ala, Cys, Asp, Glu, Phe, Gly, His, Ile, Lys, Leu, Met, Asn, Pro, Gln, Arg, Thr, Val, Trp, or Tyr or may be an amino or imino acid that is not naturally produced in most host cells. In some embodiments, polypeptides of the invention may have a glutamine and/or a threonine at this position. In some embodiments, polypeptides of the invention may include a residue at a position corresponding to F669 of the CompA.2 DNA polymerase that is not an aspartate or glutamate. Suitable residues include Ala, Cys, Phe, Gly, His, Ile, Lys, Leu, Met, Asn, Pro, Gln, Arg, Ser, Thr, Val, Trp, or Tyr or may be an amino or imino acid that is not naturally produced in most host cells. In some embodiments, polypeptides of the invention may have an aromatic amino acid at this position, for example, a phenylalanine. In some embodiments, polypeptides of the invention may include a residue at a position corresponding to Q753 of the CompA.2 DNA polymerase that is not an alanine or valine. Suitable residues include Cys, Asp, Glu, Phe, Gly, His, Ile, Lys, Leu, Met, Asn, Pro, Gln, Arg, Ser, Thr, Trp, or Tyr or may be an amino or imino acid that is not naturally produced in most host cells. In some embodiments, polypeptides of the invention may have a glutamine at this position.

In one aspect, the present invention provides polypeptides having nucleic acid polymerase activity that may be isolated and/or cloned from a organism of interest (e.g., a eukaryotic cell, a prokaryotic cell, a virus, etc.). Suitable organisms include, but are not limited to, archaeabacteria and eubacteria.

In some embodiments, a polypeptide of the invention may be isolated, or a nucleic acid encoding such a polypeptide may be cloned from one or more eubacteria including, but not limited to, Clostridium spp. (e.g., Clostridium stercorarium, Clostridium thermosulfurogenes, etc.), Caldibacillus spp. (e.g., Caldibacillus cellulovorans CompA.2), Caldicellulosiruptor spp. (e.g., Caldicellulosiruptor Tok13B, Caldicellulosiruptor Tok7B, Caldicellulosiruptor RT69B), Bacillus spp. (e.g., Bacillus caldolyticus EA1), Thermus spp. (e.g., Thermus RT41A), Dictyoglomus spp. (e.g., Dictyoglomus thermophilum), Spirochaete spp., and Tepidomonas spp.

In some aspects, the polypeptides of the invention include PolI type DNA polymerases, which may be thermophilic or mesophilic. In other aspects, the polypeptides of the invention include Pol III type DNA polymerases, which may be thermophilic or mesophilic.

The present invention also relates to fragments and mutants of the polypeptides of the invention that may possess one or more desirable characteristics (e.g., enzymatic activity, antigenicity, etc.). In some embodiments, the mutants and fragments of the polypeptides of the invention may possess a polymerase activity including a RNA-dependent DNA polymerase activity and/or a DNA-dependent DNA polymerase activity. The present invention also includes fragments of mutants of the polypeptides of the invention. Mutants, fragments and/or fragments of mutants may comprise one or more activities associated with the corresponding un-mutated or wild type polypeptide (such as 5′-3′ exonuclease activity, 3′-5′ exonuclease activity, etc.) or may have decreased activity (e.g., decreased 5′-3′ exonuclease activity and/or decreased 3′-5′ exonuclease activity, etc.) and/or increased activity (e.g., increase RNA-dependent DNA polymerase activity, increase DNA-dependent DNA polymerase activity, and/or increase thermostability, etc.) compared to the un-mutated or wildtype polypeptide. In some embodiments, polypeptides of the invention include mutants and/or fragments of DNA polymerases from one or more the organisms listed above. In some embodiments, mutants, fragments, and/or fragments of mutants may be isolated from, or nucleic acid encoding them may be cloned from, thermophilic eubacteria including, but not limited to Clostridium spp. (e.g., Clostridium stercorarium, Clostridium thermosulfurogenes, etc.), Caldibacillus spp. (e.g., Caldibacillus cellulovorans CompA.2), Caldicellulosiruptor spp. (e.g., Caldicellulosiruptor Tok13B, Caldicellulosiruptor Tok7B, Caldicellulosiruptor RT69B), Bacillus spp. (e.g., Bacillus caldolyticus EA1), Thermus spp. (e.g., Thermus RT41A), Dictyoglomus spp. (e.g., Dictyoglomus thermophilum) Spirochaete spp., and Tepidomonas spp.

In another aspect, polypeptides of the invention include polypeptides having one or more mutations and/or deletions that increase/decrease one or more desirable/undesirable characteristic of the polypeptide. For example, the present invention provides polypeptides with mutations that result in enhanced RNA-dependent DNA polymerase activity, enhanced thermostability of the RNA-dependent and/or DNA-dependent polymerase activity of the polypeptide; mutations that result in the ability or improved ability of the mutant polypeptide to, under selected conditions, incorporate dideoxynucleotides into a DNA molecule; mutations that decrease exonuclease activity and the like as compared to the non-mutated wildtype polypeptide. In some embodiments, polypeptides of the invention may comprise one or more mutations that enhance the RNA-dependent DNA polymerase activity of the polypeptide as compared to the non-mutated, wild type polypeptide. In particular, mutations may confer upon polypeptides of the invention the ability perform RNA-dependent DNA polymerase activity in the presence of Mg²⁺ and, optionally, in the absence of Mn²⁺ and/or may increase ability of polypeptides of the invention to perform RNA-dependent DNA polymerase activity in the presence of Mg²⁺ and, optionally, in the absence of Mn²⁺.

In some embodiments, the present invention provides mutant or modified DNA polymerases. Such mutants or modified polymerases may be prepared from any DNA polymerase (e.g., bacterial, viral, and/or eukaryotic polymerases). Such DNA polymerases may include Pol I type or Pol III type DNA polymerases, which may be thermophilic or mesophilic. Preferably, such mutants may have an increased RNA-dependent DNA polymerase activity as compared to the corresponding wildtype or unmutated or unmodified polymerase (e.g., in the presence of Mg²⁺ and/or in the absence of Mn²⁺). In some embodiments, mutant polypeptides of the invention may have one or more mutations or modifications that result in one or more amino acid changes (which may include addition of amino acids, substitutions of amino acids and/or deletions of amino acids or combinations thereof) in the Q-helix which increases the RNA-dependent DNA polymerase activity of the mutant or modified enzyme compared to the wild type or unmutated or unmodified enzyme. One skilled in the art can readily determine the corresponding Q-helix for any DNA polymerase by using standard sequence alignment techniques comparing the sequences of the polymerase of interest to the Q-helix sequences identified herein. A representative Q-helix is defined as RY—X₈—Y—X₃—SFAER (SEQ ID NO:1), wherein X is any imino or amino acid. Representative Q-helices (see Tables 35 and 37) include amino acid numbers 823 to 842 (SEQ ID NO:65) of the sequence of E. coli DNA polymerase I, amino acid numbers 728 to 747 (SEQ ID NO:46) of the sequence of Thermus aquaticus (Taq) DNA polymerase, and amino acid numbers 820-838 of the Caldibacillus cellulovorans CompA.2 DNA polymerase amino acid sequence of SEQ ID NO:16. Each X may independently represent an Ala, Cys, Asp, Glu, Phe, Gly, His, Ile, Lys, Leu, Met, Asn, Pro, Gln, Arg, Ser, Thr, Val, Trp, or Tyr or may represent an amino or imino acid that is not naturally produced in most host cells. Each X can be determined by selecting a corresponding nucleic acid codon. Modified or natural tRNAs can be used to introduce specific amino acids into the sequence at any position. Once the Q-helix is identified for a polymerase of interest, any number of modifications or mutations can be made (e.g., deletions, point mutations, insertions etc.) which preferably change the amino acid sequence and then the resulting mutant or modified polymerase can be assayed to determine the effect of the mutation or modification. Preferably, such mutations or modifications are designed based on the sequences found in one or more of the polypeptides of the invention. In some preferred embodiments, a polypeptide of the invention may have a mutation at position 11 of the Q-helix (SEQ ID NO:1). Such a mutation may preferably change an amino acid to a phenylalanine or a tyrosine (F or Y) independently of the amino acid residue at positions 15 and/or 16 of the Q-helix. In some embodiments, mutants of the invention may have a mutation at position 15 of the Q-helix. Such a mutation may change an amino acid at this position to a serine or asparagine (S or N) independently of the amino acid residue at positions 11 and/or 16. In some embodiments, polypeptides of the invention may possess a mutation at position 16 of the Q-helix. Such a mutation may change an amino acid to be a tyrosine or phenylalanine (Y or F) independently of the amino acid residue at positions 11 and/or 15. In some embodiments, polypeptides of the invention may possess multiple mutations, for example, at positions 11, 15, and 16, or at two of these three positions. In one embodiment, position 11 may be a phenylalanine residue while position 15 is a serine residue and position 16 is a phenylalanine. In another embodiment, position 11 may be tyrosine, while position 15 may be serine, and position 16 may be phenylalanine.

In another aspect, mutant or modified polypeptides of the invention include those with one or more mutations or modifications in amino acid residues at positions that correspond to Q628, 1659, Q668, F669 and/or Q753 of Caldibacillus cellulovorans CompA.2 (CompA.2) DNA polymerase amino acid sequence SEQ ID NO:16. Such mutations preferably result in an increase in the RNA-dependent DNA polymerase activity of the mutant as compared to the wildtype or unmutated or unmodified enzyme. In some embodiments, mutant polypeptides of the invention may include a mutation of a residue at a position that corresponds to position Q628 of the CompA.2 DNA polymerase. Such a mutation preferably changes the amino acid at this position to a residue that is not a lysine or glutamate residue. Suitable amino acid residues include Ala, Cys, Asp, Phe, Gly, His, Ile, Leu, Met, Asn, Pro, Gln, Arg, Ser, Thr, Val, Trp, or Tyr. In some embodiments, mutant polypeptides of the invention may be mutated to have a glutamine residue at a position corresponding to position Q628 of the CompA.2 polymerase. In some embodiments, mutant polypeptides of the invention may mutated to include a residue at a position corresponding to 1659 of the CompA.2 DNA polymerase that is not a glycine. Suitable residues include Ala, Cys, Asp, Glu, Phe, His, Ile, Lys, Leu, Met, Asn, Pro, Gln, Arg, Ser, Thr, Val, Trp, or Tyr or may be an amino or imino acid that is not naturally produced in most host cells. In some embodiments, polypeptides of the invention may be mutated to have a hydrophobic residue at this position, for example, Ile, Val, and/or Leu. In some embodiments, mutant polypeptides of the invention may be mutated to include a residue at a position corresponding to Q668 of the CompA.2 DNA polymerase that is not a serine. Suitable residues include Ala, Cys, Asp, Glu, Phe, Gly, His, Ile, Lys, Leu, Met, Asn, Pro, Gln, Arg, Thr, Val, Trp, or Tyr or may be an amino or imino acid that is not naturally produced in most host cells. In some embodiments, mutant polypeptides of the invention may be mutated to have a glutamine and/or a threonine at this position. In some embodiments, mutant polypeptides of the invention may be mutated to include a residue at a position corresponding to F669 of the CompA.2 DNA polymerase that is not an aspartate or glutamate. Suitable residues include Ala, Cys, Phe, Gly, His, Ile, Lys, Leu, Met, Asn, Pro, Gln, Arg, Ser, Thr, Val, Trp, or Tyr or may be an amino or imino acid that is not naturally produced in most host cells. In some embodiments, mutant polypeptides of the invention may be mutated to have an aromatic amino acid at this position, for example, a phenylalanine. In some embodiments, mutant polypeptides of the invention may be mutated to include a residue at a position corresponding to Q753 of the CompA.2 DNA polymerase that is not an alanine or valine. Suitable residues include Cys, Asp, Glu, Phe, Gly, His, Ile, Lys, Leu, Met, Asn, Pro, Gin, Arg, Ser, Thr, Trp, or Tyr or may be an amino or imino acid that is not naturally produced in most host cells. In some embodiments, polypeptides of the invention may be mutated to have a glutamine at this position.

In some embodiments, polymerases of the invention may comprise one or more mutations or modifications that enhance RNA-dependent DNA polymerase activity that are not located in the Q-helix (e.g., at positions Q628, 1659, Q668, F669 and/or Q753) and such mutations may be made alone or be made in conjunction with mutations in the Q-helix. One skilled in the art can identify corresponding amino acid residues in other DNA polymerases by similarly aligning one or more of the polypeptides of the invention (e.g., the Caldibacillus cellulovorans CompA.2 DNA polymerase) with one or more polymerases of interest. In some embodiments, one or more amino acid residues in a eubacterial DNA polymerase corresponding to one or more of the Caldibacillus cellulovorans CompA.2 DNA polymerase amino acid residues identified above, can be mutated to have all or a portion of the amino acid sequence present in the Caldibacillus cellulovorans CompA.2 DNA polymerase.

In one aspect, mutant or modified polypeptides of the invention may possess an increased RNA-dependent DNA polymerase activity compared to the corresponding unmutated or unmodified or wildtype polymerase or as compared to one or more prior art polymerases (e.g., Thermus thermophilus polymerase). In some embodiments, a polymerase having an increase in RNA-dependent DNA polymerase activity may be a mutated DNA polymerase that has at least about a 5% increase, 10% increase, 25% increase, 30% increase, 50% increase, 100% increase, 150% increase, 200% increase, 300%, 500% increase, 1,000% increase, 2,500% increase or 5,000% increase in the RNA-dependent DNA polymerase activity as compared to (1) the corresponding unmutated or wild-type enzyme; or (2) a particular polymerase (e.g., Thermus thermophilus (Tth) polymerase) or group of polymerases. Thus mutant polymerases of the invention may have an increase in RNA-dependent DNA polymerase activity of from about 5% to about 5,000%, from about 5% to about 2,500%, from about 5% to about 1000%, from about 5% to about 500%, from about 5% to about 250%, from about 5% to about 100%, from about 5% to about 50%, from about 5% to about 25%, from about 25% to about 5,000%, from about 25% to about 2,500%, from about 25% to about 1,000%, from about 25% to about 500%, from about 25% to about 250%, from about 25% to about 100%, from about 100% to about 5,000%, from about 100% to about 2,500%, from about 100% to about 1000%, from about 100% to about 500%, or from about 100% to about 250%. An increase in RNA-dependent DNA polymerase activity for a polymerase of the invention may also be measured according to relative activity compared to (1) the corresponding unmutated or wild-type enzyme; or (2) a particular polymerase (e.g., Tth polymerase) or group of polymerases. Preferably, the increase in such relative activity is at least about 1.1, 1.2, 1.5, 2, 5, 10, 25, 50, 75, 100, 150, 200, 300, 500, 1,000, 2,500, 5,000, 10,000, or 25,000 fold when the activity of a polymerase of the invention is compared to (1) the corresponding unmutated or wild-type enzyme; or (2) a particular polymerase (e.g., Thermus thermophilus (Tth) polymerase) or group of polymerases. Thus a mutant polymerase of the invention may have an increased RNA-dependent DNA polymerase activity of from about 1.1 fold to about 25,000 fold, from about 1.1 fold to about 10,000 fold, from about 1.1 fold to about 5,000 fold, from about 1.1 fold to about 2,500 fold, from about 1.1 fold to about 1,000 fold, from about 1.1 fold to about 500 fold, from about 1.1 fold to about 250 fold, from about 1.1 fold to about 50 fold, from about 1.1 fold to about 25 fold, from about 1.1 fold to about 10 fold, from about 1.1 fold to about 5 fold, from about 5 fold to about 25,000 fold, from about 5 fold to about 5,000 fold, from about 5 fold to about 1,000 fold, from about 5 fold to about 500 fold, from about 5 fold to about 100 fold, from about 5 fold to about 50 fold, from about 5 fold to about 25 fold, from about 50 fold to about 25,000 fold, from about 50 fold to about 5,000 fold, from about 50 fold to about 1,000 fold, from about 50 fold to about 500 fold, from about 50 fold to about 100 fold, from about 100 fold to about 25,000 fold, from about 1,000 fold to about 25,000 fold, from about 4,000 fold to about 25,000 fold, from about 10,000 fold to about 25,000 fold, from about 15,000 fold to about 25,000 fold, from about 1,000 fold to about 10,000 fold, from about 2,500 fold, to about 10,000 fold, from about 5,000 fold to about 10,000 fold, from about 7,500 fold to about 10,000 fold, from about 1,000 fold to about 15,000 fold, from about 2,500 fold, to about 15,000 fold, from about 5,000 fold to about 15,000 fold, from about 7,500 fold to about 15,000 fold, from about 10,000 fold to about 15,000 fold, or from about 12,500 fold to about 15,000 fold.

Alternatively, the increase in the RNA-dependent DNA polymerase activity of the mutant polypeptides of the invention over that of the un-mutated wildtype polymerase may be measured directly as an increase in specific activity. After mutation, the specific activity of the polypeptides of the invention may be at least about 150, 250, 500, 750, 1,000, 2,000, 3,000, 4,000, 5,000, 7,500, 10,000, 15,000, 25,000, 50,000, 75,000, 100,000, 250,000, or 500,00 units of RNA-dependent DNA polymerase activity/mg protein. Thus, the specific activity of polypeptides of the invention may range from about 150 to about 10,000, from about 150 to about 7,500, from about 150 to about 5,000, from about 150 to about 4,000, from about 150 to about 3,000, from about 150 to about 2,000, from about 150 to about 1,000, from about 150 to about 500, from about 150 to about 250, from about 250 to about 10,000, from about 250 to about 7,500, from about 250 to about 5,000, from about 250 to about 4,000, from about 250 to about 3,000, from about 250 to about 2,000, from about 250 to about 1,000, from about 250 to about 500, from about 500 to about 10,000, from about 500 to about 7,500, from about 500 to about 5,000, from about 500 to about 4,000, from about 500 to about 3,000, from about 500 to about 2,000, or from about 500 to about 1,000 units/mg protein. One unit of RNA-dependent DNA polymerase activity is the amount of enzyme required to incorporate 10 nmoles of dNTPs into acid insoluble product in 30 min using assay conditions described herein (e.g., those in the Examples).

In some embodiments, the polypeptides of the invention incorporate dideoxynucleotides into a DNA molecule about as efficiently as deoxynucleotides. In some embodiments, the polypeptides of the invention may have one or more mutations that substantially change (e.g., reduce or increase) an exonuclease activity, for example, a 5′-3′ exonuclease activity and/or a 3′-5′ exonuclease activity. A polypeptide of the invention, for example, a mutant DNA polymerase of this invention, can exhibit one or more of these properties. Mutant polypeptides of the present invention may also be used in reverse transcription/amplification reactions, DNA sequencing, amplification reactions, and cDNA synthesis.

In some embodiments, the present invention provides polypeptides having an RNA-dependent DNA polymerase activity, i.e., a reverse transcriptase activity. Preferably, the RNA-dependent polymerase activity occurs in the presence of magnesium and/or manganese and/or mixtures of magnesium and manganese. The RNA-dependent polymerase activity may occur in the presence of a mixture of Mn²⁺ and Mg²⁺ preferably at a Mn²⁺:Mg²⁺ ratio of from about 50:1 to 1:50, or from about 10:1 to 1:50, or from about 5:1 to 1:50, or from about 1:1 to 1:50, or from about 50:1 to 1:10, or from about 50:1 to 1:5, or from about 50:1 to 1:1, or from about 10:1 to 1:10, or from about 5:1 to 1:10 or from about 1:1 to 1:10, or from about 10:1 to 1:5, or from 10:1 to 1:1, or from 5:1 to 1:5, or from 5:1 to 1:1, or from 1:1 to 1:5. Concentrations of either divalent cation may range from about 0.1 mM to about 100 mM, from about 0.1 mM to about 50 mM, from about 0.1 mM to about 25 mM, from about 0.1 mM to about 20 mM, from about 0.1 mM to about 15 mM, from about 0.1 mM to about 10 mM, from about 0.1 mM to about 5 mM, from about 0.1 mM to about 1 mM, or from about 0.1 mM to about 0.5 mM. Concentrations of either divalent cation may range from about 0.5 mM to about 100 mM, from about 0.5 mM to about 50 mM, from about 0.5 mM to about 25 mM, from about 0.5 mM to about 20 mM, from about 0.5 mM to about 15 mM, from about 0.5 mM to about 10 mM, from about 0.5 mM to about 5 mM, or from about 0.5 mM to about 1 mM. Concentrations of either divalent cation may range from about 1 mM to about 100 mM, from about 1 mM to about 50 mM, from about 1 mM to about 25 mM, from about 1 mM to about 20 mM, from about 1 mM to about 15 mM, from about 1 mM to about 10 mM, from about 1 mM to about 5 mM, or from about 1 mM to about 2.5 mM.

Polypeptides of the invention may display both an RNA-dependent DNA polymerase activity and a DNA-dependent DNA polymerase activity. When polypeptides of the invention display both activities, the DNA-dependent activity may occur under the same ratio of Mn²⁺:Mg²⁺ as the RNA-dependent polymerase activity. In some embodiments, the DNA-dependent DNA polymerase activity and the RNA-dependent DNA polymerase activity may both occur at ratios of Mn²⁺:Mg²⁺ that overlap. Different portions of the overlap may control the relative amounts of DNA-dependent and RNA-dependent DNA polymerase activity.

In some embodiments, polypeptides of the invention may display an RNA-dependent DNA polymerase activity in the presence of Mg²⁺ and the activity may not require the presence of Mn²⁺.

In some embodiments, the polypeptides of the present invention have reverse transcriptase activity at temperatures above about 50° C. The polypeptides preferably retain activity during or after exposure to elevated temperatures, for example temperatures of about 45° C., 50° C., 55° C., 60° C., 62° C., 65° C., 68° C., 70° C., 72° C. or 75° C. or higher, even up to 80° C., 85° C., 95° C. or 100° C. at ambient or elevated pressure. In additional aspects, the invention also includes polypeptides that retain at least about 50%, at least about 60%, at least about 70%, at least about 85%, at least about 95%, at least about 97%, at least about 98%, at least about 99%, at least about 100%, at least about 150%, at least about 200%, at least about 250%, or at least about 300% of reverse transcriptase activity after heating to about 50° C., about 55° C., about 60° C., about 65° C., about 70° C., about 75° C., about 80° C., about 85° C., about 90° C., or about 95° C. for from about 1 to about 30 minutes, from about 1 to about 25 minutes, from about 1 to about 20 minutes, from about 1 to about 15 minutes, from about 1 to about 10 minutes, from about 1 to about 5 minutes, from about 1 to about 2.5 minutes, from about 2.5 to about 30 minutes, from about 2.5 to about 25 minutes, from about 2.5 to about 20 minutes, from about 2.5 to about 15 minutes, from about 2.5 to about 10 minutes, from about 2.5 to about 5 minutes, from about 5 to about 30 minutes, from about 5 to about 25 minutes, from about 5 to about 20 minutes, from about 5 to about 15 minutes, or from about 5 to about 10 minutes. Preferably, this activity is evident in the presence of magnesium and can be optimized in the presence of other additives. Polypeptides of the invention are useful for procedures requiring reverse transcription. Included within the scope of the present invention are various mutants including deletion, substitution, and insertion mutants that retain or improve thermostability and the ability to replicate DNA preferably with substantially the same efficiency or improved efficiency as that of native thermophilic eubacterial DNA polymerase.

Exemplary purified enzymes of the present invention have a molecular weight of about 100 kilodaltons when measured on SDS-PAGE. They may possess 5′-3′ exonuclease activity and/or 3′-5′ exonuclease activity. In some embodiments, polypeptides of the invention may comprise one or more mutations that reduces, substantially reduces or substantially eliminates one or more exonuclease activity. The present invention also generally includes DNA polymerases that have mutations that reduce, substantially reduce, or eliminate 5′-3′ exonuclease activity. The present invention also generally includes DNA polymerases that have mutations that reduce, substantially reduce, or eliminate 3′-5′ exonuclease activity.

In some embodiments, a polypeptide of the invention may have a temperature optimum that is greater than about 37° C. for one or more enzymatic activities. In some embodiments, polypeptides of the invention may have a temperature optimum for DNA polymerase activity, DNA- and/or RNA-dependent DNA polymerase activity, of at least 50° C., at least 55° C., at least 60° C., at least 65° C., at least 75° C., at least 80° C., or at least 90° C. In some embodiments, polypeptides of the invention may have a temperature optimum for DNA polymerase activity of from about 50° C. to about 90° C., from about 55° C. to about 90° C., from about 60° C. to about 90° C., from about 65° C. to about 90° C., from about 70° C. to about 90° C., from about 75° C. to about 90° C., from about 80° C. to about 90° C., or from about 85° C. to about 90° C. In some aspects, polypeptides of the invention may have a temperature optimum for DNA polymerase activity of from about 50° C. to about 85° C., from about 50° C. to about 80° C., from about 50° C. to about 75° C., from about 50° C. to about 70° C., from about 50° C. to about 65° C., from about 50° C. to about 60° C., or from about 50° C. to about 55° C. Temperature optima may be determined using assay conditions described herein.

Preferably polypeptides of the invention are active in the presence of manganese and/or magnesium. In one embodiment the enzyme is active in the presence of manganese in excess or even great excess over magnesium. Magnesium is not necessarily present for some embodiments of the present invention. In some embodiments, the polypeptides of the invention are active in the presence of magnesium. In one embodiment, the polypeptides of the invention exhibit RT activity in the presence of magnesium.

In one aspect, the present invention provides a composition comprising a polypeptide of the invention (e.g., a wildtype polypeptide, a mutant polypeptide, a fragment of a wildtype polypeptide and/or a fragment of a mutant polypeptide of the invention). In some embodiments, the polypeptide may have a DNA-dependent DNA polymerase activity and/or an RNA-dependent DNA polymerase activity. In some embodiments, one or more of these activities is thermostable. In some embodiments, the polypeptide possesses both activities and both activities are thermostable. The polypeptides may be present as intact polypeptides or may be present as fragments comprising either or both DNA polymerase activities. Compositions may comprise one or more template nucleic acid molecules that may be RNA, DNA, analogues of RNA and/or DNA or a mixture of these. Compositions may comprise one or more nucleoside triphosphates and/or analogs and/or derivatives thereof. Nucleoside triphosphates may be ribonucleosides (rNTPs), deoxyribonucleosides (dNTPs), dideoxynucleosides (ddNTPs) or mixtures thereof. Nucleoside triphosphates may contain one or more detectable groups or moieties, including, but not limited to fluorescent moieties and radioactive moieties. Compositions of the invention may comprise one or more additional polypeptides that may have one or more catalytic activities. An additional polypeptide may or may not have at least one region (e.g., domain) that is substantially homologous to a region of the polypeptide of the invention. In some embodiments, a composition of the invention may comprise a polypeptide of the invention and an additional polypeptide having a DNA polymerase activity. Compositions of this type may further comprise the ingredients listed above, for example, may comprise one or more nucleoside triphosphates, templates and the like. In one embodiment, a composition of the present invention may comprise a polypeptide of the invention, an additional polypeptide having a DNA polymerase activity, a nucleic acid template such as an mRNA, one or more nucleoside triphosphates, and suitable buffers or buffering salts, cofactors and the like to conduct a combined reverse transcription/polymerase chain reaction (RT-PCR). In some embodiments, compositions of the invention may comprise a divalent metal (e.g., Mg²⁺, Mn²⁺, etc.). In some embodiments, compositions may comprise Mg²⁺ and not comprise Mn²⁺.

In another embodiment, the present invention provides a nucleic acid molecule encoding a polypeptide of the present invention or a mutant and/or fragment thereof. Mutants and/or fragments may comprise one or more activities associated with the wild type polypeptide. In some embodiments, the present invention provides nucleic acid molecules encoding mutants, fragments and/or fragments of mutant DNA polymerases. In some embodiments, nucleic acids of the invention may encode all or a portion of a wild type or mutant polymerases from a thermophilic eubacteria including, but not limited to Clostridium spp. (e.g., Clostridium stercorarium, Clostridium thermosulfurogenes, etc.), Caldibacillus spp. (e.g., Caldibacillus cellulovorans CompA.2), Caldicellulosiruptor spp. (e.g., Caldicellulosiruptor Tok13B, Caldicellulosiruptor Tok7B, Caldicellulosiruptor RT69B), Bacillus spp. (e.g., Bacillus caldolyticus EA1), Thermus spp. (e.g., Thermus RT41A), and Dictyoglomus spp. (e.g., Dictyoglomus thermophilum). Specifically, DNA polymerases encoded by the nucleic acid molecules of the present invention may be wild type or may have one or more mutations and/or deletions that increase/decrease one or more desirable/undesirable characteristic of the polypeptide. For example, the present invention provides nucleic acids encoding polypeptides with mutations that result in enhanced thermo stability of the polymerase and/or mutations that result in the ability or improved ability of the mutant DNA polymerase to, under selected conditions, incorporate dideoxynucleotides into a DNA molecule. In some embodiments, the polypeptides encoded by the nucleic acid molecules of the invention incorporate dideoxynucleotides into a DNA molecule about as efficiently as deoxynucleotides. In some embodiments, the polypeptides encoded by the nucleic acid molecules of the invention may have one or more mutations that substantially reduce or increase an exonuclease activity, for example, a 5′-3′ exonuclease activity and/or a 3′-5′ exonuclease activity. A polypeptide encoded by a nucleic acid molecule of the invention, for example, a mutant DNA polymerase of this invention, can exhibit one or more of these properties.

In some embodiments, the present invention is also directed to a nucleic acid encoding a DNA polymerase from a thermophilic eubacterium. Such nucleic acids may comprise all or a portion of one or more of SEQ ID NOS: 2-13. The present invention also comprises a nucleic acid that encodes a polypeptide having all or a portion of one or more of the amino acid sequences of any one of SEQ ID NOS:14-25 representing the translations of the open reading frames of SEQ ID NOs:2-13. The present invention also encompasses polypeptides having at least 80% amino acid identity, preferably at least 90% identity, to at least 15, 20, 25, 30, 35, 40, 45, 50, 60, 70, 80, 90, 100, 150, 200, 250, 275, 300, 350, 400 or 450 contiguous amino acids of the sequence shown in any one of SEQ ID NOS:14-25. Typically, these polypeptides may possess one or more desirable activities, such as, DNA-dependent DNA polymerase activity, RT activity and/or exonuclease activity. The present invention also encompasses nucleic acid molecules encoding such polypeptides.

Nucleic acid molecules of the invention can be introduced into host cells and host cells expressing the polypeptides encoded by the nucleic acid molecules of the invention may be prepared. Any type or strain of host cell may be used to express the polypeptides of the present invention including prokaryotic and eukaryotic cells. In vitro cell free expression systems can also be used to express the polymerases of the present invention. Preferably, prokaryotic cells are used to express the polypeptides of the invention. A preferred prokaryotic host according to the present invention is E. coli.

The present invention also provides reaction conditions in which DNA polymerases, for example, some polymerases known in the prior art, exhibit a polymerase activity, for example, an RT activity. Such conditions preferably comprise a lower monovalent cation concentration than was previously employed. In some embodiments, the monovalent cation concentration is from about 1 mM to about 100 mM, from about 1 mM to about 75 mm, from about 1 mM to about 50 mM, from about 1 mM to about 40 mM, from about 1 mM to about 30 mM, from about 1 mM to about 25 mM, from about 1 mM to abut 20 mM, from about 1 mM to about 15 mM, from about 1 mM to about 10 mM, from about 1 mM to about 5 mM, from about 1 mM to about 2.5 mM, from about 5 mM to about 100 mM, from about 5 mM to about 75 mm, from about 5 mM to about 50 mM, from about 5 mM to about 40 mM, from about 5 mM to about 30 mM, from about 5 mM to about 25 mM, from about 5 mM to abut 20 mM, from about 5 mM to about 15 mM, from about 5 mM to about 10 mM, from about 10 mM to about 100 mM, from about 10 mM to about 75 mm, from about 10 mM to about 50 mM, from about 10 mM to about 40 mM, from about 10 mM to about 30 mM, from about 10 mM to about 25 mM, from about 10 mM to abut 20 mM, or from about 10 mM to about 15 mM. In some embodiments, the monovalent cation concentration is about 25 mM. Monovalent cations include, but are not limited to, lithium, potassium, sodium and ammonium. Suitable sources of monovalent cations include, but are not limited to, LiCl, KCl, NaCl, and (NH₄)₂SO₄. In some embodiments, the present invention provides conditions under which a polymerase enzyme exhibits an RT activity in the absence of Mn²⁺. The present invention also provides compositions comprising a thermostable DNA polymerase and monovalent cation, wherein the total concentration of monovalent cations is from about 0.1 mM to about 60 mM, from about 1 mM to about 60 mM from about 2 mM about 60 mM, from about 5 mM to about 60 mM, from about 5 mM to about 50 mM, from about 5 mM to about 40 mM, from about 5 mM to about 30 mM, from about 5 mM to about 20 mM or from about 5 mM to about 10 mM. Such compositions may further comprise one or more template molecules, which may by DNA or RNA and are preferably mRNA, one or more nucleotides, one or more divalent metals (e.g., Mg²⁺), one or more primers, and/or one or more buffers or buffer salts.

The present invention also relates to polypeptides of the invention that have multiple mutations such that the polypeptides lack or substantially lack exonuclease activity (5′-3′ and/or 3′-5′) and are nondiscriminatory against ddNTPs in sequencing reactions. These mutants may exhibit exonuclease activity under some specific conditions, but may lack or substantially lack the exonuclease activity under conditions used in reverse transcription and/or polymerization.

Preferred polypeptides of the invention relate to mutant polypeptides that are modified in at least one way selected from the group consisting of (a) to reduce or eliminate the 5′-3′ exonuclease activity of the polymerase; (b) to reduce or eliminate the 3′-5′ exonuclease activity of the polypeptide; (c) to reduce or eliminate discriminatory behavior against one or more dideoxynucleotides; (d) to enhance thermostability of one or more enzymatic activities of the polypeptide; (e) to enhance reverse transcriptase activity of the polypeptide (e.g., in the presence of Mg²⁺); and (f) combinations of two or more of (a) to (e). Each activity may be modified alone or in conjunction with a modification of another activity (e.g., 3′-5′ exonuclease activity can be modified or eliminated independently of actions affecting 5′-3′ exonuclease activity).

The present invention also relates to antibodies that specifically bind to the polypeptides of the invention. Such antibodies include fragments of antibodies that retain the ability to bind to the polypeptides of the invention. Such antibodies may bind to polypeptides of the invention at one temperature (e.g., a lower temperature) and may not bind to polypeptides of the invention at a second temperature (e.g., a higher temperature). Such antibodies may be useful in the practice of one or more methods of the invention to permit the use of a “hot start.” A hot start is one in which one or more activities of the polypeptides of the invention is inhibited at a temperature below a desired starting temperature and is not inhibited or is less inhibited at or above the desired temperature.

The invention also relates to a method of producing a DNA polymerase, the method comprising:

(a) culturing a host cell of the invention;

(b) expressing a DNA polymerase in the host cell; and

(c) isolating the DNA polymerase from the host cell.

The invention also relates to a method of synthesizing a nucleic acid molecule, the method comprising:

(a) mixing one or more template nucleic acid molecules with one or more polypeptides of the invention to form a mixture; and

(b) incubating the mixture under conditions sufficient to synthesize a nucleic acid molecule complementary to all or a portion of the template. In accordance with the invention, the synthesized nucleic acid molecule may be used as a template under appropriate conditions to synthesize nucleic acid molecules complementary to all or a portion of the templates, thereby forming double stranded nucleic acid molecules. In yet another aspect, the synthesized double stranded molecules may be amplified. In some embodiments, conditions sufficient to synthesize one or more nucleic acid molecules according to the invention may include one or more nucleotides, one or more buffers or buffering salts, one or more primers, one or more cofactors (e.g., divalent metal ions), and/or one or more additional polypeptides having a nucleotide polymerase activity. In some embodiments, conditions sufficient to synthesize one or more nucleic acid molecules according to the invention may include incubating at an elevated temperature (e.g., greater than about 37° C., 40° C., 45° C., 50° C., 55° C., 60° C., 65° C., 70° C., 75° C., 80° C., 85° C., 90° C., or 95° C.) and/or in the presence of one or more deoxy- or dideoxyribonucleoside triphosphates. Suitable deoxy- and dideoxyribonucleoside triphosphates include, but are not limited to, dATP, dCTP, dGTP, dTTP, dITP, 7-deaza-dGTP, 7-deaza-dATP, ddUTP, ddATP, ddCTP, ddGTP, ddITP, ddTTP, [α-S]dATP, [α-S]dTTP, [α-S]dGTP, and [α-S]dCTP. In some embodiments, the conditions may comprise a suitable concentration of at least one divalent metal cofactor. In some embodiments, the conditions may comprise more than one divalent metal cofactor. In some embodiments, the conditions may comprise Mg²⁺ and not Mn²⁺.

The invention also relates to a method of synthesizing a nucleic acid molecule, the method comprising:

(a) mixing one or more template nucleic acid molecules with one or more polypeptides of the invention to form a mixture, wherein the polypeptide is in a complex with a molecule that inhibits one or more activity of the polypeptide; and

(b) incubating the mixture under conditions sufficient to synthesize a nucleic acid molecule complementary to all or a portion of the template. In some embodiments, the polypeptide may be in a complex with an antibody that inhibits one or more activity of the polypeptide at a first temperature (e.g., inhibits a DNA-dependent and/or an RNA-dependent polymerase activity) and does not inhibit or inhibits to a lessor extent the activity at a second temperature. Such methods may further comprise performing step (a) at a first temperature and performing step (b) at a second temperature wherein the temperature of step (b) is greater than the temperature of step (a). In some embodiments, the second temperature may be greater than about 40° C., 45° C., 50° C., 55° C., 60° C., 65° C., 70° C., 75° C., 80° C., 85° C., 90° C., or 95° C. Methods of this type may be used to produce a nucleic acid molecule (e.g., a cDNA molecule) complementary to all or a portion of one or more mRNA template molecules and/or populations of mRNA template molecules. In accordance with the invention, the synthesized nucleic acid molecule may be used as a template under appropriate conditions to synthesize nucleic acid molecules complementary to all or a portion of the templates, thereby forming double stranded nucleic acid molecules. In yet another aspect, the synthesized double stranded molecules may be amplified. In some embodiments, conditions sufficient to synthesize one or more nucleic acid molecules according to the invention may include one or more nucleotides, one or more buffers or buffering salts, one or more primers, one or more cofactors (e.g., divalent metal ions), and/or one or more additional polypeptides having a nucleotide polymerase activity. In some embodiments, conditions sufficient to synthesize one or more nucleic acid molecules according to the invention may include incubating at an elevated temperature (e.g., greater than about 37° C., 40° C., 45° C., 50° C., 55° C., 60° C., 65° C., 70° C., 75° C., 80° C., 85° C., 90° C., or 95° C.) and/or in the presence of one or more deoxy- or dideoxyribonucleoside triphosphates. Suitable deoxy- and dideoxyribonucleoside triphosphates include, but are not limited to, dATP, dCTP, dGTP, dTTP, dITP, 7-deaza-dGTP, 7-deaza-dATP, ddUTP, ddATP, ddCTP, ddGTP, ddITP, ddTTP, [α-S]dATP, [α-S]dTTP, [α-S]dGTP, and [α-S]dCTP. In some embodiments, the conditions may comprise a suitable concentration of at least one divalent metal cofactor. In some embodiments, the conditions may comprise more than one divalent metal cofactor. In some embodiments, the conditions may comprise Mg²⁺ and not Mn²⁺.

In some embodiments, the present invention provides a method of making cDNA molecules. In accordance with the invention, cDNA molecules (single-stranded or double-stranded) may be prepared from a variety of nucleic acid template molecules. Preferred nucleic acid molecules for use in the present invention include single-stranded RNA molecules, as well as double-stranded DNA:RNA hybrids. More preferred nucleic acid molecules include messenger RNA (mRNA), transfer RNA (tRNA) and ribosomal RNA (rRNA) molecules, although mRNA molecules are the preferred template according to the invention. Such methods may comprise:

(a) mixing one or more RNA templates (e.g., mRNA) or a population of RNA templates with a polypeptide of the invention to form a mixture; and

(b) incubating said mixture under conditions sufficient to synthesize one or more nucleic acid molecules which are complementary to all or a portion of said templates. In accordance with the invention, the synthesized nucleic acid molecule may be used as a template under appropriate conditions to synthesize nucleic acid molecules complementary to all or a portion of the templates, thereby forming double stranded molecules. In yet another aspect, the synthesized double stranded molecules may be amplified. In some embodiments, conditions sufficient to synthesize one or more nucleic acid molecules according to the invention may include incubating at an elevated temperature (e.g., greater than about 37° C., 40° C., 45° C., 50° C., 55° C., 60° C., 65° C., 70° C., 75° C., 80° C., 85° C., 90° C., or 95° C.)) and/or in the presence of one or more deoxy- or dideoxyribonucleoside triphosphates, one or more of which may comprise a label (e.g., a fluorescent label, a radioactive label, a detectable moiety, a reactive moiety, etc.). Suitable deoxy- and dideoxyribonucleoside triphosphates include, but are not limited to, dATP, dCTP, dGTP, dTTP, dITP, 7-deaza-dGTP, 7-deaza-dATP, ddUTP, ddATP, ddCTP, ddGTP, ddITP, ddTTP, [α-S]dATP, [α-S]dTTP, [α-S]dGTP, and [α-S]dCTP. In some embodiments, the conditions may comprise a suitable concentration of at least one divalent metal cofactor. In some embodiments, the conditions may comprise more than one divalent metal cofactor. In some embodiments, the conditions may comprise Mg2+ and not Mn2+. The method may optionally comprise

(c) treating the reaction mixture to provide single stranded cDNA;

(d) hybridizing a second primer to the cDNA molecule in the presence of the polypeptide of the invention, under conditions such that an extension product is synthesized to provide a double-stranded cDNA molecule; and

(e) amplifying the double-stranded cDNA molecule of (d) (e.g., by a polymerase chain reaction). In one embodiment, amplification using the polymerase chain reaction is by a polymerase other than that of the present invention. Any thermostable polymerase used in polymerase chain reactions can be used, for example Taq DNA polymerase. The use of the polypeptides of the present invention allows the use of other DNA polymerases in the same buffer solution. In some embodiments, methods of the invention may further comprise isolating one or more cDNA molecules produced by the methods of the invention.

In another aspect of the invention, the present invention provides methods of amplifying one or more nucleic acid molecules. Such methods may comprise:

(a) mixing one or more templates with one or more primers and one or more polypeptides of the invention; and

(b) incubating said mixture under conditions sufficient to amplify said one or more templates. In particular, one or more template molecules may be double stranded nucleic acid molecules and such amplification methods may comprise:

(a) contacting a first strand of the nucleic acid template molecule with a first primer molecule which is complementary to a portion of said first strand and a second strand of the nucleic acid template molecule with a second primer molecule which is complementary to a portion of said second strand in the presence of one or more polypeptides of the invention;

(b) incubating said molecules under conditions sufficient to form a third strand complementary to all or a portion of said first strand and a fourth strand complementary to all or a portion of said second strand;

(c) denaturing said first and third and said second and fourth strands; and

(d) repeating steps (a) through (c) one or more times. In some embodiments, such conditions according to the invention may include one or more nucleotides, one or more buffers or buffering salts, one or more primers, one or more cofactors, and/or one or more additional polypeptides having a nucleotide polymerase activity (which may be polypeptides of the invention or otherwise).

The invention also relates to a method of sequencing a nucleic acid molecule, comprising:

(a) hybridizing a primer to a first nucleic acid molecule to form a complex comprising the nucleic acid molecule and the primer;

(b) contacting the complex of (a) with one or more deoxyribonucleoside triphosphates, a polypeptide of the invention, and at least one terminator nucleotide to form a mixture;

(c) incubating the mixture of (b) under conditions sufficient to synthesize a population of DNA molecules complementary to the first nucleic acid wherein a detectable portion of the synthesized DNA molecules comprise a terminator nucleotide at their respective 3′ termini; and

(d) separating the population of synthesized DNA molecules by size or assaying the population so that at least a part of the nucleotide sequence of the first nucleic acid molecule can be determined. Exemplary terminator nucleotides include ddTTP, ddATP, ddGTP, ddITP or ddCTP each of which may comprise a detectable moiety. In some embodiments, each will comprise a detectable moiety and each moiety will be different.

The invention also relates to a method for amplifying all or a portion of a double stranded DNA molecule, comprising:

(d) providing a first and second primer, wherein the first primer is complementary to a sequence at or near the 5′-terminus of a portion desired to be amplified of a first strand of the DNA molecule and the second primer is complementary to a sequence at or near the 3′-terminus of a portion desired to be amplified of a second strand of the DNA molecule;

(e) hybridizing the first primer to the first strand and the second primer to the second strand in the presence of a polypeptide of the invention, under conditions such that a third DNA molecule complementary to at least a portion of the first strand and a fourth DNA molecule complementary to at least a portion of the second strand are synthesized;

(c) denaturing the first and third strand, and the second and fourth strands; and optionally

(d) repeating steps (a) to (c) one or more times.

The invention also relates to a kit for sequencing a nucleic acid molecule, comprising one or more containers containing one or more of the following:

(a) a polypeptide of the invention;

(b) one or more dideoxyribonucleoside triphosphates, one or more of which may comprise a label (e.g., a fluorescent label, a radioactive label, a detectable moiety, a reactive moiety, etc.).; and

(c) one or more deoxyribonucleoside triphosphates.

The invention also relates to a kit for RT/PCR, comprising one or more containers containing one or more of the following:

(a) a polypeptide of the invention;

(b) one or more deoxyribonucleoside triphosphates, one or more of which may comprise a label (e.g., a fluorescent label, a radioactive label, a detectable moiety, a reactive moiety, etc.); and

(c) a thermostable DNA polymerase.

### BRIEF DESCRIPTION OF THE DRAWINGS/FIGURES

FIG. 1. An alignment of known bacterial DNA polI gene sequences at the position of two highly conserved amino acid motifs. Degenerate oligonucleotides designed to amplify the equivalent region from other bacterial polymerases are shown beneath the alignment.

FIG. 2. SDS-PAGE analysis of the purified DNA polymerases. Approximately 1 μg of each purified DNA polymerase was subjected to electrophoresis on a 4-20% Tris-glycine gel and stained using Gel-code Blue (Materials and Methods). Benchmark Protein Ladder was run as a standard on the left and the right sides of the samples and the molecular weight (kDa) of each band is labeled on the left side of the figure.

FIG. 3. Alkaline-agarose gel analysis of first-strand cDNA synthesized from CAT cRNA by purified thermostable DNA polymerases. CAT cRNA was reverse transcribed using a 24 bp gene specific DNA primer in the presence (+) and absence (−) of betaine. The cDNA products were subjected to electrophoresis on an alkaline 2% agarose gel. A 100 bp DNA ladder was used as a standard.

FIG. 4 is a bar graph showing the effects of KCl concentration on Mg2+-dependent reverse transcriptase activity for Clostridium stercorarium (C. sterco), Caldibacillus cellulovorans CompA.2 (CompA2) and Clostridium thermosulfurogenes (C. thermo) DNA polymerases. SUPERSCRIPT™ II (SSII, a modified M-MLV reverse transcriptase) was included as a control.

FIG. 5 is a bar graph shows the results of a comparison of the reverse transcriptase activity of varying amounts of the polymerases of the invention in the presence and absence of Betaine.

FIG. 6 is an autoradiograph of reverse transcriptase activity of several polymerases of the invention in the presence and absence of Betaine in low salt buffer.

FIG. 7 is an autoradiograph showing reverse transcriptase activity of several polymerases of the invention in the presence and absence of Betaine.

### DETAILED DESCRIPTION OF THE INVENTION

### Definitions

In the description that follows, a number of terms used in recombinant DNA technology are extensively utilized. In order to provide a clearer and consistent understanding of the specification and claims, including the scope to be given such terms, the following definitions are provided.

Cloning vector. A nucleic acid molecule, for example, a plasmid, cosmid or phage DNA or other DNA molecule, that is able to replicate autonomously in a host cell. A cloning vector may have one or a small number of recognition sites (e.g., recombination sites, restriction sites, topoisomerase sites, etc.) at which such DNA sequences may manipulated in a determinable fashion without loss of an essential biological function of the vector, and into which a nucleic acid segment of interest may be inserted in order to bring about its replication and cloning. The cloning vector may further contain a marker suitable for use in the identification of cells transformed with the cloning vector. Markers may be, for example, antibiotic resistance genes such as tetracycline resistance, ampicillin resistance or kanamycin resistance genes. Any other marker sequence known to those skilled in the art may be used.

Expression vector. A vector similar to a cloning vector but which is capable of enhancing the expression of a gene that has been cloned into it, after transformation into a host. The cloned gene is usually placed under the control of (i.e., operably linked to) certain control sequences such as promoter or enhancer sequences.

Recombinant host. Any prokaryotic cell or eukaryotic cell or microorganism which contains the desired cloned gene in an expression vector, cloning vector or any heterologous nucleic acid molecule. The term “recombinant host” is also meant to include those host cells which have been genetically engineered to contain the desired genes as part of the host chromosome or genome.

Host. Any prokaryotic cell or eukaryotic cell or microorganism that is the recipient of a replicable expression vector, cloning vector or any heterologous nucleic acid molecule. The nucleic acid molecule may contain, but is not limited to, a structural gene, or portion thereof, a promoter and/or an origin of replication.

Promoter. A DNA sequence to which an RNA polymerase binds such that the polymerase, in the presence of the appropriate cofactors, initiates transcription at a transcriptional start site of a nucleic acid sequence to be transcribed. RNA polymerase catalyzes the synthesis of messenger RNA complementary to the appropriate DNA strand of the coding region. Promoter also includes any 5′ non-coding region that may be present between the transcriptional start site and the translation start site. Promoter also includes cis-acting transcription control elements such as enhancers, and other nucleotide sequences capable of interacting with transcription factors.

Operably linked. As used herein means that the promoter or other control sequence, such as an enhancer, is positioned to control the transcription from a sequence operably linked thereto.

Expression. Expression is the process by which a polypeptide is produced from a nucleic acid. It may include transcription of a gene into messenger RNA (mRNA) and the translation of such mRNA into polypeptide(s).

Substantially Pure. As used herein “substantially pure” means that the desired purified protein is essentially free from contaminating cellular contaminants which are associated with the desired protein in nature and that unacceptably impair the desired function. Contaminating cellular components may include, but are not limited to, one or more phosphatases, exonucleases, endonucleases or undesirable DNA polymerase enzymes. In a preferred aspect, a polypeptide of the invention has 25% or less, preferably 15% or less, more preferably 10% or less, more preferably 5% or less, and still more preferably 1% or less contaminating cellular components. In another aspect, the polypeptides of the invention have no detectable protein contaminants when 200 units (DNA-dependent DNA polymerase units or RNA-dependent DNA polymerase units) of polypeptide are run on a protein gel (e.g., SDS-PAGE) and stained with Comassie blue. Preferably, polypeptides of the invention are substantially pure.

Substantially isolated. As used herein “substantially isolated” means that the polypeptide of the invention is essentially free from contaminating proteins, which may be associated with the polypeptide of the invention in nature and/or in a recombinant host. In one aspect, a substantially isolated polypeptide of the invention has 25% or less, preferably 15% or less, more preferably 10% or less, more preferably 5% or less, and still more preferably 1% or less contaminating proteins. In another aspect, in a sample of a substantially isolated polypeptide of the invention, 75% or greater (preferably 80%, 85%, 90%, 95%, 98%, or 99% or greater) of the protein in the sample is the desired polypeptide of the invention. The percentage of contaminating protein and/or protein of interest in a sample may be determined using techniques known in the art, for example, by using a protein gel (e.g., SDS-PAGE) and staining the gel with a protein dye (e.g., Coomassie blue, silver stain, amido black, etc.). In another aspect, the polypeptide of the invention have no detectable protein contaminants when 0.5 .mu.g of polypeptide are run on a protein gel (e.g., SDS-PAGE) and stained with Comassie blue or amido black.

Substantially reduced. An enzyme “substantially reduced” in an enzymatic activity means that the enzyme has less than about 30%, less than about 25%, less than about 20%, more preferably less than about 15%, less than about 10%, less than about 7.5%, or less than about 5%, and most preferably less than about 5% or less than about 2%, or less than about 1% of the activity of the corresponding un-mutated or wildtype enzyme.

Primer. As used herein “primer” refers to a single-stranded oligonucleotide that is extended by covalent bonding of nucleotide monomers during polymerization or amplification of a nucleic acid molecule.

Template. The term “template” as used herein refers to a double-stranded or single-stranded DNA or RNA molecule to be amplified, synthesized, sequenced or copied. In the case of a double-stranded DNA molecule, denaturation of its strands to form a first and a second strand is generally performed before these molecules are amplified, synthesized or sequenced. A primer complementary to a portion of the template is hybridized to the template under appropriate conditions and a polypeptide of the invention may then synthesize a DNA molecule complementary to the template or a portion thereof. Mismatch incorporation during the synthesis or extension of the newly synthesized DNA molecule may result in one or a number of mismatched base pairs. Thus, the synthesized DNA molecule need not be exactly complementary to the template. In the case of RNA, a DNA primer is hybridized to a strand of the template RNA and a polypeptide of the invention having reverse transcriptase activity may be used to synthesize a complementary DNA.

Incorporating. The term “incorporating” as used herein means becoming a part of a nucleic acid molecule or primer.

Amplification. As used herein “amplification” refers to any in vitro method for increasing the number of copies of a nucleotide sequence with the use of a DNA polymerase. Nucleic acid amplification results in the incorporation of nucleotides into a DNA molecule or primer thereby forming a new DNA molecule complementary to a template. The formed DNA molecule and its template can be used as templates to synthesize additional nucleic acid molecules. As used herein, one amplification reaction may consist of many rounds of DNA replication. DNA amplification reactions include, for example, polymerase chain reactions (PCR). One PCR reaction may consist of one or more e.g., 2, 3, 4, 5, 10, 15, 20, 25, 30, 50, 60, 70, 80, 90, 100 or more “cycles” of denaturation and synthesis of a DNA molecule.

Oligonucleotide. “Oligonucleotide” refers to a synthetic or natural molecule comprising a covalently linked sequence of nucleotides or nucleotide analogs. Such nucleotides or nucleotide analogs may be joined by a phosphodiester bond between the 3′ position of the pentose of one nucleotide and the 5′ position of the pentose of the adjacent nucleotide. Also encompassed are molecules in which one or more inter-nucleotide phosphate groups has been replaced by a different type of group, such as, a peptide bond, a phosphorothioate group or a methylene group. Sources of oligonucleotides are not limited. For example, animals, plants, bacteria, viruses, cultured cells, or other organisms may be a source of oligonucleotides. Oligonucleotides may be synthetically prepared. Any class, order, genus, species, or subspecies may be a source, for example, dicot, arthropod, insect, mammal, bovine, ovine, canine, human, murine, rodent, yeast, bacteria, E. coli, etc. can be a source of oligonucleotides.

Nucleotide. As used herein “nucleotide” refers to a base-sugar-phosphate combination. Nucleotides are monomeric units of a nucleic acid sequence (DNA and RNA). The term nucleotide includes deoxyribonucleoside triphosphates such as dATP, dCTP, dITP, dUTP, dGTP, dTTP, or derivatives thereof. Such derivatives include, for example, [α-S]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. According to the present invention, a “nucleotide” may be unlabeled or detectably labeled by well known techniques. Detectable labels include, for example, radioactive isotopes, fluorescent labels, chemiluminescent labels, bioluminescent labels and enzyme labels. Nucleotides for use in the present invention may also comprise one or more reactive functional groups. Labels may be attached to the functional group before, during and/or after use of the nucleotide in a reaction involving a polypeptide of the invention.

According to the present invention, a “nucleotide” may be unlabeled or detectably labeled by well known techniques. Detectable labels include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluroescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink FluorX-dCTP, FluoroLink Cy3-dUTP, and FluoroLink Cy5-dUTP available from Amersham Arlington Heights, Ill.; Fluorescein-15-dATP, Fluorescein-12-dUTP, Tetramethyl-rodamine-6-dUTP, IR₇₇₀-9-dATP, Fluorescein-12-ddUTP, Fluorescein-12-UTP, and Fluorescein-15-2′-dATP available from Boehringer Mannheim Indianapolis, Ind.; and ChromaTide Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg.

Thermostable. As used herein “thermostable” refers to an activity of a molecule that is resistant to inactivation by heat. For example, DNA polymerases synthesize the formation of a DNA molecule complementary to a single-stranded DNA template by extending a primer in the 5′-to-3′ direction. This activity for mesophilic DNA polymerases may be inactivated by heat treatment. For example, T5 DNA polymerase activity is totally inactivated by exposing the enzyme to a temperature of 90° C. for 30 seconds. As used herein, a thermostable activity is more resistant to heat inactivation than a corresponding mesophilic activity. That is, a thermostable DNA polymerase does not refer to an enzyme that is totally resistant to heat inactivation. Thus heat treatment may reduce DNA polymerase activity to some extent in a thermostable polymerase. A thermostable DNA polymerase typically will also have a higher optimum temperature than common mesophilic DNA polymerases. The phrase “thermostable polymerase” is used herein to refer to an enzyme that is relatively stable to heat and is capable of catalyzing the formation of DNA or RNA from an existing nucleic acid template.

A polymerase is considered especially thermostable when it retains at least 5%, or at least 10%, or at least 15%, or at least 20%, or at least 25%, or at least 30%, or at least 35%, or at least 40%, or at least 40%, or at least 45%, or at least 50%, or at least 55%, or at least 60%, or at least 65%, or at least 70%, or at least 75%, or at least 80%, or at least 85%, or at least 90%, or at least 95% of the original polymerase activity after heating, for example, at 95° C. for 30 minutes.

Fidelity. Fidelity refers to the accuracy of polymerization, or the ability of the polymerase to discriminate correct from incorrect substrates, (e.g., nucleotides) when synthesizing nucleic acid molecules (e.g. RNA or DNA) which are complementary to a template. The higher the fidelity of a polymerase, the less the polymerase misincorporates nucleotides in the growing strand during nucleic acid synthesis; that is, an increase or enhancement in fidelity results in a more faithful polymerase having decreased error rate (decreased misincorporation rate).

Hybridization. The terms “hybridization” and “hybridizing” refer to pairing of two complementary single-stranded portions of nucleic acid molecules (RNA and/or DNA) to give a double-stranded molecular portion. As used herein, two nucleic acid molecule portions may be hybridized, although the base pairing is not completely complementary. Accordingly, mismatched bases do not prevent hybridization of two nucleic acid molecule portions provided that appropriate hybridization and stringency conditions, well known in the art, are used.

The ability of two nucleotide sequences to hybridize to each other is based upon a degree of complementarity of the two nucleotide sequences, which in turn is based on the fraction of matched complementary nucleotide pairs. The more nucleotides in a given sequence that are complementary to another sequence, the greater the degree of hybridization of one to the other. The degree of hybridization also depends on the conditions of stringency which include temperature, solvent ratios, salt concentrations, and the like. In particular, “selective hybridization” pertains to conditions in which the degree of hybridization of a polynucleotide of the invention to its target would require complete or nearly complete complementarity. The complementarity must be sufficiently high so as to assure that the polynucleotide of the invention will bind specifically to the target relative to binding other nucleic acids present in the hybridization medium. With selective hybridization, complementarity will be 90-100%, preferably 95-100%, more preferably 100%.

Stringent conditions. The phrase “stringent conditions” refers to conditions under which a nucleic acid probe will hybridize to its target sequence but will not hybridize or will only hybridize to an insubstantial extent with a non-target sequence. Stringent conditions depend upon the length and sequence composition of the probe and target. Longer sequences and sequences with a higher G:C base content hybridize specifically at higher temperatures.

Generally, for a selected ionic strength of hybridization and wash buffer, stringent conditions include a temperature of about 5° C. below the calculated Tₘ for the specific probe and target sequences. Suitable hybridization and wash solutions are known to those skilled in the art and stringent conditions for a given probe and target pair can be determined without undue experimentation by adjusting the salt concentration and temperature until a single or small number of signals is obtained, for example, in a Southern blot. Stringent conditions are typically those that (1) employ low ionic strength and high temperature for washing, for example, 0.015 M NaCl/0.0015 M sodium citrate/0.1% NaDodSO₄ at 50° C., or (2) employ during hybridization a denaturing agent such as formamide, for example, 50% (vol/vol) formamide with 0.1% bovine serum albumin (“BSA”)/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl and 0.075 M sodium citrate), 50 mM sodium phosphate (pH 6.8), 0.1% sodium pyrophosphate, 5×Denhardt's solution, sonicated salmon sperm DNA (50 mg/ml), 0.1% sodium dodecyl sulfate (“SDS”), and 10% dextran sulfate at 42° C., with washes at 42° C. in 0.2×SSC and 0.1% SDS. Other suitable conditions include hybridization at 42° C. in a solution comprising 50% formamide, a first wash at 65° C. in 2×SSC and 1% SDS, and a second wash at 65° C. in 0.1×SSC; and hybridization in 6×SSC 1% SDS, a first was in 6×SSC 1% SDS and a final wash in a solution having a salt concentration of from about 0.05×SSC to about 0.3×SSC and about 0.05% SDS to about 1% SDS at a temperature of from about 50° C. to about 95° C.

3′-to-5′ Exonuclease Activity. “3′-to-5′exonuclease activity” is an enzymatic activity well known to the art in which the 3′-most nucleotide is removed from a polynucleotide. This activity is often associated with DNA polymerases, and is thought to be involved in a DNA replication “editing” or correction mechanism.

Most DNA polymerases contain a 3′-5′ exonuclease activity in addition to polymerase activity. A T5 DNA polymerase that lacks 3′-5′ exonuclease activity is disclosed in U.S. Pat. No. 5,270,179. Polymerases lacking this activity are particularly useful for, e.g., TA Cloning®.

A “DNA polymerase substantially reduced in 3′-to-5′exonuclease activity” is defined herein as either (1) a mutated DNA polymerase that has about or less than 10%, or preferably about or less than 1%, of the 3′-to-5′ exonuclease activity of the corresponding unmutated, wild type enzyme, or (2) a DNA polymerase having a 3′-to-5′ exonuclease specific activity which is less than about 1 unit/mg protein, or preferably about or less than 0.1 units/mg protein. A unit of activity of 3′-to-5′ exonuclease is defined as the amount of activity that solubilizes 10 nmoles of substrate ends in 60 min at 37° C., assayed as described in the “BRL 1989 Catalogue & Reference Guide,” page 5, with HhaI fragments of lambda DNA 3′-end labeled with [³H]dTTP by terminal deoxynucleotidyl transferase (TdT). Protein is measured by the method of Bradford, Anal. Biochem. 72:248 (1976). As a means of comparison, natural, wild type T5-DNA polymerase (DNAP) or T5-DNAP encoded by pTTQ19-T5-2 has a specific activity of about 10 units/mg protein while the DNA polymerase encoded by pTTQ19-T5-2(Exo) (U.S. Pat. No. 5,270,179) has a specific activity of about 0.0001 units/mg protein, or 0.001% of the specific activity of the unmodified enzyme, a 10⁵-fold reduction.

5′-to-3′ Exonuclease Activity. “5′-to-3′ exonuclease activity” is another enzymatic activity well known in the art. This activity is often associated with DNA polymerases, such as E. coli PolI and PolIII. In many of the known polymerases, the 5′-to-3′ exonuclease activity is present in the N-terminal region of the polymerase. (Ollis, et al., Nature 313:762-766 (1985); Freemont, et al., Proteins 1:66-73 (1986); Joyce, Cur. Opin. Struct. Biol. 1:123-129 (1991)). There are some amino acids, the mutations of which are thought to impair the 5′-3′ exonuclease activity of E. coli DNA polymerase I. (Gutman & Minton, Nucl. Acids Res. 21:4406-4407 (1993)). These amino acids include Tyr77, Gly103, Gly184, and Gly192 in E. coli DNA polymerase I. It is known that the 5′-exonuclease domain is dispensable for polymerase activity. The best known example is the Klenow fragment of E. coli polymerase I. The Klenow fragment is a natural proteolytic fragment devoid of 5′-exonuclease activity (Joyce, et al., J. Biol. Chem. 257:1958-64 (1990)). Polymerases lacking this activity are useful for DNA sequencing.

A “DNA polymerase substantially reduced in 5′-to-3′ exonuclease activity” is defined herein as either (1) a mutated DNA polymerase that has about or less than 10%, or preferably about or less than 1%, of the 5′-to-3′ exonuclease activity of the corresponding unmutated, wild type enzyme, or (2) a DNA polymerase having 5′-to-3′ exonuclease specific activity which is less than about 1 unit/mg protein, or preferably about or less than 0.1 units/mg protein.

Both 3′-to-5′ and 5′-to-3′ exonuclease activities can be observed on sequencing gels. Active 5′-to-3′ exonuclease activity will produce nonspecific ladders in a sequencing gel by removing nucleotides from the 5′-end of the growing primers. 3′-to-5′ exonuclease activity can be measured by following the degradation of radiolabeled primers in a sequencing gel. Thus, the relative amounts of these activities, e.g. by comparing wild type and mutant polymerases, can be determined with no more than routine experimentation.

Reverse transcription activity or reverse transcriptase activity. Ability of an enzyme to synthesize a complementary DNA strand from single-stranded portion of RNA. Preferably the activity is sufficient to synthesize a complementary strand at least 10 to 20 nucleotides in length; more preferably the activity is sufficient to synthesize a complementary strand to at least about 20-50, 40-75, 50-100, 75-150, 100-200, 150-300, 200-400, 300-500, 400-600, 500-700, 600-750, 700-1000, 750-1200, 1000-1500, 1200-1800, 1500-2500, 2000-3000, 2500-4000, 3000-5000, 4000-7000, 5000-10000, 7000-15000 or even longer. Of course, an activity sufficient to synthesize a strand at least about 7000-15000 would necessarily be sufficient to synthesize a strand of less than 7000. Preferably the synthesis time is less than one day, preferably less than 4 hours, more preferably less than 60 minutes, 30 minutes, 10 minutes, 5 minutes, 1 minute or ½ minute. Synthesis temperatures are preferably from about 45° C. to about 100° C., including any desired temperature in between, e.g., about 48° C., 50° C., 52° C., 55, 58° C., 60° C., 62° C., 65° C., 68° C., 70° C., 72° C., 75° C., 78° C., 80° C., 82° C., 85° C., 88° C., 90° C., 92° C., 95° C., 98° C. or temperatures in between. Desired temperatures can be selected according to the user's criteria. For example, a desired temperature might be selected as a temperature about the optimum for an enzymatic activity or might be selected for improved availability or stability of the template molecule or synthesized molecule. Stability or inactivation of other substances in the reaction mix might also determine a desired temperature. Activity can be measured under any of these conditions. Presence or absence of activity can be defined functionally. For example, if a synthesis is performed at a desired temperature activity can be defined as the detectable synthesis of a molecule of a desired length. Alternatively a molar, absorbance, weight or other means of measuring may be used to set a threshold for activity.

Sequence Identity. Sequence identity is determined by comparing a reference sequence or a subsequence of the reference sequence to a test sequence (e.g., a nucleotide sequence, an amino acid sequence, etc.). The reference sequence and the test sequence are optimally aligned over an arbitrary number of residues termed a comparison window. In order to obtain optimal alignment, additions or deletions, such as gaps, may be introduced into the test sequence. The percent sequence identity is determined by determining the number of positions at which the same residue is present in both sequences and dividing the number of matching positions by the total length of the sequences in the comparison window and multiplying by 100 to give the percentage. In addition to the number of matching positions, the number and size of gaps is also considered in calculating the percentage sequence identity.

Sequence identity is typically determined using computer programs. A representative program is the BLAST (Basic Local Alignment Search Tool) program publicly accessible at the National Center for Biotechnology Information (NCBI, http://www.ncbi.nlm.nih.gov/). This program compares segments in a test sequence to sequences in a database to determine the statistical significance of the matches, then identifies and reports only those matches that that are more significant than a threshold level. A suitable version of the BLAST program is one that allows gaps, for example, version 2.X (Altschul, et al., Nucleic Acids Res 25(17):3389-402, 1997). Standard BLAST programs for searching nucleotide sequences (blastn) or protein (blastp) may be used. Translated query searches in which the query sequence is translated, i.e., from nucleotide sequence to protein (blastx) or from protein to nucleic acid sequence (tbblastn) may also be used as well as queries in which a nucleotide query sequence is translated into protein sequences in all 6 reading frames and then compared to an NCBI nucleotide database which has been translated in all six reading frames may be used (tbblastx).

Additional suitable programs for identifying proteins with sequence identity to the proteins of the invention include, but are not limited to, PHI-BLAST (Pattern Hit Initiated BLAST, Zhang, et al., Nucleic Acids Res 26(17):3986-90, 1998) and PSI-BLAST (Position-Specific Iterated BLAST, Altschul, et al., Nucleic Acids Res 25(17):3389-402, 1997).

Programs may be used with default searching parameters. Alternatively, one or more search parameter may be adjusted. Selecting suitable search parameter values is within the abilities of one of ordinary skill in the art.

### 1. Polypeptides of the Invention

In one aspect, the present invention provides polypeptides having a DNA polymerase activity (e.g., a DNA-dependent DNA polymerase activity and/or an RNA-dependent DNA polymerase activity). Polypeptides of the invention may preferably possess an RNA-dependent DNA polymerase activity, which may be active in the presence of Mg²⁺. Polypeptides of the invention may possess, or may not possess, one or more enzymatic activities in addition to DNA polymerase activities. For example, polypeptides of the invention may possess, or may not possess, an exonuclease activity (e.g., 5′-3′ exonuclease activity and/or 3′-5′ exonuclease activity). Preferably, polypeptides of the invention may be purified and/or isolated from a cell or organism expressing them, which may be a wild type cell or organism or a recombinant cell or organism. In some embodiments, such polypeptides may be substantially isolated from the cell or organism in which they are expressed. In some embodiments, polypeptides of the invention may be substantially pure.

In some embodiments, the polypeptide may be a DNA polymerase from a thermophilic eubacterium. Suitable eubacteria include, but are not limited to, Clostridium spp. (e.g., Clostridium stercorarium, Clostridium thermosulfurogenes, etc.), Caldibacillus spp. (e.g., Caldibacillus cellulovorans CompA.2), Caldicellulosiruptor spp. (e.g., Caldicellulosiruptor Tok13B, Caldicellulosiruptor Tok7B, Caldicellulosiruptor RT69B), Bacillus spp. (e.g., Bacillus caldolyticus EA1), Thermus spp. (e.g., Thermus RT41A), Dictyoglomus spp. (e.g., Dictyoglomus thermophilum), Spirochaete spp., and Tepidomonas spp. Polymerases can be isolated from any suitable strain of thermophilic eubacteria. Preferred thermophilic eubacterial strains from which to isolate a nucleic acid encoding DNA polymerase of the invention include those listed above. SEQ ID NOS: 2-13 are DNA sequences encoding a representative number of the polypeptides of the invention and the corresponding amino acid sequences are provided by SEQ ID NOS: 14-25. SEQ ID NOS: 27-34 are sequences of a variety of eubacterial DNA polymerases.

Polypeptides of the invention preferably possess an RNA-dependent DNA polymerase activity (i.e., a reverse transcriptase activity). This activity preferably occurs in the presence of Mg²⁺ as a divalent metal cofactor and, in some embodiments, this activity does not require the presence of any additional divalent metal ion cofactors (e.g. does not require the presence of an error-inducing metal such as Mn²⁺).

Those skilled in the art will recognize that several of the sequences of the polypeptides of the invention are provided with N-terminal tag sequences (e.g., a PelB leader) that are a result of the particular vector into which the coding sequence of the polypeptide was inserted. The amino acid sequences of a representative number of the polypeptides of the invention are provided in SEQ ID NOS: 14-25. Those skilled in the art will appreciate that the sequences provided include the leader sequences derived from the vector. In the interest of clarity of numbering of amino acid residues, numbers provided herein will include any leader sequence.

It has been unexpectedly found that the presence of one or more sequence motifs in a polypeptide of the invention is associated with the ability of the polypeptide to perform RNA-dependent DNA polymerase activity. The present invention identifies the Q-helix as a sequence motif associated with Mg²⁺ dependent RT activity and identifies specified amino acid residues within the Q-helix as being particularly important in assessing the potential for reverse transcriptase activity. A representative Q-helix may have the sequence RY—X₈—Y—X₃—SFAER, (SEQ ID NO:1) wherein X is any imino or amino acid. Other representative Q-helices (see Tables 35 and 37) include amino acid numbers 823 to 842 of the sequence of E. coli DNA polymerase I (SEQ ID NO:34), amino acid numbers 728 to 747 of Thermus aquaticus (Taq) DNA polymerase (SEQ ID NO:27), and amino acid numbers 820-838 of the Caldibacillus cellulovorans CompA.2 DNA polymerase amino acid sequence of SEQ ID NO:16. Each X may independently represent an Ala, Cys, Asp, Glu, Phe, Gly, His, Ile, Lys, Leu, Met, Asn, Pro, Gln, Arg, Ser, Thr, Val, Trp, or Tyr or may represent an amino or imino acid that is not naturally produced in most host cells. Q-helix motifs associated with Mg²⁺ dependent RT activity include, but are not limited to, Q-helices wherein position 11 of the Q-helix (SEQ ID NO:1) may be a phenylalanine or a tyrosine (F or Y) independently of the amino acid residue at positions 15 and/or 16. In some embodiments, position 15 of the Q-helix (SEQ ID NO:1) may be a serine or asparagine (S or N) independently of the amino acid residue at positions 11 and/or 16. In some embodiments, position 16 of the Q-helix (SEQ ID NO:1) may be a tyrosine or phenylalanine (Y or F) independently of the amino acid residue at positions 11 and/or 12. In one embodiment, position 11 may be a phenylalanine residue while position 15 is a serine residue and position 16 is a phenylalanine.

In another aspect, polypeptides of the invention include those with one or more specified amino acid residues at positions that correspond to Q628, 1659, Q668, F669 and/or Q753 of the Caldibacillus cellulovorans CompA.2 (CompA.2) DNA polymerase amino acid sequence presented in SEQ ID NO:16. In some embodiments, polypeptides of the invention may include a residue at a position that corresponds to position 628 that is not a lysine or glutamate residue. Suitable amino acid residues include Ala, Cys, Asp, Phe, Gly, His, Ile, Leu, Met, Asn, Pro, Gln, Arg, Ser, Thr, Val, Trp, or Tyr. In some embodiments, polypeptides of the invention may have a glutamine residue at a position corresponding to position 628 of the ComA2 polymerase. In some embodiments, polypeptides of the invention may include a residue at a position corresponding to 1659 of the CompA.2 DNA polymerase that is not a glycine. Suitable residues include Ala, Cys, Asp, Glu, Phe, His, Ile, Lys, Leu, Met, Asn, Pro, Gln, Arg, Ser, Thr, Val, Trp, or Tyr or may be an amino or imino acid that is not naturally produced in most host cells. In some embodiments, polypeptides of the invention may have a hydrophobic residue at this position, for example, Ile, Val, and/or Leu. In some embodiments, polypeptides of the invention may include a residue at a position corresponding to Q668 of the CompA.2 DNA polymerase that is not a serine. Suitable residues include Ala, Cys, Asp, Glu, Phe, Gly, His, Ile, Lys, Leu, Met, Asn, Pro, Gln, Arg, Thr, Val, Trp, or Tyr or may be an amino or imino acid that is not naturally produced in most host cells. In some embodiments, polypeptides of the invention may have a glutamine and/or a threonine at this position. In some embodiments, polypeptides of the invention may include a residue at a position corresponding to F669 of the CompA.2 DNA polymerase that is not an aspartate or glutamate. Suitable residues include Ala, Cys, Phe, Gly, His, Ile, Lys, Leu, Met, Asn, Pro, Gln, Arg, Ser, Thr, Val, Trp, or Tyr or may be an amino or imino acid that is not naturally produced in most host cells. In some embodiments, polypeptides of the invention may have an aromatic amino acid at this position, for example, a phenylalanine. In some embodiments, polypeptides of the invention may include a residue at a position corresponding to Q753 of the CompA.2 DNA polymerase that is not an alanine or valine. Suitable residues include Cys, Asp, Glu, Phe, Gly, His, Ile, Lys, Leu, Met, Asn, Pro, Gln, Arg, Ser, Thr, Trp, or Tyr or may be an amino or imino acid that is not naturally produced in most host cells. In some embodiments, polypeptides of the invention may have a glutamine at this position.

Some or all of the polypeptides of the invention may possess an RNA-dependent DNA polymerase activity. Mutants may be made of the polypeptides of the invention that have an enhanced RNA-dependent DNA polymerase activity as compared to the wild type polypeptide of the invention. Alternatively, for those polypeptides of the invention that lack a detectable RNA-dependent DNA polymerase activity, mutants having such activity may be constructed according to the present invention. The present invention provides amino acid residues associated with reverse transcriptase activity in eubacterial DNA polymerases. Such reverse transcriptase activity is preferably observed in the presence of Mg²⁺ as a divalent cation, optionally in the absence of Mn²⁺.

Mutants having an enhanced reverse transcriptase activity are preferably constructed by mutating one or more amino acids of the Q-helix of the polymerase. The Q-helix is defined as RY—X₈—Y—X₃—SFAER, (SEQ ID NO:1) wherein X is any imino or amino acid. Representative Q-helices include amino acid numbers 823 to 842 of the sequence of E. coli DNA polymerase I, amino acid numbers 728 to 747 of Thermus aquaticus (Taq) DNA polymerase, and amino acid numbers 820-838 of the Caldibacillus cellulovorans CompA.2 DNA polymerase amino acid sequence presented in SEQ ID NO:16. Tables 35 and 37 provide the location and sequence of a representative number of Q-helices from a variety of eubacterial DNA polymerases. Each X may independently represent an Ala, Cys, Asp, Glu, Phe, Gly, His, Ile, Lys, Leu, Met, Asn, Pro, Gln, Arg, Ser, Thr, Val, Trp, or Tyr or may represent an amino or imino acid that is not naturally produced in most host cells. Each X can be determined by selecting a corresponding nucleic acid codon. Modified or natural tRNAs can be used to introduce specific amino acids into the sequence at any X. In some preferred embodiments, position 11 of the Q-helix (SEQ ID NO:1) may be a phenylalanine or a tyrosine (F or Y) independently of the amino acid residue at positions 15 and/or 16. In some embodiments, position 15 of the Q-helix (SEQ ID NO:1) may be a serine or asparagine (S or N) independently of the amino acid residue at positions 11 and/or 16. In some embodiments, position 16 of the Q-helix (SEQ ID NO:1) may be a tyrosine or phenylalanine (Y or F) independently of the amino acid residue at positions 11 and/or 12. In one embodiment, position 11 of the Q-helix may be a phenylalanine residue while position 15 is a serine residue and position 16 is a phenylalanine.

In some embodiments, the present invention provides mutant DNA polymerases derived from eubacterial DNA polymerases. Preferably, such mutants may have an increased RNA-dependent DNA polymerase activity as compared to the wildtype polymerase (e.g., in the presence of Mg²⁺). In some embodiments, such mutants may have one or more mutations in the amino acid sequence of the Q-helix. Preferred mutations include changing an amino acid at position 11 of the Q-helix to phenylalanine or tyrosine (F or Y), changing an amino acid at position 15 of the Q-helix to serine or asparagine (S or N), and/or changing an amino acid at position 16 of the Q-helix to tyrosine or phenylalanine (Y of F). Mutants may comprise one or more of these mutations. In one embodiment, mutants may comprise a phenylalanine at position 11, a serine at position 15, and a phenylalanine at position 16.

If the polypeptide of the invention has 3′-to-5′ exonuclease activity, this activity may be reduced, substantially reduced, or eliminated by mutating the gene encoding the polypeptide. Such mutations include point mutations, frame shift mutations, deletions and/or insertions. Preferably, the region of the gene encoding the 3′-to-5′ exonuclease activity is mutated or deleted using techniques well known in the art (for example Sambrook, et al, (1989) in: Molecular Cloning, A Laboratory Manual (2nd Ed.), Cold Spring Harbor Laboratory Press, Cold Spring Harbor, N.Y.).

5′-to-3′ exonuclease activity of a polypeptide of the invention can likewise be reduced, substantially reduced, or eliminated by mutating the gene encoding the polypeptide. Such mutations include point mutations, frame shift mutations, deletions, and/or insertions. Preferably, the region of the gene encoding the 5′-to-3′ exonuclease activity is deleted using techniques well known in the art. In specific embodiments of this invention, any conserved amino acids that are associated with the 5′-to-3′ exonuclease activity can be mutated. Examples of these conserved amino acids are amino acids that correspond to Asp8, Lys77, Glu112, Asp114, Asp115, Asp137, Asp139, or Lys202 of Thermatoga neopolitina DNA polymerase, which correspond to Asp32, Lys97, Glu132, Asp134, Asp135, Asp157, Asp159, or Lys222 of the Caldibacillus cellulovorans CompA.2 DNA polymerase.

The present invention is directed broadly to mutations of DNA polymerases that result in the reduction or elimination of 5′-3′ exonuclease activity. Other particular mutations correspond to the following amino acids.

E. coli PolI: Asp13, Glu113, Asp115, Asp116, Asp138, and Asp140.

Taq Pol: Asp18, Glu117, Asp 119, Asp120, Asp142, and Asp144.

Tma Pol: Asp8, Glu112, Asp114, Asp115, Asp137, and Asp139. Amino acid residues of Taq DNA polymerase are as numbered in U.S. Pat. No. 5,079,352 and SEQ ID NO:27. Amino acid residues of Thermotoga maritima (Tma) DNA polymerase are numbered as in U.S. Pat. No. 5,374,553.

By comparison to the amino acid sequence of other DNA polymerases, the corresponding sites can easily be located in the polypeptides of the invention and the DNA altered to produce a coding sequence for a mutated polypeptide of the invention that lacks 5′-3′ exonuclease activity. Examples of suitable sites in the polypeptides of the invention to be mutated include those corresponding to the following sites in other DNA polymerases:

| Enzyme or source | Mutation positions |
|---|---|
| Streptococcus pneumoniae | Asp10, Glu114, Asp16, Asp117, Asp139, |
| | Asp141 |
| Thermus flavus | Asp17, Glu116, Asp118, Asp119, |
| | Asp141, Asp143 |
| Thermus thermophilus | Asp18, Glu118, Asp120, Asp121, |
| | Asp143, Asp145 |
| Deinococcus radiodurans | Asp18, Glu117, Asp119, Asp120, |
| | Asp142, Asp144 |
| Bacillus caldotenax | Asp9, Glu109, Asp111, Asp112, Asp134, |
| | Asp136 |

Coordinates of S. pneumoniae, T. flavus, D. radiodurans, B. caldotenax were obtained from Gutman and Minton, supra. Coordinates of T. thermophilus were obtained from International Patent No. WO 92/06200. The sequences of a representative number of the polypeptides of the invention may be aligned one skilled in the art can readily identify the corresponding residues in the polypeptides of the invention by consulting the alignment.

To abolish 5′-3′ exonuclease activity, amino acids are preferably selected to have different properties. For example, an acidic amino acid such as Asp or Glu may be changed to a basic, neutral or polar but uncharged amino acid such as Lys, Arg, His (basic); Ala, Val, Leu, Ile, Pro, Met, Phe, Trp (neutral); or Gly, Ser, Thr, Cys, Tyr, Asn or Gln (polar but uncharged). For example, Glu may be changed to Asp, Ala, Val Leu, Ile, Pro, Met, Phe, Trp, Gly, Ser, Thr, Cys, Tyr, Asn or Gln. Specifically, the Ala substitution in the corresponding position of an acid residue is expected to abolish 5′-3′ exonuclease activity.

In a preferred embodiment, oligonucleotide directed mutagenesis is used to create mutant polypeptides of the invention. This allows for all possible base pair changes at any determined site along the encoding DNA molecule. In general, this technique involves annealing an oligonucleotide complementary (except for one or more desired mismatches) to a single stranded nucleotide sequence coding for the native DNA polymerase of interest. The mismatched oligonucleotide is then extended by DNA polymerase, generating a double stranded DNA molecule which contains the desired change in sequence on one strand. The changes in sequence can of course result in the deletion, substitution, or insertion of an amino acid. The changed strand can be used as a template to form a double stranded polynucleotide. The double stranded polynucleotide can then be inserted into an appropriate expression vector, and a mutant polypeptide can thus be produced. The above-described oligonucleotide directed mutagenesis can be carried out using any technique known to those skilled in the art, for example, PCR. Preferably, mutations designed to alter the exonuclease activity do not adversely affect the polymerase activity.

In other embodiments, the entire 5′-to-3′ exonuclease domain of a DNA polymerase can be deleted by proteolytic cleavage or by genetic engineering. For example, a unique restriction site can be used to obtain a clone devoid of nucleotides encoding the amino terminal amino acids of DNA polymerase associated with the activity (e.g., amino acids 1 to about 304 of the Caldibacillus cellulovorans CompA.2 sequence presented in SEQ ID NO:16). Alternatively, less than the entire amino terminal domain may be removed, for example, by treating the DNA coding for the eubacterial DNA polymerase with an exonuclease, isolating the fragments, ligating the fragments into a cloning vehicle, transfecting cells with the cloning vehicle, and screening the transformants for DNA polymerase activity and lack of 5′-to-3′ exonuclease activity. These tasks may be accomplished by one skilled in the art with no more than routine experimentation.

Mutations may be made in the polypeptides of the invention to render them less discriminating or non-discriminating against non-natural nucleotides such as dideoxynucleotides. Changes within the O-helix of the polypeptides of the invention, such as other point mutations, deletions, and insertions, can be made to render the polymerase non-discriminating. The O-helix region is a 14 amino acid sequence corresponding to amino acids 746-759 of the Clostridium stercorarium sequence presented in SEQ ID NO:14 and amino acid numbers 751-764 of the Caldibacillus cellulovorans CompA.2 sequence presented in SEQ ID NO:16. The O-helix may be defined as RXXXKXXXFXXXYX, (SEQ ID NO:26) wherein X is any amino acid. The most important amino acids in conferring discriminatory activity include Arg, Lys and Phe (R746, K750, F754 in SEQ ID NO:14 and R751, K755, and F759 in SEQ ID NO:16). With reference to the sequence SEQ ID NO: 14, amino acids which may be substituted for Arg at position 746 (and in the corresponding position of other polypeptides of the invention) include Asp, Glu, Ala, Val Leu, Ile, Pro, Met, Phe, Trp, Gly, Ser, Thr, Cys, Tyr, Gln, Asn, Lys and His or other less common natural or unnatural amino acids. Amino acids that may be substituted for Phe at position 754 (and in the corresponding position of other polypeptides of the invention) include Lys, Arg, His, Asp, Glu, Ala, Val, Leu, Ile, Pro, Met, Trp, Gly, Ser, Thr, Cys, Tyr, Asn and Gln or other less common natural or unnatural amino acids. Amino acids that may be substituted for Lys at position 750 (and in the corresponding position of other polypeptides of the invention) include Tyr, Arg, His, Asp, Glu, Ala, Val, Leu, Ile, Pro, Met, Trp, Gly, Ser, Thr, Cys, Phe, Asn and Gln or other less common natural or unnatural amino acids. Preferred mutants include Tyr754, Ala754, Ser754 and Thr754. Any of the one or more of the amino acids conferring discriminatory activity may be substituted to alter discrimination. Such mutants may be prepared by well known methods of site directed mutagenesis known in the art or as described herein. Other amino acids such as ornithine can be substituted for any one or more of the amino acids conferring discriminatory activity. For example, unnatural tRNAs can be used to insert other amino acids.

Polypeptides of the invention include, but are not limited to, polypeptides comprising, or alternatively consisting of, an amino acid sequence selected from SEQ ID NOS:14-25, polypeptides comprising, or alternatively consisting of, a polypeptide encoded by a nucleotide sequence selected from SEQ ID NOS:2-13, polypeptides comprising, or alternatively consisting of, a polypeptide encoded by a nucleotide sequence of one of the deposited clones (NRRL Deposit Numbers NRRL B-30617, NRRL B-30618, NRRL B-30619, NRRL B-30620, NRRL B-30621, NRRL B-30622, NRRL B-30623, NRRL B-30624, NRRL B-30625, NRRL B-30626, NRRL B-30576, NRRL B-30577, NRRL B-30579, NRRL B-30578, NRRL B-30580), and/or mutants, fragments (e.g., portions), and variants thereof. As described below, the invention also includes polynucleotides encoding such polypeptides.

As described above, and further described below, polypeptides of the invention also include, but are not limited to, polypeptides comprising, or alternatively consisting of, mutant polymerases which comprise one or more substitutions corresponding to an amino acid residue of an amino acid sequence selected from SEQ ID NOS:14-25, polypeptides comprising, or alternatively consisting of, mutant polymerases which comprise one or more substitutions (e.g., one, two, three, four, five, six, seven, eight, nine, ten, etc.) corresponding to an amino acid residue of a polypeptide encoded by a nucleotide sequence selected from SEQ ID NOS: 2-13, polypeptides comprising, or alternatively consisting of, mutant polymerases which comprise one or more substitutions (e.g., one, two, three, four, five, six, seven, eight, nine, ten, etc.) corresponding to an amino acid residue of a polypeptide encoded by a nucleotide sequence of one of the deposited clones (NRRL Deposit Numbers NRRL B-30617, NRRL B-30618, NRRL B-30619, NRRL B-30620, NRRL B-30621, NRRL B-30622, NRRL B-30623, NRRL B-30624, NRRL B-30625, NRRL B-30626, NRRL B-30576, NRRL B-30577, NRRL B-30579, NRRL B-30578, NRRL B-30580), and/or mutants, fragments (e.g., portions), and variants thereof. As described below, the invention also includes polynucleotides encoding such polypeptides.

The nucleotide sequences of SEQ ID NOS: 2-13 and the translated corresponding amino acid sequences of SEQ ID NOS:14-25, are sufficiently accurate and otherwise suitable for a variety of uses well known in the art and described further below. For instance, the nucleotide sequences of SEQ ID NOS:2-13 are useful for designing nucleic acid hybridization probes/primers that will detect and/or amplify nucleic acid sequences contained in SEQ ID NOS: 2-13, respectively, or the DNAs contained in the respective deposited clone. These probes/primers will also hybridize to/amplify nucleic acid molecules in microbiological samples, thereby enabling detection of the respective organism from which SEQ ID NOS: 2-13 are derived. Similarly, polypeptides identified from SEQ ID NOS:14-25 may be used, for example, to generate antibodies which bind specifically to the polypeptides of the invention.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequences SEQ ID NOS: 2-13 and the corresponding predicted translated amino acid sequences of SEQ ID NOS:14-25, but also a sample of plasmid DNA containing a DNA clone encoding the polymerases of the invention deposited with the NRRL (see examples). The nucleotide sequence of the deposited clones can readily be determined by sequencing the deposited clones in accordance with known methods. The predicted amino acid sequences can then be verified from such deposits. Moreover, the amino acid sequence of the protein encoded by the deposited clone can also be directly determined by peptide sequencing or by expressing the protein in a suitable host cell containing the deposited DNA, collecting the protein, and determining its sequence.

Polypeptides of the invention include polypeptides comprising or consisting of fragments of the polypeptides of SEQ ID NOS:14-25, preferably fragments of the polymerases of SEQ ID NOS:14-25 (i.e., the polypeptides set out in these sequences which do not contain the N-terminal amino acids encoded by the vector nucleic acids (e.g., the first 22 amino acids set out in SEQ ID NO:14)) and fragments of the polymerases encoded by the deposited clones. Polypeptide fragments of the invention may be employed for producing the corresponding full-length polypeptide by peptide synthesis, therefore, the fragments may be employed as intermediates for producing the full-length polypeptides. Polypeptide fragments of the invention may also be employed for generating antibody, as described herein.

Polypeptide fragments of the invention may be from 6 to 959 amino acids in length. In many instances, these polypeptides fragments comprise or consist of amino acid sequences set out in one or more of SEQ ID NOS:14-25 with or without the N-terminal amino acids encoded by the vectors (i.e., fragments of the full-length polypeptide or the polymerase set out in SEQ ID NOS:14-25).

Polypeptide fragments of the invention may be, for example, at least 10 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 10 amino acid long fragments including amino acid residues 1-10, 2-11, 3-12, . . . , 911-920 of the polypeptide or polymerase of SEQ ID NO:14; residues 1-10, 2-11, 3-12, . . . , 880-889 of the polypeptide or polymerase of SEQ ID NO:15; residues 1-10, 2-11, 3-12, . . . , 916-925 of the polypeptide or polymerase of SEQ ID NO:16; residues 1-10, 2-11, 3-12, . . . , 862-871 of the polypeptide or polymerase of SEQ ID NO:17; residues 1-10, 2-11, 3-12, . . . , 862-871 of the polypeptide or polymerase of SEQ ID NO:18; residues 1-10, 2-11, 3-12, . . . , 862-871 of the polypeptide or polymerase of SEQ ID NO:19; residues 1-10, 2-11, 3-12, . . . , 891-900 of the polypeptide or polymerase of SEQ ID NO:20; residues 1-10, 2-11, 3-12, . . . , 855-864 of the polypeptide or polymerase of SEQ ID NO:21; residues 1-10, 2-11, 3-12, . . . , 875-884 of the polypeptide or polymerase of SEQ ID NO:22; residues 1-10, 2-11, 3-12, . . . , 861-870 of the polypeptide or polymerase of SEQ ID NO:23; residues 1-10, 2-11, 3-12, . . . , 919-928 of the polypeptide or polymerase of SEQ ID NO:24; residues 1-10, 2-11, 3-12, . . . , 951-960 of the polypeptide or polymerase of SEQ ID NO:25.

Polypeptide fragments of the invention may be at least 11 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 11 amino acid long fragments including amino acid residues 1-11, 2-12, 3-13, . . . , 910-920 of the polypeptide or polymerase of SEQ ID NO:14; residues 1-11, 2-12, 3-13, . . . , 879-889 of the polypeptide or polymerase of SEQ ID NO:15; residues 1-11, 2-12, 3-13, . . . , 915-925 of the polypeptide or polymerase of SEQ ID NO:16; residues 1-11, 2-12, 3-13, . . . , 861-871 of the polypeptide or polymerase of SEQ ID NO:17; residues 1-11, 2-12, 3-13, . . . , 861-871 of the polypeptide or polymerase of SEQ ID NO:18; residues 1-11, 2-12, 3-13, . . . , 861-871 of the polypeptide or polymerase of SEQ ID NO:19; residues 1-11, 2-12, 3-13, . . . , 890-900 of the polypeptide or polymerase of SEQ ID NO:20; residues 1-11, 2-12, 3-13, . . . , 854-864 of the polypeptide or polymerase of SEQ ID NO:21; residues 1-11, 2-12, 3-13, . . . , 874-884 of the polypeptide or polymerase of SEQ ID NO:22; residues 1-11, 2-12, 3-13, . . . , 860-870 of the polypeptide or polymerase of SEQ ID NO:23; residues 1-11, 2-12, 3-13, . . . , 918-928 of the polypeptide or polymerase of SEQ ID NO:24; residues 1-11, 2-12, 3-13, . . . , 950-960 of the polypeptide or polymerase of SEQ ID NO:25.

Polypeptide fragments of the invention may be at least 12 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 12 amino acid long fragments including amino acid residues 1-12, 2-13, 3-14, . . . , 909-920 of the polypeptide or polymerase of SEQ ID NO:14; residues 1-12, 2-13, 3-14, . . . , 878-889 of the polypeptide or polymerase of SEQ ID NO:15; residues 1-12, 2-13, 3-14, . . . , 914-925 of the polypeptide or polymerase of SEQ ID NO:16; residues 1-12, 2-13, 3-14, . . . , 860-871 of the polypeptide or polymerase of SEQ ID NO:17; residues 1-12, 2-13, 3-14, . . . , 860-871 of the polypeptide or polymerase of SEQ ID NO:18; residues 1-12, 2-13, 3-14, . . . , 860-871 of the polypeptide or polymerase of SEQ ID NO:19; residues 1-12, 2-13, 3-14, . . . , 889-900 of the polypeptide or polymerase of SEQ ID NO:20; residues 1-12, 2-13, 3-14, . . . , 853-864 of the polypeptide or polymerase of SEQ ID NO:21; residues 1-12, 2-13, 3-14, . . . , 873-884 of the polypeptide or polymerase of SEQ ID NO:22; residues 1-12, 2-13, 3-14, . . . , 859-870 of the polypeptide or polymerase of SEQ ID NO:23; residues 1-12, 2-13, 3-14, . . . , 917-928 of the polypeptide or polymerase of SEQ ID NO:24; residues 1-12, 2-13, 3-14, . . . , 949-960 of the polypeptide or polymerase of SEQ ID NO:25.

Polypeptide fragments of the invention may be at least 13 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 13 amino acid long fragments including amino acid residues 1-13, 2-14, 3-15, . . . , 908-920 of the polypeptide or polymerase of SEQ ID NO:14; residues 1-13, 2-14, 3-15, . . . , 877-889 of the polypeptide or polymerase of SEQ ID NO:15; residues 1-13, 2-14, 3-15, . . . , 913-925 of the polypeptide or polymerase of SEQ ID NO:16; residues 1-13, 2-14, 3-15, . . . , 859-871 of the polypeptide or polymerase of SEQ ID NO:17; residues 1-13, 2-14, 3-15, . . . , 859-871 of the polypeptide or polymerase of SEQ ID NO:18; residues 1-13, 2-14, 3-15, . . . , 859-871 of the polypeptide or polymerase of SEQ ID NO:19; residues 1-13, 2-14, 3-15, . . . , 888-900 of the polypeptide or polymerase of SEQ ID NO:20; residues 1-13, 2-14, 3-15, . . . , 852-864 of the polypeptide or polymerase of SEQ ID NO:21; residues 1-13, 2-14, 3-15, . . . , 872-884 of the polypeptide or polymerase of SEQ ID NO:22; residues 1-13, 2-14, 3-15, . . . , 858-870 of the polypeptide or polymerase of SEQ ID NO:23; residues 1-13, 2-14, 3-15, . . . , 916-928 of the polypeptide or polymerase of SEQ ID NO:24; residues 1-13, 2-14, 3-15, . . . , 948-960 of the polypeptide or polymerase of SEQ ID NO:25.

Polypeptide fragments of the invention may be at least 14 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 14 amino acid long fragments including amino acid residues 1-14, 2-15, 3-16, . . . , 907-920 of the polypeptide or polymerase of SEQ ID NO:14; residues 1-14, 2-15, 3-16, . . . , 876-889 of the polypeptide or polymerase of SEQ ID NO:15; residues 1-14, 2-15, 3-16, . . . , 912-925 of the polypeptide or polymerase of SEQ ID NO:16; residues 1-14, 2-15, 3-16, . . . , 858-871 of the polypeptide or polymerase of SEQ ID NO:17; residues 1-14, 2-15, 3-16, . . . , 858-871 of the polypeptide or polymerase of SEQ ID NO:18; residues 1-14, 2-15, 3-16, . . . , 858-871 of the polypeptide or polymerase of SEQ ID NO:19; residues 1-14, 2-15, 3-16, . . . , 887-900 of the polypeptide or polymerase of SEQ ID NO:20; residues 1-14, 2-15, 3-16, . . . , 851-864 of the polypeptide or polymerase of SEQ ID NO:21; residues 1-14, 2-15, 3-16, . . . , 871-884 of the polypeptide or polymerase of SEQ ID NO:22; residues 1-14, 2-15, 3-16, . . . , 857-870 of the polypeptide or polymerase of SEQ ID NO:23; residues 1-14, 2-15, 3-16, . . . , 915-928 of the polypeptide or polymerase of SEQ ID NO:24; residues 1-14, 2-15, 3-16, . . . , 947-960 of the polypeptide or polymerase of SEQ ID NO:25.

Polypeptide fragments of the invention may be at least 15 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 15 amino acid long fragments including amino acid residues 1-15, 2-16, 3-17, . . . , 906-920 of the polypeptide or polymerase of SEQ ID NO:14; residues 1-15, 2-16, 3-17, . . . , 875-889 of the polypeptide or polymerase of SEQ ID NO:15; residues 1-15, 2-16, 3-17, . . . , 911-925 of the polypeptide or polymerase of (SEQ ID NO:16; residues 1-15, 2-16, 3-17, . . . , 857-871 of the polypeptide or polymerase of SEQ ID NO:17; residues 1-15, 2-16, 3-17, . . . , 857-871 of the polypeptide or polymerase of SEQ ID NO:18; residues 1-15, 2-16, 3-17, . . . , 857-871 of the polypeptide or polymerase of SEQ ID NO:19; residues 1-15, 2-16, 3-17, . . . , 886-900 of the polypeptide or polymerase of SEQ ID NO:20; residues 1-15, 2-16, 3-17, . . . , 850-864 of the polypeptide or polymerase of SEQ ID NO:21; residues 1-15, 2-16, 3-17, . . . , 870-884 of the polypeptide or polymerase of SEQ ID NO:22; residues 1-15, 2-16, 3-17, . . . , 856-870 of the polypeptide or polymerase of SEQ ID NO:23; residues 1-15, 2-16, 3-17, . . . , 914-928 of the polypeptide or polymerase of SEQ ID NO:24; residues 1-15, 2-16, 3-17, . . . , 946-960 of the polypeptide or polymerase of SEQ ID NO:25.

Polypeptide fragments of the invention may be at least 16 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 16 amino acid long fragments including amino acid residues 1-16, 2-17, 3-18, . . . , 905-920 of the polypeptide or polymerase of SEQ ID NO:14; residues 1-16, 2-17, 3-18, . . . , 874-889 of the polypeptide or polymerase of SEQ ID NO:15; residues 1-16, 2-17, 3-18, . . . , 910-925 of the polypeptide or polymerase of SEQ ID NO:16; residues 1-16, 2-17, 3-18, . . . , 856-871 of the polypeptide or polymerase of SEQ ID NO:17; residues 1-16, 2-17, 3-18, . . . , 856-871 of the polypeptide or polymerase of SEQ ID NO:18; residues 1-16, 2-17, 3-18, . . . , 856-871 of the polypeptide or polymerase of SEQ ID NO:19; residues 1-16, 2-17, 3-18, . . . , 885-900 of the polypeptide or polymerase of SEQ ID NO:20; residues 1-16, 2-17, 3-18, . . . , 849-864 of the polypeptide or polymerase of SEQ ID NO:21; residues 1-16, 2-17, 3-18, . . . , 869-884 of the polypeptide or polymerase of SEQ ID NO:22; residues 1-16, 2-17, 3-18, . . . , 855-870 of the polypeptide or polymerase of SEQ ID NO:23); residues 1-16, 2-17, 3-18, . . . , 913-928 of the polypeptide or polymerase of (SEQ ID NO:24; residues 1-16, 2-17, 3-18, . . . , 945-960 of the polypeptide or polymerase of SEQ ID NO:25.

Polypeptide fragments of the invention may be at least 17 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 17 amino acid long fragments including amino acid residues 1-17, 2-18, 3-19, . . . , 904-920 of the polypeptide or polymerase of SEQ ID NO:14; residues 1-17, 2-18, 3-19, . . . , 873-889 of the polypeptide or polymerase of SEQ ID NO:15; residues 1-17, 2-18, 3-19, . . . , 909-925 of the polypeptide or polymerase of SEQ ID NO:16; residues 1-17, 2-18, 3-19, . . . , 855-871 of the polypeptide or polymerase of SEQ ID NO:17; residues 1-17, 2-18, 3-19, . . . , 855-871 of the polypeptide or polymerase of SEQ ID NO:18; residues 1-17, 2-18, 3-19, . . . , 855-871 of the polypeptide or polymerase of SEQ ID NO:19; residues 1-17, 2-18, 3-19, . . . , 884-900 of the polypeptide or polymerase of SEQ ID NO:20; residues 1-17, 2-18, 3-19, . . . , 848-864 of the polypeptide or polymerase of SEQ ID NO:21; residues 1-17, 2-18, 3-19, . . . , 868-884 of the polypeptide or polymerase of SEQ ID NO:22; residues 1-17, 2-18, 3-19, . . . , 854-870 of the polypeptide or polymerase of SEQ ID NO:23; residues 1-17, 2-18, 3-19, . . . , 912-928 of the polypeptide or polymerase of SEQ ID NO:24; residues 1-17, 2-18, 3-19, . . . , 944-960 of the polypeptide or polymerase of SEQ ID NO:25.

Polypeptide fragments of the invention may be at least 18 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 18 amino acid long fragments including amino acid residues 1-18, 2-19, 3-20, . . . , 903-920 of the polypeptide or polymerase of SEQ ID NO:14; residues 1-18, 2-19, 3-20, . . . , 872-889 of the polypeptide or polymerase of SEQ ID NO:15; residues 1-18, 2-19, 3-20, . . . , 908-925 of the polypeptide or polymerase of SEQ ID NO:16; residues 1-18, 2-19, 3-20, . . . , 854-871 of the polypeptide or polymerase of SEQ ID NO:17; residues 1-18, 2-19, 3-20, . . . , 854-871 of the polypeptide or polymerase of SEQ ID NO:18; residues 1-18, 2-19, 3-20, . . . , 854-871 of the polypeptide or polymerase of SEQ ID NO:19; residues 1-18, 2-19, 3-20, . . . , 883-900 of the polypeptide or polymerase of SEQ ID NO:20; residues 1-18, 2-19, 3-20, . . . , 847-864 of the polypeptide or polymerase of SEQ ID NO:21; residues 1-18, 2-19, 3-20, . . . , 867-884 of the polypeptide or polymerase of SEQ ID NO:22; residues 1-18, 2-19, 3-20, . . . , 853-870 of the polypeptide or polymerase of SEQ ID NO:23; residues 1-18, 2-19, 3-20, . . . , 911-928 of the polypeptide or polymerase of SEQ ID NO:24; residues 1-18, 2-19, 3-20, . . . , 943-960 of the polypeptide or polymerase of SEQ ID NO:25.

Polypeptide fragments of the invention may be at least 19 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 19 amino acid long fragments including amino acid residues 1-19, 2-20, 3-21, . . . , 902-920 of the polypeptide or polymerase of SEQ ID NO:14; residues 1-19, 2-20, 3-21, . . . , 871-889 of the polypeptide or polymerase of SEQ ID NO:15; residues 1-19, 2-20, 3-21, . . . , 907-925 of the polypeptide or polymerase of SEQ ID NO:16; residues 1-19, 2-20, 3-21, . . . , 853-871 of the polypeptide or polymerase of SEQ ID NO:17; residues 1-19, 2-20, 3-21, . . . , 853-871 of the polypeptide or polymerase of SEQ ID NO:18; residues 1-19, 2-20, 3-21, . . . , 853-871 of the polypeptide or polymerase of SEQ ID NO:19; residues 1-19, 2-20, 3-21, . . . , 882-900 of the polypeptide or polymerase of SEQ ID NO:20; residues 1-19, 2-20, 3-21, . . . , 846-864 of the polypeptide or polymerase of SEQ ID NO:21; residues 1-19, 2-20, 3-21, . . . , 866-884 of the polypeptide or polymerase of SEQ ID NO:22; residues 1-19, 2-20, 3-21, . . . , 852-870 of the polypeptide or polymerase of SEQ ID NO:23; residues 1-19, 2-20, 3-21, . . . , 910-928 of the polypeptide or polymerase of SEQ ID NO:24; residues 1-19, 2-20, 3-21, . . . , 942-960 of the polypeptide or polymerase of SEQ ID NO:25.

Polypeptide fragments of the invention may be at least 20 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 20 amino acid long fragments including amino acid residues 1-20, 2-21, 3-22, . . . , 901-920 of the polypeptide or polymerase of SEQ ID NO:14; residues 1-20, 2-21, 3-22, . . . , 870-889 of the polypeptide or polymerase of SEQ ID NO:15; residues 1-20, 2-21, 3-22, . . . , 906-925 of the polypeptide or polymerase of SEQ ID NO:16; residues 1-20, 2-21, 3-22, . . . , 852-871 of the polypeptide or polymerase of SEQ ID NO:17; residues 1-20, 2-21, 3-22, . . . , 852-871 of the polypeptide or polymerase of SEQ ID NO:18; residues 1-20, 2-21, 3-22, . . . , 852-871 of the polypeptide or polymerase of SEQ ID NO:19; residues 1-20, 2-21, 3-22, . . . , 881-900 of the polypeptide or polymerase of SEQ ID NO:20; residues 1-20, 2-21, 3-22, . . . , 845-864 of the polypeptide or polymerase of SEQ ID NO:21; residues 1-20, 2-21, 3-22, . . . , 865-884 of the polypeptide or polymerase of SEQ ID NO:22; residues 1-20, 2-21, 3-22, . . . , 851-870 of the polypeptide or polymerase of SEQ ID NO:23; residues 1-20, 2-21, 3-22, . . . , 909-928 of the polypeptide or polymerase of SEQ ID NO:24; residues 1-20, 2-21, 3-22, . . . , 941-960 of the polypeptide or polymerase of SEQ ID NO:25.

Polypeptide fragments of the invention may be at least 21 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 21 amino acid long fragments including amino acid residues 1-21, 2-22, 3-23, . . . , 900-920 of the polypeptide or polymerase of SEQ ID NO:14; residues 1-21, 2-22, 3-23, . . . , 869-889 of the polypeptide or polymerase of SEQ ID NO:15; residues 1-21, 2-22, 3-23, . . . , 905-925 of the polypeptide or polymerase of SEQ ID NO:16; residues 1-21, 2-22, 3-23, . . . , 851-871 of the polypeptide or polymerase of SEQ ID NO:17; residues 1-21, 2-22, 3-23, . . . , 851-871 of the polypeptide or polymerase of SEQ ID NO:18; residues 1-21, 2-22, 3-23, . . . , 851-871 of the polypeptide or polymerase of SEQ ID NO:19; residues 1-21, 2-22, 3-23, . . . , 880-900 of the polypeptide or polymerase of SEQ ID NO:20; residues 1-21, 2-22, 3-23, . . . , 844-864 of the polypeptide or polymerase of SEQ ID NO:21; residues 1-21, 2-22, 3-23, . . . , 864-884 of the polypeptide or polymerase of SEQ ID NO:22; residues 1-21, 2-22, 3-23, . . . , 850-870 of the polypeptide or polymerase of SEQ ID No:23; residues 1-21, 2-22, 3-23, . . . , 908-928 of the polypeptide or polymerase of SEQ ID NO:24; residues 1-21, 2-22, 3-23, . . . , 940-960 of the polypeptide or polymerase of SEQ ID NO:25.

Polypeptide fragments of the invention may be at least 22 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 22 amino acid long fragments including amino acid residues 1-22, 2-23, 3-24, . . . , 899-920 of the polypeptide or polymerase of SEQ ID NO:14; residues 1-22, 2-23, 3-24, . . . , 868-889 of the polypeptide or polymerase of SEQ ID NO:15; residues 1-22, 2-23, 3-24, . . . , 904-925 of the polypeptide or polymerase of SEQ ID NO:16; residues 1-22, 2-23, 3-24, . . . , 850-871 of the polypeptide or polymerase of SEQ ID NO:17; residues 1-22, 2-23, 3-24, . . . , 850-871 of the polypeptide or polymerase of SEQ ID NO:18; residues 1-22, 2-23, 3-24, . . . , 850-871 of the polypeptide or polymerase of SEQ ID NO:19; residues 1-22, 2-23, 3-24, . . . , 879-900 of the polypeptide or polymerase of SEQ ID NO:20; residues 1-22, 2-23, 3-24, . . . , 843-864 of the polypeptide or polymerase of SEQ ID NO:21; residues 1-22, 2-23, 3-24, . . . , 863-884 of the polypeptide or polymerase of SEQ ID NO:22; residues 1-22, 2-23, 3-24, . . . , 849-870 of the polypeptide or polymerase of SEQ ID NO:23; residues 1-22, 2-23, 3-24, . . . , 907-928 of the polypeptide or polymerase of SEQ ID NO:24; residues 1-22, 2-23, 3-24, . . . , 939-960 of the polypeptide or polymerase of SEQ ID NO:25.

Polypeptide fragments of the invention may be at least 23 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 23 amino acid long fragments including amino acid residues 1-23, 2-24, 3-25, . . . , 898-920 of the polypeptide or polymerase of SEQ ID NO:14; residues 1-23, 2-24, 3-25, . . . , 867-889 of the polypeptide or polymerase of SEQ ID NO:15; residues 1-23, 2-24, 3-25, . . . , 903-925 of the polypeptide or polymerase of SEQ ID NO:16; residues 1-23, 2-24, 3-25, . . . , 849-871 of the polypeptide or polymerase of SEQ ID NO:17; residues 1-23, 2-24, 3-25, . . . , 849-871 of the polypeptide or polymerase of SEQ ID NO:18; residues 1-23, 2-24, 3-25, . . . , 849-871 of the polypeptide or polymerase of SEQ ID NO:19; residues 1-23, 2-24, 3-25, . . . , 878-900 of the polypeptide or polymerase of SEQ ID NO:20; residues 1-23, 2-24, 3-25, . . . , 842-864 of the polypeptide or polymerase of SEQ ID NO:21; residues 1-23, 2-24, 3-25, . . . , 862-884 of the polypeptide or polymerase of SEQ ID NO:22; residues 1-23, 2-24, 3-25, . . . , 848-870 of the polypeptide or polymerase of SEQ ID NO:23; residues 1-23, 2-24, 3-25, . . . , 906-928 of the polypeptide or polymerase of SEQ ID NO:24; residues 1-23, 2-24, 3-25, . . . , 938-960 of the polypeptide or polymerase of SEQ ID NO:25.

Polypeptide fragments of the invention may be at least 24 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 24 amino acid long fragments including amino acid residues 1-23, 2-24, 3-25, . . . , 897-920 of the polypeptide or polymerase of SEQ ID NO:14; residues 1-23, 2-24, 3-25, . . . , 866-889 of the polypeptide or polymerase of SEQ ID NO:15; residues 1-23, 2-24, 3-25, . . . , 902-925 of the polypeptide or polymerase of SEQ ID NO:16; residues 1-23, 2-24, 3-25, . . . , 848-871 of the polypeptide or polymerase of SEQ ID NO:17; residues 1-23, 2-24, 3-25, . . . , 848-871 of the polypeptide or polymerase of SEQ ID NO:18; residues 1-23, 2-24, 3-25, . . . , 848-871 of the polypeptide or polymerase of SEQ ID NO:19; residues 1-23, 2-24, 3-25, . . . , 877-900 of the polypeptide or polymerase of SEQ ID NO:20; residues 1-23, 2-24, 3-25, . . . , 841-864 of the polypeptide or polymerase of SEQ ID NO:21; residues 1-23, 2-24, 3-25, . . . , 861-884 of the polypeptide or polymerase of SEQ ID NO:22; residues 1-23, 2-24, 3-25, . . . , 847-870 of the polypeptide or polymerase of SEQ ID NO:23; residues 1-23, 2-24, 3-25, . . . , 905-928 of the polypeptide or polymerase of SEQ ID NO:24; residues 1-23, 2-24, 3-25, . . . , 937-960 of the polypeptide or polymerase of SEQ ID NO:25.

Polypeptide fragments of the invention may be at least 25 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 25 amino acid long fragments including amino acid residues 1-24, 2-25, 3-26, . . . , 896-920 of the polypeptide or polymerase of SEQ ID NO:14; residues 1-24, 2-25, 3-26, . . . , 865-889 of the polypeptide or polymerase of SEQ ID NO:15; residues 1-24, 2-25, 3-26, . . . , 901-925 of the polypeptide or polymerase of SEQ ID NO:16; residues 1-24, 2-25, 3-26, . . . , 847-871 of the polypeptide or polymerase of SEQ ID NO:17; residues 1-24, 2-25, 3-26, . . . , 847-871 of the polypeptide or polymerase of SEQ ID NO:18; residues 1-24, 2-25, 3-26, . . . , 847-871 of the polypeptide or polymerase of SEQ ID NO:19; residues 1-24, 2-25, 3-26, . . . , 876-900 of the polypeptide or polymerase of SEQ ID NO:20; residues 1-24, 2-25, 3-26, . . . , 840-864 of the polypeptide or polymerase of SEQ ID NO:21; residues 1-24, 2-25, 3-26, . . . , 860-884 of the polypeptide or polymerase of SEQ ID NO:22; residues 1-24, 2-25, 3-26, . . . , 846-870 of the polypeptide or polymerase of SEQ ID NO:23; residues 1-24, 2-25, 3-26, . . . , 904-928 of the polypeptide or polymerase of SEQ ID NO:24; residues 1-24, 2-25, 3-26, . . . , 936-960 of the polypeptide or polymerase of SEQ ID NO:25.

Polypeptide fragments of the invention may be at least 26 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 26 amino acid long fragments including amino acid residues 1-25, 2-26, 3-27, . . . , 895-920 of the polypeptide or polymerase of SEQ ID NO:14; residues 1-25, 2-26, 3-27, . . . , 864-889 of the polypeptide or polymerase of SEQ ID NO:15; residues 1-25, 2-26, 3-27, . . . , 900-925 of the polypeptide or polymerase of SEQ ID NO:16; residues 1-25, 2-26, 3-27, . . . , 846-871 of the polypeptide or polymerase of SEQ ID NO:17; residues 1-25, 2-26, 3-27, . . . , 846-871 of the polypeptide or polymerase of SEQ ID NO:18; residues 1-25, 2-26, 3-27, . . . , 846-871 of the polypeptide or polymerase of SEQ ID NO:19; residues 1-25, 2-26, 3-27, . . . , 875-900 of the polypeptide or polymerase of SEQ ID NO:20; residues 1-25, 2-26, 3-27, . . . , 839-864 of the polypeptide or polymerase of SEQ ID NO:21) residues 1-25, 2-26, 3-27, . . . , 859-884 of the polypeptide or polymerase of SEQ ID NO:22; residues 1-25, 2-26, 3-27, . . . , 845-870 of the polypeptide or polymerase of SEQ ID NO:23; residues 1-25, 2-26, 3-27, . . . , 903-928 of the polypeptide or polymerase of SEQ ID NO:24; residues 1-25, 2-26, 3-27, . . . , 935-960 of the polypeptide or polymerase of SEQ ID NO:25.

Polypeptide fragments of the invention may be at least 27 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 27 amino acid long fragments including amino acid residues 1-26, 2-27, 3-28, . . . , 894-920 of the polypeptide or polymerase of SEQ ID NO:14; residues 1-26, 2-27, 3-28, . . . , 863-889 of the polypeptide or polymerase of SEQ ID NO:15; residues 1-26, 2-27, 3-28, . . . , 899-925 of the polypeptide or polymerase of SEQ ID NO:16; residues 1-26, 2-27, 3-28, . . . , 845-871 of the polypeptide or polymerase of SEQ ID NO:17; residues 1-26, 2-27, 3-28, . . . , 845-871 of the polypeptide or polymerase of SEQ ID NO:18; residues 1-26, 2-27, 3-28, . . . , 845-871 of the polypeptide or polymerase of SEQ ID NO:19; residues 1-26, 2-27, 3-28, . . . , 874-900 of the polypeptide or polymerase of SEQ ID NO:20; residues 1-26, 2-27, 3-28, . . . , 838-864 of the polypeptide or polymerase of SEQ ID NO:21; residues 1-26, 2-27, 3-28, . . . , 858-884 of the polypeptide or polymerase of SEQ ID NO:22; residues 1-26, 2-27, 3-28, . . . , 844-870 of the polypeptide or polymerase of SEQ ID NO:23; residues 1-26, 2-27, 3-28, . . . , 902-928 of the polypeptide or polymerase of SEQ ID NO:24; residues 1-26, 2-27, 3-28, . . . , 934-960 of the polypeptide or polymerase of SEQ ID NO:25.

Polypeptide fragments of the invention may be at least 28 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 28 amino acid long fragments including amino acid residues 1-27, 2-28, 3-29, . . . , 893-920 of the polypeptide or polymerase of SEQ ID NO:14; residues 1-27, 2-28, 3-29, . . . , 862-889 of the polypeptide or polymerase of SEQ ID NO:15; residues 1-27, 2-28, 3-29, . . . , 898-925 of the polypeptide or polymerase of SEQ ID NO:16; residues 1-27, 2-28, 3-29, . . . , 844-871 of the polypeptide or polymerase of SEQ ID NO:17; residues 1-27, 2-28, 3-29, . . . , 844-871 of the polypeptide or polymerase of SEQ ID NO:18; residues 1-27, 2-28, 3-29, . . . , 844-871 of the polypeptide or polymerase of SEQ ID NO:19; residues 1-27, 2-28, 3-29, . . . , 873-900 of the polypeptide or polymerase of SEQ ID NO:20; residues 1-27, 2-28, 3-29, . . . , 837-864 of the polypeptide or polymerase of SEQ ID NO:21; residues 1-27, 2-28, 3-29, . . . , 857-884 of the polypeptide or polymerase of SEQ ID NO:22; residues 1-27, 2-28, 3-29, . . . , 843-870 of the polypeptide or polymerase of SEQ ID NO:23; residues 1-27, 2-28, 3-29, . . . , 901-928 of the polypeptide or polymerase of SEQ ID NO:24; residues 1-27, 2-28, 3-29, . . . , 933-960 of the polypeptide or polymerase of SEQ ID NO:25.

Polypeptide fragments of the invention may be at least 29 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 29 amino acid long fragments including amino acid residues 1-28, 2-29, 3-30, . . . , 892-920 of the polypeptide or polymerase of SEQ ID NO:14; residues 1-28, 2-29, 3-30, . . . , 861-889 of the polypeptide or polymerase of SEQ ID NO:15; residues 1-28, 2-29, 3-30, . . . , 897-925 of the polypeptide or polymerase of SEQ ID NO:16; residues 1-28, 2-29, 3-30, . . . , 843-871 of the polypeptide or polymerase of SEQ ID NO:17; residues 1-28, 2-29, 3-30, . . . , 843-871 of the polypeptide or polymerase of SEQ ID NO:18; residues 1-28, 2-29, 3-30, . . . , 843-871 of the polypeptide or polymerase of SEQ ID NO:19; residues 1-28, 2-29, 3-30, . . . , 872-900 of the polypeptide or polymerase of SEQ ID NO:20; residues 1-28, 2-29, 3-30, . . . , 836-864 of the polypeptide or polymerase of SEQ ID NO:21; residues 1-28, 2-29, 3-30, . . . , 856-884 of the polypeptide or polymerase of SEQ ID NO:22; residues 1-28, 2-29, 3-30, . . . , 842-870 of the polypeptide or polymerase of SEQ ID NO:23; residues 1-28, 2-29, 3-30, . . . , 900-928 of the polypeptide or polymerase of SEQ ID NO:24; residues 1-28, 2-29, 3-30, . . . , 932-960 of the polypeptide or polymerase of SEQ ID NO:25.

Polypeptide fragments of the invention may be at least 30 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 30 amino acid long fragments including amino acid residues 1-29, 2-30, 3-31, . . . , 891-920 of the polypeptide or polymerase of SEQ ID NO:14; residues 1-29, 2-30, 3-31, . . . , 860-889 of the polypeptide or polymerase of SEQ ID NO:15; residues 1-29, 2-30, 3-31, . . . , 896-925 of the polypeptide or polymerase of SEQ ID NO:16; residues 1-29, 2-30, 3-31, . . . , 842-871 of the polypeptide or polymerase of SEQ ID NO:17; residues 1-29, 2-30, 3-31, . . . , 842-871 of the polypeptide or polymerase of SEQ ID NO:18; residues 1-29, 2-30, 3-31, . . . , 842-871 of the polypeptide or polymerase of SEQ ID NO:19; residues 1-29, 2-30, 3-31, . . . , 871-900 of the polypeptide or polymerase of SEQ ID NO:20; residues 1-29, 2-30, 3-31, . . . , 835-864 of the polypeptide or polymerase of SEQ ID NO:21; residues 1-29, 2-30, 3-31, . . . , 855-884 of the polypeptide or polymerase of SEQ ID NO:22 residues 1-29, 2-30, 3-31, . . . , 841-870 of the polypeptide or polymerase of SEQ ID NO:23; residues 1-29, 2-30, 3-31, . . . , 899-928 of the polypeptide or polymerase of SEQ ID NO:24; residues 1-29, 2-30, 3-31, . . . , 931-960 of the polypeptide or polymerase of SEQ ID NO:25.

Polypeptide fragments of the invention may be at least 31 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 31 amino acid long fragments including amino acid residues 1-30, 2-31, 3-32, . . . , 890-920 of the polypeptide or polymerase of SEQ ID NO:14; residues 1-30, 2-31, 3-32, . . . , 859-889 of the polypeptide or polymerase of SEQ ID NO:15; residues 1-30, 2-31, 3-32, . . . , 895-925 of the polypeptide or polymerase of SEQ ID NO:16; residues 1-30, 2-31, 3-32, . . . , 841-871 of the polypeptide or polymerase of SEQ ID NO:17; residues 1-30, 2-31, 3-32, . . . , 841-871 of the polypeptide or polymerase of SEQ ID NO:18; residues 1-30, 2-31, 3-32, . . . , 841-871 of the polypeptide or polymerase of SEQ ID NO:19; residues 1-30, 2-31, 3-32, . . . , 870-900 of the polypeptide or polymerase of SEQ ID NO:20; residues 1-30, 2-31, 3-32, . . . , 834-864 of the polypeptide or polymerase of SEQ ID NO:21; residues 1-30, 2-31, 3-32, . . . , 854-884 of the polypeptide or polymerase of SEQ ID NO:22; residues 1-30, 2-31, 3-32, . . . , 840-870 of the polypeptide or polymerase of SEQ ID NO:23; residues 1-30, 2-31, 3-32, . . . , 898-928 of the polypeptide or polymerase of SEQ ID NO:24; residues 1-30, 2-31, 3-32, . . . , 930-960 of the polypeptide or polymerase of SEQ ID NO:25.

Polypeptide fragments of the invention may be at least 32 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 32 amino acid long fragments including amino acid residues 1-31, 2-32, 3-33, . . . , 889-920 of the polypeptide or polymerase of SEQ ID NO:14; residues 1-31, 2-32, 3-33, . . . , 858-889 of the polypeptide or polymerase of SEQ ID NO:15; residues 1-31, 2-32, 3-33, . . . , 894-925 of the polypeptide or polymerase of SEQ ID NO:16; residues 1-31, 2-32, 3-33, . . . , 840-871 of the polypeptide or polymerase of SEQ ID NO:17; residues 1-31, 2-32, 3-33, . . . , 840-871 of the polypeptide or polymerase of SEQ ID NO:18; residues 1-31, 2-32, 3-33, . . . , 840-871 of the polypeptide or polymerase of SEQ ID NO:19; residues 1-31, 2-32, 3-33, . . . , 869-900 of the polypeptide or polymerase of SEQ ID NO:20; residues 1-31, 2-32, 3-33, . . . , 833-864 of the polypeptide or polymerase of SEQ ID NO:21; residues 1-31, 2-32, 3-33, . . . , 853-884 of the polypeptide or polymerase of SEQ ID NO:22; residues 1-31, 2-32, 3-33, . . . , 839-870 of the polypeptide or polymerase of SEQ ID NO:23; residues 1-31, 2-32, 3-33, . . . , 897-928 of the polypeptide or polymerase of SEQ ID NO:24; residues 1-31, 2-32, 3-33, . . . , 929-960 of the polypeptide or polymerase of SEQ ID NO:25.

Polypeptide fragments of the invention may be at least 33 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 33 amino acid long fragments including amino acid residues 1-32, 2-33, 3-34, . . . , 888-920 of the polypeptide or polymerase of SEQ ID NO:14; residues 1-32, 2-33, 3-34, . . . , 857-889 of the polypeptide or polymerase of SEQ ID NO:15; residues 1-32, 2-33, 3-34, . . . , 893-925 of the polypeptide or polymerase of SEQ ID NO:16; residues 1-32, 2-33, 3-34, . . . , 839-871 of the polypeptide or polymerase of SEQ ID NO:17; residues 1-32, 2-33, 3-34, . . . , 839-871 of the polypeptide or polymerase of SEQ ID NO:18; residues 1-32, 2-33, 3-34, . . . , 839-871 of the polypeptide or polymerase of SEQ ID NO:19; residues 1-32, 2-33, 3-34, . . . , 868-900 of the polypeptide or polymerase of SEQ ID NO:20; residues 1-32, 2-33, 3-34, . . . , 832-864 of the polypeptide or polymerase of SEQ ID NO:21; residues 1-32, 2-33, 3-34, . . . , 852-884 of the polypeptide or polymerase of SEQ ID NO:22; residues 1-32, 2-33, 3-34, . . . , 838-870 of the polypeptide or polymerase of SEQ ID NO:23; residues 1-32, 2-33, 3-34, . . . , 896-928 of the polypeptide or polymerase of SEQ ID NO:24; residues 1-32, 2-33, 3-34, . . . , 928-960 of the polypeptide or polymerase of SEQ ID NO:25.

Polypeptide fragments of the invention may be at least 34 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, polypeptides of the invention may comprise or consist of 34 amino acid long fragments including amino acid residues 1-33, 2-34, 3-35, . . . , 887-920 of the polypeptide or polymerase of SEQ ID NO:14; residues 1-33, 2-34, 3-35, . . . , 856-889 of the polypeptide or polymerase of SEQ ID NO:15; residues 1-33, 2-34, 3-35, . . . , 892-925 of the polypeptide or polymerase of SEQ ID NO:16; residues 1-33, 2-34, 3-35, . . . , 838-871 of the polypeptide or polymerase of SEQ ID NO:17; residues 1-33, 2-34, 3-35, . . . , 838-871 of the polypeptide or polymerase of SEQ ID NO:18; residues 1-33, 2-34, 3-35, . . . , 838-871 of the polypeptide or polymerase of SEQ ID NO:19; residues 1-33, 2-34, 3-35, . . . , 867-900 of the polypeptide or polymerase of SEQ ID NO:20; residues 1-33, 2-34, 3-35, . . . , 831-864 of the polypeptide or polymerase of SEQ ID NO:21; residues 1-33, 2-34, 3-35, . . . , 851-884 of the polypeptide or polymerase of SEQ ID NO:22; residues 1-33, 2-34, 3-35, . . . , 837-870 of the polypeptide or polymerase of SEQ ID NO:23; residues 1-33, 2-34, 3-35, . . . , 895-928 of the polypeptide or polymerase of SEQ ID NO:24; residues 1-33, 2-34, 3-35, . . . , 927-960 of the polypeptide or polymerase of SEQ ID NO:25.

An antibody of the invention may specifically bind one of the above fragments, or more than one fragments which overlap. Thus, the invention also includes antibodies which bind one or more polypeptides of the invention as well as methods for making such antibodies and compositions comprising such antibodies.

Polypeptide fragments of the invention may contain a continuous series of deleted residues from the amino (N)- or the carboxyl (C)-terminus, or both. For example, any number of amino acids, ranging from 1 to 981, can be deleted from the N-terminus. Polypeptides of the invention may comprise or consist of fragments containing a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, 310 to 320, 320 to 330, 330 to 340, 340 to 350, 350 to 360, 360 to 370, 370 to 380, 380 to 390, 390 to 400, 400 to 410, 410 to 420, 420 to 430, 430 to 440, 440 to 450, 450 to 460, 460 to 470, or 470 to 480 amino acids from the N-terminus of the full length polypeptide or the full length polymerase (e.g., the polypeptides of SEQ ID NOS:14-25 with or without the N-terminal amino acids encoded by the vectors, or the polymerases encoded by the deposited clones).

As another example, any number of amino acids, ranging from 1 to 981, can be deleted from the C-terminus. Polypeptides of the invention may comprise or consist of fragments containing a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, 310 to 320, 320 to 330, 330 to 340, 340 to 350, 350 to 360, 360 to 370, 370 to 380, 380 to 390, 390 to 400, 400 to 410, 410 to 420, 420 to 430, 430 to 440, 440 to 450, 450 to 460, 460 to 470, or 470 to 480 amino acids from the C-terminus of the full length polypeptide or the full length polymerase (e.g., the polypeptides of SEQ ID NOS:14-25 with or without the N-terminal amino acids encoded by the vectors, or the polymerases encoded by the deposited clones).

Furthermore, polypeptides of the invention may comprise or consist of fragments which contain combinations of N- and C-terminal deletions such as the N-terminal and C-terminal deletions deletions described above. Combined N- and C-terminal deletion fragments of the invention may contain a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, 310 to 320, 320 to 330, 330 to 340, 340 to 350, 350 to 360, 360 to 370, 370 to 380, 380 to 390, 390 to 400, 400 to 410, 410 to 420, 420 to 430, 430 to 440, 440 to 450, 450 to 460, 460 to 470, or 470 to 480 amino acids from the N-terminus and may also contain a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, 310 to 320, 320 to 330, 330 to 340, 340 to 350, 350 to 360, 360 to 370, 370 to 380, 380 to 390, 390 to 400, 400 to 410, 410 to 420, 420 to 430, 430 to 440, 440 to 450, 450 to 460, 460 to 470, or 470 to 480 amino acids from the C-terminus.

Thus, exemplary polypeptides of the invention include polypeptides which comprise or consist of amino acids 33 to 840, 56 to 851, 73 to 893, 11 to 235, 450 to 863, 578 to 901, 435 to 920, 31 to 121, 41 to 93, 235 to 298, 425 to 779, or 534 to 859 of the full length polypeptide or the polymerase of SEQ ID NO:14. Additional exemplary of polypeptides of the invention include polypeptides which comprise or consist of amino acids 55 to 810, 67 to 878, 73 to 803, 11 to 240, 461 to 877, 578 to 889, 435 to 888, 41 to 142, 41 to 93, 235 to 303, 425 to 765, or 523 to 855 of the full length polypeptide or the polymerase in SEQ ID NO: 15. Other exemplary of polypeptides of the invention include polypeptides which comprise or consist of amino acids 55 to 810, 67 to 844, 73 to 779, 11 to 253, 461 to 852, 578 to 787, 435 to 831, 41 to 122, 48 to 93, 225 to 303, 455 to 765, or 513 to 845 of the full length polypeptide or the polymerase in SEQ ID NOS:16-25. The invention further includes nucleic acid molecules which encodes these polypeptides of the invention, as well as other polypeptides described herein, and host cells which contain such nucleic acid molecules. The invention further includes methods for making polypeptides of the invention (e.g., methods for producing polypeptides using nucleic acid molecules of the invention). In particular embodiments, polypeptides of the invention are provided in (1) isolated, (2) substantially pure, and/or (3) essentially pure forms. The invention further includes compositions and mixtures (e.g., reaction mixtures) which contain one or more polypeptides and/or polynucleotides of the invention.

Even if deletion of one or more amino acids from the N- and/or C-terminus of a protein results in modification of loss of one or more biological functions of the protein, other functional activities (e.g., enzymatic activities, antigenic activity, immunogenic activity) may still be retained. For example, the ability of shortened polypeptides to induce and/or bind to antibodies which recognize the complete forms of the polypeptides generally will be retained when less than the majority of the residues of the complete or mature polypeptide are removed from the N- and/or C-terminus. Whether a particular polypeptide lacking N- and/or C-terminal residues of a complete polypeptide retains such immunologic activities can readily be determined by routine methods described herein and otherwise known in the art. It is not unlikely that a fragment with a large number of deleted N- and/or C-terminal amino acid residues may retain some antigenic or immunogenic activities. In fact, peptides composed of as few as six amino acid residues may often evoke an immune response, as discussed below.

Polypeptide fragments of the invention may include unique regions, i.e., stretches of amino acids of the polymerases of SEQ ID NOS:14-25 that are less than 100% identical to corresponding stretches of amino acids in other proteins such the polypeptides of SEQ ID NOS:27-34). Unique regions of each polypeptide (e.g., polymerase) of the invention are shown in the alignment in Table 35, which indicates the identical and non-identical amino acids of the polymerases of SEQ ID NOS:14-25 (or the polymerases encoded by a deposited clone) as compared to the polypeptides of SEQ ID NOS:27-34. Polypeptide fragments of the invention containing unique regions are useful for generating highly specific antibodies of the invention, as discussed below, and for conferring upon a protein a particular activity, such as an enzymatic activity described herein. Thus, fragments containing unique regions are preferred antigenic fragments of the invention. Additionally, fragments containing unique regions are also useful for producing fusion proteins such as proteins produced by DNA shuffling, described in more detail below. Using DNA shuffling, fusion proteins are constructed which comprise fragments from one or more polymerases and which preferably have an enzymatic activity of a polymerase of SEQ ID NOS:14-25 or the polymerases encoded by a deposited clone.

Other fragments of the invention are fragments characterized by structural or functional attributes of the polypeptides of the invention. Such fragments include amino acid residues that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson-Wolf program) of polypeptides of the invention (e.g., the polypeptides or polymerases of SEQ ID NOS:14-25). Certain preferred regions include, but are not limited to, regions of the aforementioned types identified by analysis of the amino acid sequence depicted in SEQ ID NOS:14-25, such preferred regions include; Garnier-Robson predicted alpha-regions, beta-regions, turn-regions, and coil-regions; Chou-Fasman predicted alpha-regions, beta-regions, turn-regions, and coil-regions; Kyte-Doolittle predicted hydrophilic and hydrophobic regions; Eisenberg alpha and beta amphipathic regions; Emini surface-forming regions; and Jameson-Wolf high antigenic index regions, as predicted using the default parameters of these computer programs. These structural or functional attributes can be generated using the various modules and algorithms of the DNA*STAR program set on default parameters.

Among preferred polypeptide fragments of the invention in this regard are those that comprise regions of the polypeptides that combine several structural features, such as several of the features set out above or below.

In another embodiment, the polypeptide may comprise or consist of one or more polypeptide fragments (e.g., regions) such as a polypeptide fragment of the invention described herein. For a polypeptide comprising or consisting of the amino acid sequence of two or more fragments (e.g., regions), the fragments (e.g., regions) may be contiguous with one another. In one embodiment, the fragments (e.g., regions) are not contiguous with one another, i.e., they are separated by one or more amino acid residues.

Preferably, the fragments (e.g., regions) align with the corresponding regions of the full length polypeptide such that they are separated by the same number of amino acid residues as separate them in the full length polypeptide or the full length polymerase (e.g., the polypeptides of SEQ ID NOS:14-25 with or without the N-terminal amino acids encoded by the vectors, (or the polymerases encoded by the deposited clones), or alternatively, the polypeptides of SEQ ID NOS:27-34.

Polypeptide fragments of the invention may contain antigenic regions (i.e., regions to which an antibody will bind; epitopes) of the polypeptides of the invention. Antigenic regions may be as small as 6 amino acids.

Polypeptide fragments of the invention which function as antigenic epitopes may be produced by any conventional means. See, e.g., Houghten, R. A., Proc. Natl. Acad. Sci. USA 82:5131-5135 (1985) further described in U.S. Pat. No. 4,631,211.

As to the selection of fragments bearing an antigenic region, it is well known in that art that relatively short synthetic peptides that mimic part of a protein sequence are routinely capable of eliciting an antiserum that reacts with the partially mimicked protein. See, e.g., Sutcliffe, J. G., Shinnick, T. M., Green, N. and Learner, R. A., Science 219:660-666 (1983).

Polypeptide fragments of the invention capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of fewer than six residues generally are ineffective at inducing antibodies that bind to the mimicked protein; longer, peptides, especially those containing proline residues, usually are effective. Sutcliffe et al., supra, at 661. For instance, 18 of 20 peptides designed according to these guidelines, containing 8-39 residues covering 75% of the sequence of the influenza virus hemagglutinin HA1 polypeptide chain, induced antibodies that reacted with the HA1 protein or intact virus; and 12/12 peptides from the MuLV polymerase and 18/18 from the rabies glycoprotein induced antibodies that precipitated the respective proteins. Thus, the invention includes polypeptides comprising or consisting of fragments of the full length polypeptide or the full length polymerase (e.g., the polypeptides of SEQ ID NOS:14-25 with or without the N-terminal amino acids encoded by the vectors, (or the polymerases encoded by the deposited clones) which are at least 6, 10, 12, 14, 18, or 20 amino acids in length and have one or more of the following features: (1) is not extremely hydrophobic, and/or (2) contains one or more proline residues.

Antigenic fragments of the invention, and polypeptides comprising them, are therefore useful to raise antibodies, including monoclonal antibodies, that bind specifically to a polypeptide of the invention. Thus, a high proportion of hybridomas obtained by fusion of spleen cells from donors immunized with an antigen epitope-bearing peptide generally secrete antibody that binds the native protein. Sutcliffe et al., supra, at 663. The antibodies raised by antigenic fragments or polypeptides comprising them are useful to detect the polypeptides of the invention, and antibodies to different fragments may be used for tracking the fate of various regions of a protein precursor which undergoes post-translational processing. The fragments and anti-fragment antibodies may be used in a variety of qualitative or quantitative assays for the mimicked protein, for instance in competition assays since it has been shown that even short peptides (e.g. about 9 amino acids) can bind and displace the larger peptides in immunoprecipitation assays. See, for instance, Wilson et al., Cell 37:767-778 (1984) at 777. The antibodies of the invention also are useful for purification of the polypeptides of the invention, for instance, by adsorption chromatography using methods well known in the art.

Antigenic fragments and polypeptides of the invention designed according to the above guidelines preferably contain a sequence of at least seven, more preferably at least nine and most preferably between about 15 to about 30 amino acids contained within the amino acid sequence of a polypeptide of the invention. However, fragments and polypeptides comprising, or alternatively consisting of, a larger portion such as about 30 to about 50 amino acids, or any length up to and including the entire amino acid sequence of a polypeptide of the invention, also are considered antigenic fragments or polypeptides of the invention and also are useful for inducing antibodies that react with the full length polypeptide. Preferably, the amino acid sequence of the antigenic fragment is selected to provide substantial solubility in aqueous solvents (i.e., the sequence includes relatively hydrophilic residues and highly hydrophobic sequences are preferably avoided); and sequences containing proline residues are particularly preferred.

In the present invention, antigenic fragments preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred polypeptides comprising antigenic fragments are at least 10, 15, 20, 25, 30, 35, 40, 45, 50, 55, 60, 65, 70, 75, 80, 85, 90, 95, or 100 amino acid residues in length. Additional non-exclusive preferred antigenic fragments include the fragments disclosed herein, as well as portions thereof. Antigenic fragments are useful, for example, to raise antibodies, including monoclonal antibodies, that specifically bind the epitope. Preferred antigenic fragments include the fragments disclosed herein, as well as any combination of two, three, four, five or more of these fragments. Antigenic fragments can be used as the target molecules in immunoassays. (See, for instance, Wilson et al., Cell 37:767-778 (1984); Sutcliffe et al., Science 219:660-666 (1983)).

Similarly, antigenic fragments can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). The polypeptides comprising, or alternatively consisting of, one or more antigenic fragments may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, antigenic fragments comprising as few as 8 to 10 amino acids have been shown to be sufficient to raise antibodies capable of binding to, at the very least, linear epitopes in a denatured polypeptide (e.g., in Western blotting).

Polypeptides of the invention may comprise or consist of variants of the full length polypeptide or the full length polymerase (e.g., the polypeptides of SEQ ID NOS:14-25 with or without the N-terminal amino acids encoded by the vectors), variants of the polypeptides encoded by the deposited clones, and variants of the fragments described above. Variants include polypeptides which are at least 80%, 81%, 82%, 83%, 84%, 85%, 86%, 87%, 88%, 89%, 90%, 91%, 92%, 93%, 94%, 95% 96%, 97%, 98%, or 99% identical to a polypeptide encoded by a deposited clone, to a polypeptide or polymerase of SEQ ID NOS:14-25, or to a fragment described above.

Thus, the invention includes, in part, polypeptides which are at least 80%, 81%, 82%, 83%, 84%, 85%, 86%, 87%, 88%, 89%, 90%, 91%, 92%, 93%, 94%, 95% 96%, 97%, 98%, or 99% identical to (1) a polypeptide encoded by a deposited clone described herein, (2) to a polypeptide or polymerase having an amino acid sequence set out in SEQ ID NOS:14-25, or (3) to a subportion of one of these polypeptides or polymerases (e.g., amino acids 125-333, 156-392, or 450-771 of a polypeptide or polymerase having an amino acid sequence set out in SEQ ID NO:14). The invention further includes nucleic acid molecules which encode these polypeptides, as well as host cells which contain such nucleic acid molecules. The invention also includes compositions and mixtures (e.g., reaction mixtures) which contain one or more polypeptides and/or polynucleotides of the invention.

In many instances, the above described polypeptides, as well as other polypeptides of the invention, will have one or more activity associated with a polypeptide encoded by a deposited clone described herein or a polypeptide or polymerase having an amino acid sequence set out in SEQ ID NOS:14-25.

It will be recognized in the art that some amino acid sequences of the polypeptides of the invention can be varied without significant affect on the structure or function of the protein. If such differences in sequence are contemplated, it should be remembered that there may be critical areas on the protein which determine activity. In general, it is possible to replace residues which form the tertiary structure, provided that residues performing a similar structural or enzymatic function are used. In other instances, the type of residue may be completely unimportant if the alteration occurs at a non-critical region of the protein.

Thus, the invention includes variants which may show a functional activity. Preferably, the variants demonstrate a functional activity such as antigenicity or an enzymatic activity described above (e.g., a DNA polymerase activity such as DNA-dependent DNA polymerase activity and/or reverse transriptase activity).

The functional activity of polypeptides of the invention can be assayed by various methods. For example, in one embodiment where one is assaying for antigenicity, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In addition, assays described herein and otherwise known in the art may routinely be applied to measure the ability of variants to elicit an enzymatic activity.

Variants include deletions, insertions, inversions, repeats, and substitutions (e.g., conservative substitutions, non-conservative substitutions, type substitutions (for example, substituting one hydrophilic residue for another hydrophilic residue, but not a strongly hydrophilic for a strongly hydrophobic, as a rule), primary shifts, primary transpositions, secondary transpositions, and coordinated replacements).

More than one amino acid (e.g., 2, 3, 4, 5, 6, 7, 8, 9, 10, etc.) can be deleted or inserted or can be substituted with another amino acid as described above (either conservative or nonconservative). The deletion, insertion, or substitution can occur in the full length, mature, or proprotein form of the polypeptide, as well as in the fragments described above.

Variants may contain at least one amino acid substitution, deletion or insertion but not more than 50 (e.g., 15, 18, 20, 30, 35, 40, etc.) amino acid substitutions, deletions or insertions, even more preferably, not more than 40 amino acid substitutions, deletions or insertions, still more preferably, not more than 30 amino acid substitutions, deletions or insertions, and still even more preferably, not more than 20 amino acid substitutions, deletions or insertions. Of course, in order of increasing preference, it is preferable for a variant to contain at least one, but not more than 10, 9, 8, 7, 6, 5, 4, 3, 2 or 1 amino acid substitutions, deletions or insertions. In specific embodiments, the number of additions, substitutions, and/or deletions in the polypeptide (e.g., the full length form and/or fragments described herein), is 1-5, 5-10, 5-25, 5-50, 10-50 or 50-150. Conservative amino acid substitutions are preferable in some embodiments.

Of course, the number of amino acid substitutions a skilled artisan would make depends on many factors, including those described above and below.

Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. (See Table 41).

Of additional special interest are also substitutions of charged amino acids with another charged amino acid or with neutral amino acids. This may result in proteins with improved characteristics such as less aggregation. Prevention of aggregation is highly desirable. Aggregation of proteins can result in a reduced activity.

Guidance concerning how to make phenotypically silent amino acid substitutions is provided in Bowie, J. U. et al., wherein the authors indicate that there are two main strategies for studying the tolerance of an amino acid sequence to change. Bowie, J. U. et al., “Deciphering the Message in Protein Sequences: Tolerance to Amino Acid Substitutions,” Science 247:1306-1310 (1990)

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for functional activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved.

Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Thus, residues important for a particular functional activity (e.g., enzymatic, antigenic or immunogenic activity) may be identified by mutagenesis strategies designed to locally perturb the protein. In alanine scanning mutagenesis, all non-alanine residues of the protein (or of a region of the protein suspected to contain the binding site are replaced, one-by-one, with alanine, yielding a collection of single substitution mutants. Alanine is used because (1) it is the most common amino acid residue in proteins, (2) it has a small side chain, and therefore is not likely to sterically hinder other residues, and (3) its side chain does not form H-bonds, but is not especially hydrophobic. Cunningham and Wells (1989) conducted an Ala scanning mutagenesis study of residues 2-19, 54-74, and 167-191 in hGH. A total of 62 Ala mutations were produced. Of these, fourteen mutants destabilized the protein, eleven mutants seemingly enhanced activity. Of the remaining 37 mutants, only four impaired binding by 10-fold or more, and only nine by 5-fold or more. See generally WO90/04788.

For other uses of Ala-scan mutagenesis, see Yu et al (1995) (complete scan of a single disulfide derivative of the 58-residue protein BPTI); Allen et al (1987) (Ala-scan of residues 52-61 of hen egg white lysozyme); Ruf et al (1994) (Ala-scan of residues other than Gly, Pro and Cys; multiple Ala mutants examined first, then single Ala mutants); Williams et al (1995) (Ala-scan in insulin receptor of (1) charged amino acids, (2) aromatic residues, and (3) residues adjacent to (1) or (2), other than prolines, cysteines, or potential N-linked glycosylation sites); Kelly et al (1993) (Ala-scan of antibody CDR). Ala-scanning mutagenesis may be applied to all residues of a protein, or to residues selected on some rational basis, such as amino acid type (e.g., charged and aromatic residues), degree of variability in a homologous protein family, or relevance to function as shown by homologue-scanning mutagenesis.

Preferably, further mutations (especially non-conservative mutations) are made at sites where an alanine substitution does not lead to a decrease in an activity of interest of more than 20-fold, more preferably, of more than 10-fold, even more preferably, of more than 5-fold, still more preferably, of more than 2-fold. Most preferably, mutations are made at sites at which an alanine substitution improves activity.

Preferably, when multiple mutations are made, the expected (additive) effect of the mutations is one which does not lead to a decrease in activity of more than 10-fold, more preferably, of more than 5 fold, still more preferably, of more than two fold. Most preferably, the expected effect is to improve activity. The expected effect of a conservative substitution is the effect of that mutation as a single substitution if known, or otherwise neutral. The expected effect of a non-conservative substitution is the effect of that mutation as a single substitution if known, or otherwise the effect of a single substitution of a different residue of the same exchange group as the actual replacement residue, if known, or otherwise the effect of a single Ala substitution.

Another approach is homologue-scanning mutagenesis. This involves identifying a homologue which can be distinguished in an activity assay from the protein of interest, and screening mutants in which a segment of the protein of interest is replaced by corresponding segments of the homologue (or vice versa). Proteins that may be used as homologues include previously identified polymerases such as those encoded in SEQ ID NOS 27-34 or otherwise known in the art. If the replacement alters the activity of the modified protein, the segment in question presumably contributes to the observed difference in activity between the protein of interest and the homologous protein, and comparison of the interchanged segments helps to explain the character of the binding site involved in that activity. For example, segments of prolactin, which does not bind the GH receptor, have been used to replace segments of growth hormone, which does. If a substitution disrupts GH binding, it implies that the replaced segment was part of the GH receptor binding site, and one may then focus on how the replaced and replacing segments differ. (See WO90/04788).

If a residue is determined to be a part of the enzymatic or binding site, one may prepare all possible single substitution mutants of that site.

It is possible to incorporate two or more tolerable mutations into a protein. Generally speaking, as a first approximation, it is reasonable to assume that the effect of two or more mutations will be additive in nature.

Non-additive effects are more likely to occur between residues that are in Van der Waals contact with each other. See Sandberg and Terwilliger (1993). According to Schreiber and Fersht (1995), non-additive effects are more likely to occur between residues less than 7 Angstroms apart (10 Angstroms in the case of charged residues). The effect of a second mutation on a first one may be synergistic, additive, partially additive, neutral, antagonistic, or suppressive. Long range but low magnitude departures from additivity may occur reasonably often, see LiCata and Ackers (1995), but do not significantly impair the value of multiple mutation in protein engineering.

Gregoret et al (1993) assumed that, under selective conditions, the frequency of occurrence of a mutation in an active mutant was an indication of whether the mutant conferred resistance, and found that an additive model (multiplying the mutational frequencies of a pair of single Ala substitution mutants) was about 90% effective in predicting the activity class of a binomial (multiple Ala substitution) mutant.

The most common reason for combining mutations is to benefit from their additive or synergistic effect in combination. For example, if a mutation has both favorable and unfavorable activities, it may be possible to combine it with a second mutation that neutralizes the unfavorable activity of the first mutation.

One use of multiple mutations is to achieve, by combining mutations which individually have a small but favorable effect on activity, a mutant with a more substantial improvement in activity. It is not necessary that the mutations be strictly additive; it is sufficient that they be at least partially additive for the combination to be advantageous.

The interactivity of two residues is generally determined by preparing both single substitution mutants as well as a double substitution mutant, and determining whether the effects are additive or not. Therefore, if single Ala substitutions have been shown to favorably or unfavorably affect activity, one may prepare a double Ala mutant and compare its activity to that of the single substitution mutants. While it is certainly possible that two mutations which, by themselves, do not affect activity, may do so when combined, this is unlikely, especially if the sites are not close together.

One could prepare all possible double Ala mutants, which would mean preparing N(N-1) mutants, where N was the number of non-Ala residues in the protein. In general, it is preferable to limit the double substitution studies to sites known to favorably affect the activity. Possibly, one would also consider sites which were strongly unfavorable (to look for antagonistic interactions).

Another approach is binomial Ala-scanning mutagenesis. Here, one constructs a library in which, at each position of interest of a given protein molecule, the residue is randomly either the native residue, or Ala. See Gregoret and Sauer (1993). It is feasible to screen a library of 10¹⁰ mutants, so the combined effects of up to 30 different Ala substitutions (about 2²⁷ to about 10¹⁰) can be studied in one experiment. It should be noted that the Ala:non-Ala ratio at each position may be, but need not be equal.

If the protein is too large for all sites of interest to be sampled by binomial Ala-scanning mutagenesis in a single experiment, one may divide the protein into segments and subject each segment in turn to such mutagenesis, and then, as a cross-check, similarly mutate one residue from each segment.

Even when mutations are not additive in effect, this is may be desirable. Green and Shortle, (1993) reported that mutations which individually reduced stability, when not additive in their effects, were almost exclusively sub-additive, i.e., the reduction in stability was less than that expected by summing the individual destabilizations. This is credited to an overlap of the “spheres of perturbation” surrounding the two mutations. Ballinger et al (1995) reported that a combination subtilisin BPN′ mutant had a larger than additive shift in specificity toward dibasic substrates, which is a desirable change.

Certain multiple mutations are worthy of special comment, as follows.

Primary shifts: In a primary shift the residue at position n becomes the replacement amino acid at position n+s, or vice versa. For example, instead of Cys at 30, one might have Cys at 31. The result is a mere displacement, rather than a loss, of the amino acid in question. In a primary shift, s (the shift distance) is most often equal to one, but may be two, three or more. The greater the value of s, the more the shift resembles an ordinary double mutation.

Primary transpositions: In a primary transposition, the residues at positions n and n+s in the primary amino acid sequence are swapped. Such swaps are less likely to perturb the protein than the individual replacements, examined singly, might suggests. A primary transposition is, in effect, a combination of two complementary shifts.

Secondary Transposition: Here, two amino acids which interact as a result of the folding of the protein are swapped. A classic example would be members of a salt bridge. If there is an Asp in one segment forming a salt bridge with a Lys in another segment, the Asp and Lys can be swapped, and a salt bridge can still form.

Coordinated Replacement: Here, replacement of residue x is coordinated with replacement of residue y. Thus, replacement of one Cys may be coordinated with replacement of a second Cys with which it otherwise forms a disulfide bond, and if one amino acid of a pair forming a salt bridge is replaced by an uncharged a.a., the other may likewise be replaced.

Primary shifts, primary transpositions, secondary transpositions and coordinated replacements are more likely to be tolerated than other multiple mutations involving the same individual amino acid changes.

Examples of production of amino acid substitutions in proteins which can be used for obtaining variants of the present invention include any known method steps, such as presented in U.S. Pat. No. RE 33,653, U.S. Pat. Nos. 4,959,314, 4,588,585 and 4,737,462, to Mark et al; U.S. Pat. No. 5,116,943 to Koths et al, U.S. Pat. No. 4,965,195 to Namen et al; U.S. Pat. No. 4,879,111 to Chong et al; and U.S. Pat. No. 5,017,691 to Lee et al; and lysine substituted proteins presented in U.S. Pat. No. 4,904,584 (Shaw et al).

Polypeptides of the invention may be altered by being subjected to random mutagenesis by error-prone PCR, random nucleotide insertion or other methods prior to recombination. Polypeptides of the invention may be produced by DNA shuffling, gene-shuffling, motif-shuffling, exon-shuffling, and/or codon-shuffling (collectively referred to as “DNA shuffling”). DNA shuffling involves the assembly of two or more DNA segments by homologous or site-specific recombination to generate variation in the polynucleotide sequence. DNA shuffling may be employed to modulate the activities of polypeptides of the invention, such methods can be used to generate polypeptides with altered activity. See, generally, U.S. Pat. Nos. 5,605,793; 5,811,238; 5,830,721; 5,834,252; 5,837,458; and 6,444,468; and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998). Thus, one or more components, motifs, sections, parts, domains, fragments, etc., of a polypeptide of the invention may be joined to one or more components, motifs, sections, parts, domains, fragments, etc. of one or more heterologous molecules, preferably the polymerases in SEQ ID NOS:27-34 and/or of SEQ ID NOS:14-25.

Polypeptides comprising fragments, mutants, variants, or full length polypeptides of the invention may be “free-standing,” or comprised within a larger polypeptide of which the fragment, mutant, variant, or full length polypeptide forms a part or region.

Thus, the polypeptides may include one or more additional amino acids and/or one or more heterologous sequences such as those described herein. For instance, a methionine residue may be added to the N-terminus of the polypeptide to allow for recombinant expression. Also, a sequence of additional amino acids, particularly charged amino acids, may be added to the N-terminus of the polypeptide to improve stability and persistence, in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another protein or part thereof. For some uses it would be desirable to be able to remove the Fc part after the fusion protein has been expressed, detected and purified in the advantageous manner described. This is the case when Fc portion proves to be a hindrance, for example when the fusion protein is to be used as an immunogen for raising antibodies. In drug discovery, for example, human proteins, such as hIL5-receptor, have been fused with Fc portions for the purpose of high-throughput screening assays to identify antagonists of hIL-5. See, D. Bennett et al., Journal of Molecular Recognition, Vol. 8:52-58 (1995) and K. Johanson et al., The Journal of Biological Chemistry, Vol. 270, No. 16:9459-9471 (1995).

Thus, the polypeptides may be in the form of the secreted protein, including a mature form, or may be a part of a larger protein, such as a fusion protein. It is often advantageous to include an additional amino acid(s), preferably a sequence which contains secretory or leader sequences, pro-sequences, sequences which aid in purification, such as multiple histidine residues, or an additional sequence for stability during recombinant production.

The polypeptides may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one which is fused with another compound, such as polyethylene glycol, or (iv) one which is fused to a heterologous sequence such as additional amino acids which aid in purification or which enhance processivity. Such polypeptides are deemed to be within the scope of those skilled in the art from the teachings herein.

Preferably, the polypeptides of the invention, including mutants, fragments and variants, demonstrate a functional activity such as an enzymatic activity described above (e.g., a DNA polymerase activity such as DNA-dependent DNA polymerase activity and/or reverse transriptase activity) or antigenicity.

The functional activity of polypeptides of the invention can be assayed by various methods. For example, in one embodiment where one is assaying for antigenicity, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In addition, assays described herein and otherwise known in the art may routinely be applied to measure the ability of polypeptides of the invention to elicit an enzymatic activity.

In some embodiments, the present invention provides polypeptides expressed from clones containing sequences encoding the polypeptides. The polypeptides may be expressed as native polypeptides, i.e., without any modifications to the primary sequence. Polypeptides may also be expressed as fusion proteins (e.g., N-terminal and/or C-terminal) and/or may be post-translationally modified (e.g., glycosylated, etc.).

In some embodiments, the polypeptides expressed from nucleic acids of the present invention may be modified to contain a tag (e.g., an affinity tag) in order to facilitate the purification of the polypeptide. Suitable tags are well known to those skilled in the art and include, but are not limited to, repeated sequences of amino acids such as six histidines, epitopes such as the hemagglutinin epitope, the V5 epitope, and the myc epitope, and other amino acid sequences that permit the simplified purification of the polypeptide. For example, the vectors used to clone the polyps of the invention contain the amino acid sequence of the PelB leader, which directs periplasmic localization of polypeptides. The present invention also contemplates polypeptides that do not contain a tag sequence. The sequences in SEQ ID NOS:14-25, which include a tag sequence, may be used to construct vectors expressing un-tagged versions of the polypeptides. The present invention also encompasses these un-tagged proteins and the nucleic acid that encode them.

The invention further relates to fusion proteins comprising (1) a polypeptide, or fragment thereof, having one or more desired characteristics and/or activities and (2) a tag (e.g., an affinity tag), as well as nucleic acid molecules that encode such fusion proteins. In particular embodiments, the invention includes a polypeptide described herein having one or more (e.g., one, two, three, four, five, six, seven, eight, etc.) tags. These tags may be located, for example, (1) at the N-terminus, (2) at the C-terminus, or (3) at both the N-terminus and C-terminus of the protein, or a fragment thereof having one or more desired characteristic and/or activity. A tag may also be located internally (e.g., between regions of amino acid sequence of a polypeptide of the invention).

Tags used in the invention may vary in length but will typically be from about 5 to about 100, from about 10 to about 100, from about 15 to about 100, from about 20 to about 100, from about 25 to about 100, from about 30 to about 100 from about 35 to about 100, from about 40 to about 100, from about 45 to about 100, from about 50 to about 100, from about 55 to about 100, from about 60 to about 100, from about 65 to about 100, from about 70 to about 100, from about 75 to about 100, from about 80 to about 100, from about 85 to about 100, from about 90 to about 100, from about 95 to about 100, from about 5 to about 80, from about 10 to about 80, from about 20 to about 80, from about 30 to about 80, from about 40 to about 80, from about 50 to about 80, from about 60 to about 80, from about 70 to about 80, from about 5 to about 60, from about 10 to about 60, from about 20 to about 60, from about 30 to about 60, from about 40 to about 60, from about 50 to about 60, from about 5 to about 40, from about 10 to about 40, from about 20 to about 40, from about 30 to about 40, from about 5 to about 30, from about 10 to about 30, from about 20 to about 30, from about 5 to about 25, from about 10 to about 25, or from about 15 to about 25 amino acid residues in length.

Tags used in the practice of the invention may serve any number of purposes. For example, such tags may (1) contribute to protein-protein interactions both internally within a protein (e.g., between a tag sequence and a polypeptide sequence to which the tag has been attached) and with other protein molecules, (2) make the polypeptide amenable to particular purification methods (e.g., affinity purification), (3) enable one to identify whether the polypeptide is present in a composition (e.g. ELISA, Western blot, etc.), and/or (4) stabilize or destabilize intra-protein interactions with the protein to which the tag has been added (e.g., increase or decrease thermostability of the protein).

Examples of tags which may be used in the practice of the invention include metal binding domains (e.g., a poly-histidine segments such as a three, four, five, six, or seven histidine region), immunoglobulin binding domains (e.g., (1) Protein A; (2) Protein G; (3) T cell, B cell, and/or Fc receptors; and/or (4) complement protein antibody-binding domain); sugar binding domains (e.g., a maltose binding domain); and detectable domains (e.g., at least a portion of .beta.-galactosidase). Fusion proteins may contain one or more tags such as those described above. Typically, fusion proteins that contain more than one tag will contain these tags at one terminus or both termini (i.e., the N-terminus and the C-terminus) of the polypeptide, although one or more tags may be located internally in addition to those present at the termini. Further, more than one tag may be present at one terminus, internally and/or at both termini of the polypeptide. For example, three consecutive tags could be linked end-to-end at the N-terminus of the polypeptide. The invention further includes compositions and reaction mixture that contain the above fusion proteins, as well as methods for preparing these fusion proteins, nucleic acid molecules (e.g., vectors) which encode these fusion proteins and recombinant host cells that contain these nucleic acid molecules. The invention also includes methods for using these fusion proteins as described elsewhere herein.

Tags that enable one to identify whether the fusion protein is present in a composition include, for example, tags that can be used to identify the protein in an electrophoretic gel. A number of such tags are known in the art and include epitopes and antibody binding domains, which can be used for Western blots.

In some embodiments, it may be desirable to remove all or a portion of a tag sequence from a fusion protein comprising a tag sequence and a polypeptide of the invention. In embodiments of this type, one or more amino acids forming a cleavage site, e.g., for a protease enzyme, may be incorporated into the primary sequence of the fusion protein. The cleavage site may be located such that cleavage at the site may remove all or a portion of the tag sequence from the fusion protein. In some embodiments, the cleavage site may be located between the tag sequence and the sequence of the polypeptide such that all of the tag sequence is removed by cleavage with a protease enzyme that recognizes the cleavage site. Examples of suitable cleavage sites include, but are not limited to, the Factor Xa cleavage site having the sequence Ile-Glu-Gly-Arg (SEQ ID NO:35), which is recognized and cleaved by blood coagulation factor Xa, and the thrombin cleavage site having the sequence Leu-Val-Pro-Arg (SEQ ID NO:36), which is recognized and cleaved by thrombin. Other suitable cleavage sites are known to those skilled in the art and may be used in conjunction with the present invention.

### 2. Nucleic Acid Molecules of the Invention

This invention also relates to nucleic acids that encode or are complementary a nucleic acid encoding a polypeptide of the invention. These nucleic acids can then be used to produce the polypeptide in recombinant cell culture. In still other aspects, the invention provides an isolated nucleic acid molecule encoding polypeptide of the invention, either labeled or unlabeled, or a nucleic acid sequence that is complementary to, or hybridizes under stringent conditions to, a nucleic acid sequence encoding a polypeptide of the invention.

Using the information provided herein, such as all or a portion of the nucleotide sequences in any one of SEQ ID NOS:2-13, a nucleic acid molecule of the present invention encoding a polypeptide of the invention may be obtained using standard cloning and screening procedures, such as those for cloning cDNAs using mRNA as starting material and/or those for screening a genomic library.

Nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment. For example, recombinant DNA molecules contained in vectors are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising all or a portion of an open reading frame (ORF) shown in one or more of SEQ ID NOs: 14-25.

The present invention is further directed to fragments of the isolated nucleic acid molecules described herein. Preferred nucleic acid fragments of the present invention include nucleic acid molecules encoding one or more portions (e.g., domains) of a polypeptide of the invention having one or more activities (e.g., enzymatic activities such as enzymatic activities discussed herein). In particular, such nucleic acid fragments of the present invention include nucleic acid molecules encoding polypeptides having RNA-dependent DNA polymerase activity.

In another aspect, the invention provides an isolated nucleic acid molecule comprising a polynucleotide that hybridizes under stringent hybridization conditions to all or a portion of a polynucleotide encoding a polypeptide of the invention. By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of a reference polynucleotide (e.g., one or more of SEQ ID NOS: 2-13). Preferably, a polynucleotide that hybridizes under stringent hybridization conditions to all or a portion of a reference sequence encodes a polypeptide having one or more enzymatic activities such as an enzymatic activity discussed herein (e.g., an RNA-dependent DNA polymerase activity).

Nucleic acid molecules of the present invention that encode a polypeptide of the invention may include, but are not limited to, those encoding the amino acid sequence of the polypeptide, by itself; the coding sequence for the polypeptide and additional sequences, such as those encoding a leader or secretory sequence, such as a pre-, or pro- or prepro-protein sequence; the coding sequence of the polypeptide, with or without the aforementioned additional coding sequences, together with additional, non-coding sequences, including for example, but not limited to non-coding 5′ and 3′ sequences, such as the transcribed, non-translated sequences that play a role in transcription, mRNA processing, including splicing and polyadenylation signals, for example—ribosome binding and stability of mRNA. Nucleic acid molecules of the invention include those encoding a polypeptide of the invention and comprising at least one additional coding sequences that codes for one or more of the tag sequences discussed above.

The present invention further relates to variants of the nucleic acid molecules of the present invention that encode portions, analogs or derivatives of the polypeptides of the invention. Variants may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985).

Non-naturally occurring variants may be produced using art-known mutagenesis techniques. Such variants include those produced by nucleotide substitutions, deletions or additions which may involve one or more nucleotides. The variants may be altered in coding regions, non-coding regions, or both. Alterations in the coding regions may produce conservative or non-conservative amino acid substitutions, deletions or additions.

Further embodiments of the invention include isolated nucleic acid molecules comprising a polynucleotide having a nucleotide sequence at least 90% identical, and more preferably at least 95%, 96%, 97%, 98% or 99% identical to (a) a nucleotide sequence encoding a polypeptide having all or a portion of the amino acid sequence in any one of SEQ ID NOS:14-25 and (b) a nucleotide sequence complementary to any of the nucleotide sequences in (a).

Polynucleotides of the invention include, but are not limited to, polynucleotides comprising, or alternatively consisting of, a nucleic acid encoding a polypeptide of SEQ ID NOS:14-25, polynucleotides comprising, or alternatively consisting of, a nucleotide sequence of SEQ ID NOS: 2-13, polynucleotides comprising, or alternatively consisting of, a nucleic acid encoding a polypeptide encoded by a nucleotide sequence of one of the deposited clones (NRRL Deposit Numbers NRRL B-30617, NRRL B-30618, NRRL B-30619, NRRL B-30620, NRRL B-30621, NRRL B-30622, NRRL B-30623, NRRL B-30624, NRRL B-30625, NRRL B-30626, NRRL B-30576, NRRL B-30577, NRRL B-30579, NRRL B-30578, NRRL B-30580), polynucleotides comprising, or alternatively consisting of, a nucleotide sequence of one of the deposited clones (NRRL Deposit Numbers NRRL B-30617, NRRL B-30618, NRRL B-30619, NRRL B-30620, NRRL B-30621, NRRL B-30622, NRRL B-30623, NRRL B-30624, NRRL B-30625, NRRL B-30626, NRRL B-30576, NRRL B-30577, NRRL B-30579, NRRL B-30578, NRRL B-30580), and/or mutants, fragments (e.g., portions), and variants thereof.

As described above, and further described below, polynucleotides of the invention also include, but are not limited to, polynucleotides comprising, or alternatively consisting of, nucleic acids encoding a mutant polymerases which comprise one or more substitutions corresponding to an amino acid residue of an amino acid sequence of SEQ ID NOS:14-25, polynucleotides comprising, or alternatively consisting of, nucleic acids which comprise one or more substitutions corresponding to a nucleotide sequence of SEQ ID NOS:2-13, polynucleotides comprising, or alternatively consisting of, nucleic acids encoding mutant polymerases which comprise one or more substitutions corresponding to an amino acid residue of a polypeptide encoded by a nucleotide sequence of one of the deposited clones (NRRL Deposit Numbers NRRL B-30617, NRRL B-30618, NRRL B-30619, NRRL B-30620, NRRL B-30621, NRRL B-30622, NRRL B-30623, NRRL B-30624, NRRL B-30625, NRRL B-30626, NRRL B-30576, NRRL B-30577, NRRL B-30579, NRRL B-30578, NRRL B-30580), polynucleotides comprising, or alternatively consisting of, nucleic acids which comprise one or more substitutions corresponding to a nucleotide sequence of one of the deposited clones (NRRL Deposit Numbers NRRL B-30617, NRRL B-30618, NRRL B-30619, NRRL B-30620, NRRL B-30621, NRRL B-30622, NRRL B-30623, NRRL B-30624, NRRL B-30625, NRRL B-30626, NRRL B-30576, NRRL B-30577, NRRL B-30579, NRRL B-30578, NRRL B-30580) and/or mutants, fragments (e.g., portions), and variants thereof.

SEQ ID NOS: 2-13 and the corresponding translated SEQ ID NOS:14-25 are sufficiently accurate and otherwise suitable for a variety of uses well known in the art and described further below. For instance, SEQ ID NOS:2-13 are useful for designing nucleic acid hybridization probes/primers that will detect and/or amplify nucleic acid sequences contained in SEQ ID NOS:2-13, respectively, or the DNAs contained in the respective deposited clone. These probes/primers will also hybridize to/amplify nucleic acid molecules in microbiological samples, thereby enabling detection of the respective organism from which SEQ ID NOS: 2-13 are derived. Similarly, polypeptides identified from SEQ ID NOS:14-25 may be used, for example, to generate antibodies which bind specifically to the polypeptides of the invention.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NOS: 2-13 and the predicted corresponding translated amino acid sequences identified as SEQ ID NOS:14-25, but also a sample of plasmid DNA containing a DNA clone the polymerases of the invention deposited with the NRRL depository (see examples). The nucleotide sequence of the deposited clones can readily be determined by sequencing the deposited clones in accordance with known methods. The predicted amino acid sequences can then be verified from such deposits. Moreover, the amino acid sequence of the protein encoded by the deposited clone can also be directly determined by peptide sequencing or by expressing the protein in a suitable host cell containing the deposited DNA, collecting the protein, and determining its sequence.

The polynucleotides of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand.

Nucleic acids encoding a polypeptide of SEQ ID NOS:14-25 may substantially differ from the nucleotide sequences in SEQ ID NOS: 2-13 or in the deposited clones due to the degeneracy of the genetic code. Of course, the genetic code is well known in the art. Thus, it would be routine for one skilled in the art to generate the degenerate polynucleotides described above.

The present invention particularly relates to polynucleotides which hybridize under stringent conditions to the hereinabove-described polynucleotides. The polynucleotides which hybridize to the hereinabove described polynucleotides in a preferred embodiment encode polypeptides which retain substantially the same functional activity as the polypeptide encoded by the nucleotide sequence of SEQ ID NOS: 2-13 or the polymerases encoded by the deposited clones.

In another aspect, the invention provides an isolated nucleic acid molecule comprising, or alternatively consisting of, a polynucleotide which hybridizes under stringent hybridization conditions to a portion of the polynucleotide in a nucleic acid molecule of the invention described above.

Such hybridizing polynucleotides may not encode a polypeptide, and are still useful, for example, as probes or primers.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of the reference polynucleotide. Also intended is a polynucleotide hybridizing to at least about 15 nucleotides (nt), and more preferably at least about 20 nt, more preferably at least about 25 nt, still more preferably at least about 30 nt, and even more preferably about 30-70 (e.g., 30, 35, 40, 45, 50, 55, 60, 65, and/or 70 (of course, fragment lengths in addition to those recited herein are also useful)) nt of the reference polynucleotide. Alternatively, the polynucleotide may have at least 20 bases, preferably 30 bases, and more preferably at least 50 bases which hybridize to a polynucleotide of the present invention, as hereinabove described, and which may or may not encode a polypeptide. Of course, larger fragments 50-500 nt, 500-1000 nt, 1000-1500 nt, 1500-2000 nt, 2000-2500 nt, 2500-3000 nt, 3000-3500 nt in length are also useful in the present invention (see below). For example, such polynucleotides may be employed as probes for the full length polynucleotides, for example, for recovery or detection of the polynucleotide or as a PCR primer.

Of course, polynucleotides hybridizing to a larger portion of the reference polynucleotide (e.g. the deposited cDNA clone) or even to the entire length of the reference polynucleotide, are also useful as probes according to the present invention, as are polynucleotides corresponding to most, if not all, of the nucleotide sequence of the deposited clone or the nucleotide sequence as shown in SEQ ID NOS: 2-13. By a portion of a polynucleotide of “at least 20 nt in length,” for example, is intended 20 or more contiguous nucleotides from the nucleotide sequence of the reference polynucleotide. As indicated, such portions are useful as a probe according to conventional DNA hybridization techniques or as primers for amplification of a target sequence by the polymerase chain reaction (PCR), as described herein.

Generating polynucleotides which hybridize to a portion of the nucleic acid molecules would be routine to the skilled artisan. For example, restriction endonuclease cleavage or shearing by sonication of a deposited clone could easily be used to generate DNA portions of various sizes which are polynucleotides that hybridize to a portion of the full length nucleic acid molecule. Alternatively, the hybridizing polynucleotides of the present invention could be generated synthetically according to known techniques.

The present invention is further directed to fragments of the isolated nucleic acid molecules described herein. By a fragment of an isolated nucleic acid molecule having the nucleotide sequence of a deposited cone, or a nucleotide sequence shown in SEQ ID NOS: 2-13 is intended fragments of at least about 15 nucleotides (nt), and more preferably at least about 20 nt, still more preferably at least about 30 nt, and even more preferably, at least about 40 nt in length which are useful as probes and primers as discussed herein. Of course, larger fragments 50-500 nt, 500-1000 nt, 1000-1500 nt, 1500-2000 nt, 2000-2500 nt, 2500-3000 nt, 3000-3500 nt in length are also useful according to the present invention as are fragments corresponding to most, if not all, of a nucleotide sequence of a deposited clone, or as shown in SEQ ID NOS: 2-13. By a fragment at least 20 nt in length, for example, is intended fragments which include 20 or more contiguous bases from the nucleotide sequence of a deposited clone or the nucleotide sequence as shown in SEQ ID NOS: 2-13.

Polynucleotide fragments and hybridizing polynucleotides may be any length from 15 to 4000 nucleotides in length.

Polynucleotides of the invention include variants which are at least 80%, 81%, 82%, 83%, 84%, 85%, 86%, 87%, 88%, 89%, 90%, 91%, 92%, 93%, 94%, 95% 96%, 97%, 98%, or 99% identical to the polypeptide-encoding or polymerase-encoding nucleotide sequences of SEQ ID NOS:2-13, or to the polymerase nucleic acids of the deposited clones, or to the polynucleotide fragments described above.

Thus, the invention includes, in part, polynucleotides which are at least 80%, 81%, 82%, 83%, 84%, 85%, 86%, 87%, 88%, 89%, 90%, 91%, 92%, 93%, 94%, 95% 96%, 97%, 98%, or 99% identical to (1) nucleic acid contained in a deposited clone described herein, (2) to a polynucleotide having a nucleotide sequence set out in SEQ ID NOS:2-13, or (3) to a subportion of one of these polynucleotides (e.g., nucleotides 225-398, 156-402, 450-779, 459-2201 of SEQ ID NO:2). The invention further includes host cells which contain such nucleic acid molecules. The invention also includes compositions and mixtures (e.g., reaction mixtures) which contain one or more of these polynucleotides, as well as methods for producing polypeptides using these polynucleotides.

In many instances, the above described polynucleotides will encode polypeptides which have one or more activity associated with a polypeptide encoded by a deposited clone described herein or a polypeptide having an amino acid sequence set out in SEQ ID NOS:14-25.

The variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons, e.g., to optimize codon expression for a particular host (change codons to those preferred by a particular bacterial host such as E. coli). Most highly preferred are nucleic acid molecules encoding an amino acid sequence encoded by a deposited clone, as described herein. Isolated nucleic acid molecules, particularly DNA molecules, are useful as probes and primers for producing the polypeptides of the invention, for example, by PCR or DNA shuffling.

Polynucleotides of the invention include polynucleotides comprising or consisting of nucleic acids encoding fragments of the polypeptides of SEQ ID NOS:14-25 or the polymerases encoded by the deposited clones.

Nucleic acids may encode fragments which are from 6 to 994 amino acids in length.

Nucleic acids may encode fragments which are 10 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 10 amino acids in length such as residues 1-10, 2-11, 3-12, . . . , 911-920 of the polypeptide or polymerase of SEQ ID NO:14; residues 1-10, 2-11, 3-12, . . . , 880-889 of the polypeptide or polymerase of SEQ ID NO:15; residues 1-10, 2-11, 3-12, . . . , 916-925 of the polypeptide or polymerase of SEQ ID NO:16; residues 1-10, 2-11, 3-12, . . . , 862-871 of the polypeptide or polymerase of SEQ ID NO:17; residues 1-10, 2-11, 3-12, . . . , 862-871 of the polypeptide or polymerase of SEQ ID NO:18; residues 1-10, 2-11, 3-12, . . . , 862-871 of the polypeptide or polymerase of SEQ ID NO:19; residues 1-10, 2-11, 3-12, . . . , 891-900 of the polypeptide or polymerase of SEQ ID NO:20; residues 1-10, 2-11, 3-12, . . . , 855-864 of the polypeptide or polymerase of SEQ ID NO:21; residues 1-10, 2-11, 3-12, . . . , 875-884 of the polypeptide or polymerase of SEQ ID NO:22; residues 1-10, 2-11, 3-12, . . . , 861-870 of the polypeptide or polymerase of SEQ ID ID:23; residues 1-10, 2-11, 3-12, . . . , 919-928 of the polypeptide or polymerase of SEQ ID NO:24; residues 1-10, 2-11, 3-12, . . . , 951-960 of the polypeptide or polymerase of SEQ ID NO:25.

Nucleic acids may encode fragments which are 11 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 11 amino acids in length such as amino acid residues 1-11, 2-12, 3-13, . . . , 910-920 of the polypeptide or polymerase of SEQ ID NO:14; residues 1-11, 2-12, 3-13, . . . , 879-889 of the polypeptide or polymerase of SEQ ID NO:15; residues 1-11, 2-12, 3-13, . . . , 915-925 of the polypeptide or polymerase of SEQ ID NO:16; residues 1-11, 2-12, 3-13, . . . , 861-871 of the polypeptide or polymerase of SEQ ID NO:17; residues 1-11, 2-12, 3-13, . . . , 861-871 of the polypeptide or polymerase of SEQ ID NO:18; residues 1-11, 2-12, 3-13, . . . , 861-871 of the polypeptide or polymerase of TablSEQ ID NO:19; residues 1-11, 2-12, 3-13, . . . , 890-900 of the polypeptide or polymerase of SEQ ID NO:20; residues 1-11, 2-12, 3-13, . . . , 854-864 of the polypeptide or polymerase of SEQ ID NO:21; residues 1-11, 2-12, 3-13, . . . , 874-884 of the polypeptide or polymerase SEQ ID NO:22; residues 1-11, 2-12, 3-13, . . . , 860-870 of the polypeptide or polymerase of SEQ ID NO:23; residues 1-11, 2-12, 3-13, . . . , 918-928 of the polypeptide or polymerase of SEQ ID NO:24; residues 1-11, 2-12, 3-13, . . . , 950-960 of the polypeptide or polymerase of SEQ ID NO:25. An antibody of the invention may specifically bind one of the above fragments, or more than one fragments which overlap.

Nucleic acids may encode fragments which are 12 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 12 amino acids in length such as amino acid residues 1-12, 2-13, 3-14, . . . , 909-920 of the polypeptide or polymerase of SEQ ID NO:14; residues 1-12, 2-13, 3-14, . . . , 878-889 of the polypeptide or polymerase of SEQ ID NO:15; residues 1-12, 2-13, 3-14, . . . , 914-925 of the polypeptide or polymerase of SEQ ID NO:16; residues 1-12, 2-13, 3-14, . . . , 860-871 of the polypeptide or polymerase of SEQ ID NO:17; residues 1-12, 2-13, 3-14, . . . , 860-871 of the polypeptide or polymerase of SEQ ID NO:18; residues 1-12, 2-13, 3-14, . . . , 860-871 of the polypeptide or polymerase of SEQ ID NO:19; residues 1-12, 2-13, 3-14, . . . , 889-900 of the polypeptide or polymerase of SEQ ID NO:20; residues 1-12, 2-13, 3-14, . . . , 853-864 of the polypeptide or polymerase of SEQ ID NO:21; residues 1-12, 2-13, 3-14, . . . , 873-884 of the polypeptide or polymerase of SEQ ID NO:22; residues 1-12, 2-13, 3-14, . . . , 859-870 of the polypeptide or polymerase of SEQ ID NO:23; residues 1-12, 2-13, 3-14, . . . , 917-928 of the polypeptide or polymerase of SEQ ID NO:24; residues 1-12, 2-13, 3-14, . . . , 949-960 of the polypeptide or polymerase of SEQ ID NO:25.

Nucleic acids may encode fragments which are 13 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 13 amino acids in length such as amino acid residues 1-13, 2-14, 3-15, . . . , 908-920 of the polypeptide or polymerase of SEQ ID NO:14; residues 1-13, 2-14, 3-15, . . . , 877-889 of the polypeptide or polymerase of SEQ ID NO:15; residues 1-13, 2-14, 3-15, . . . , 913-925 of the polypeptide or polymerase of SEQ ID NO:16; residues 1-13, 2-14, 3-15, . . . , 859-871 of the polypeptide or polymerase of SEQ ID NO:17; residues 1-13, 2-14, 3-15, . . . , 859-871 of the polypeptide or polymerase of SEQ ID NO:18; residues 1-13, 2-14, 3-15, . . . , 859-871 of the polypeptide or polymerase of SEQ ID NO:19; residues 1-13, 2-14, 3-15, . . . , 888-900 of the polypeptide or polymerase of SEQ ID NO:20; residues 1-13, 2-14, 3-15, . . . , 852-864 of the polypeptide or polymerase of SEQ ID NO:21; residues 1-13, 2-14, 3-15, . . . , 872-884 of the polypeptide or polymerase of SEQ ID NO:22; residues 1-13, 2-14, 3-15, . . . , 858-870 of the polypeptide or polymerase of SEQ ID NO:23; residues 1-13, 2-14, 3-15, . . . , 916-928 of the polypeptide or polymerase of SEQ ID NO:24; residues 1-13, 2-14, 3-15, . . . , 948-960 of the polypeptide or polymerase of SEQ ID NO:25.

Nucleic acids may encode fragments which are 14 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 14 amino acids in length such as amino acid residues 1-14, 2-15, 3-16, . . . , 907-920 of the polypeptide or polymerase of SEQ ID NO:14; residues 1-14, 2-15, 3-16, . . . , 876-889 of the polypeptide or polymerase of SEQ ID NO:15; residues 1-14, 2-15, 3-16, . . . , 912-925 of the polypeptide or polymerase of SEQ ID NO:16; residues 1-14, 2-15, 3-16, . . . , 858-871 of the polypeptide or polymerase of SEQ ID NO:17; residues 1-14, 2-15, 3-16, . . . , 858-871 of the polypeptide or polymerase of SEQ ID NO:18; residues 1-14, 2-15, 3-16, . . . , 858-871 of the polypeptide or polymerase of SEQ ID NO:19; residues 1-14, 2-15, 3-16, . . . , 887-900 of the polypeptide or polymerase of SEQ ID NO:20; residues 1-14, 2-15, 3-16, . . . , 851-864 of the polypeptide or polymerase of SEQ ID NO:21; residues 1-14, 2-15, 3-16, . . . , 871-884 of the polypeptide or polymerase of SEQ ID NO:22; residues 1-14, 2-15, 3-16, . . . , 857-870 of the polypeptide or polymerase of SEQ ID NO:23; residues 1-14, 2-15, 3-16, . . . , 915-928 of the polypeptide or polymerase of SEQ ID NO:24; residues 1-14, 2-15, 3-16, . . . , 947-960 of the polypeptide or polymerase of SEQ ID NO:25.

Nucleic acids may encode fragments which are 15 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 15 amino acids in length such as amino acid residues 1-15, 2-16, 3-17, . . . , 906-920 of the polypeptide or polymerase of SEQ ID NO:14; residues 1-15, 2-16, 3-17, . . . , 875-889 of the polypeptide or polymerase of SEQ ID NO:15; residues 1-15, 2-16, 3-17, . . . , 911-925 of the polypeptide or polymerase of SEQ ID NO:16; residues 1-15, 2-16, 3-17, . . . , 857-871 of the polypeptide or polymerase of SEQ ID NO:17; residues 1-15, 2-16, 3-17, . . . , 857-871 of the polypeptide or polymerase of SEQ ID NO:18; residues 1-15, 2-16, 3-17, . . . , 857-871 of the polypeptide or polymerase of SEQ ID NO:19; residues 1-15, 2-16, 3-17, . . . , 886-900 of the polypeptide or polymerase of SEQ ID NO:20; residues 1-15, 2-16, 3-17, . . . , 850-864 of the polypeptide or polymerase of SEQ ID NO:21; residues 1-15, 2-16, 3-17, . . . , 870-884 of the polypeptide or polymerase of SEQ ID NO:22; residues 1-15, 2-16, 3-17, . . . , 856-870 of the polypeptide or polymerase of SEQ ID NO:23; residues 1-15, 2-16, 3-17, . . . , 914-928 of the polypeptide or polymerase of SEQ ID NO:24; residues 1-15, 2-16, 3-17, . . . , 946-960 of the polypeptide or polymerase of SEQ ID NO:25.

Nucleic acids may encode fragments which are 16 amino acids in length, and may begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 16 amino acids in length such as amino acid residues 1-16, 2-17, 3-18, . . . , 905-920 of the polypeptide or polymerase of SEQ ID NO:14; residues 1-16, 2-17, 3-18, . . . , 874-889 of the polypeptide or polymerase of SEQ ID NO:15; residues 1-16, 2-17, 3-18, . . . , 910-925 of the polypeptide or polymerase of SEQ ID NO:16; residues 1-16, 2-17, 3-18, . . . , 856-871 of the polypeptide or polymerase of SEQ ID NO:17; residues 1-16, 2-17, 3-18, . . . , 856-871 of the polypeptide or polymerase of SEQ ID NO:18; residues 1-16, 2-17, 3-18, . . . , 856-871 of the polypeptide or polymerase of SEQ ID NO:19; residues 1-16, 2-17, 3-18, . . . , 885-900 of the polypeptide or polymerase of SEQ ID NO:20; residues 1-16, 2-17, 3-18, . . . , 849-864 of the polypeptide or polymerase of SEQ ID NO:21; residues 1-16, 2-17, 3-18, . . . , 869-884 of the polypeptide or polymerase of SEQ ID NO:22; residues 1-16, 2-17, 3-18, . . . , 855-870 of the polypeptide or polymerase of SEQ ID NO:23; residues 1-16, 2-17, 3-18, . . . , 913-928 of the polypeptide or polymerase of SEQ ID NO:24; residues 1-16, 2-17, 3-18, . . . , 945-960 of the polypeptide or polymerase of SEQ ID NO:25.

Nucleic acids may encode fragments which are 17 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 17 amino acids in length such as amino acid residues 1-17, 2-18, 3-19, . . . , 904-920 of the polypeptide or polymerase of SEQ ID NO:14; residues 1-17, 2-18, 3-19, . . . , 873-889 of the polypeptide or polymerase of SEQ ID NO:15; residues 1-17, 2-18, 3-19, . . . , 909-925 of the polypeptide or polymerase of SEQ ID NO:16; residues 1-17, 2-18, 3-19, . . . , 855-871 of the polypeptide or polymerase of SEQ ID NO:17; residues 1-17, 2-18, 3-19, . . . , 855-871 of the polypeptide or polymerase of SEQ ID NO:18; residues 1-17, 2-18, 3-19, . . . , 855-871 of the polypeptide or polymerase of SEQ ID NO:19; residues 1-17, 2-18, 3-19, . . . , 884-900 of the polypeptide or polymerase of SEQ ID NO:20; residues 1-17, 2-18, 3-19, . . . , 848-864 of the polypeptide or polymerase of SEQ ID NO:21; residues 1-17, 2-18, 3-19, . . . , 868-884 of the polypeptide or polymerase of SEQ ID NO:22; residues 1-17, 2-18, 3-19, . . . , 854-870 of the polypeptide or polymerase of SEQ ID NO:23; residues 1-17, 2-18, 3-19, . . . , 912-928 of the polypeptide or polymerase of SEQ ID NO:24; residues 1-17, 2-18, 3-19, . . . , 944-960 of the polypeptide or polymerase of SEQ ID NO:25.

Nucleic acids may encode fragments which are 18 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 18 amino acids in length such as amino acid residues 1-18, 2-19, 3-20, . . . , 903-920 of the polypeptide or polymerase of SEQ ID NO:14; residues 1-18, 2-19, 3-20, . . . , 872-889 of the polypeptide or polymerase of SEQ ID NO:15; residues 1-18, 2-19, 3-20, . . . , 908-925 of the polypeptide or polymerase of SEQ ID NO:16; residues 1-18, 2-19, 3-20, . . . , 854-871 of the polypeptide or polymerase of SEQ ID NO:17; residues 1-18, 2-19, 3-20, . . . , 854-871 of the polypeptide or polymerase of SEQ ID NO:18; residues 1-18, 2-19, 3-20, . . . , 854-871 of the polypeptide or polymerase of SEQ ID NO:19; residues 1-18, 2-19, 3-20, . . . , 883-900 of the polypeptide or polymerase of SEQ ID NO:20; residues 1-18, 2-19, 3-20, . . . , 847-864 of the polypeptide or polymerase of SEQ ID NO:21; residues 1-18, 2-19, 3-20, . . . , 867-884 of the polypeptide or polymerase of SEQ ID NO:22; residues 1-18, 2-19, 3-20, . . . , 853-870 of the polypeptide or polymerase of SEQ ID NO:23; residues 1-18, 2-19, 3-20, . . . , 911-928 of the polypeptide or polymerase of SEQ ID NO:24; residues 1-18, 2-19, 3-20, . . . , 943-960 of the polypeptide or polymerase of SEQ ID NO:25.

Nucleic acids may encode fragments which are 19 amino acids in length, and begin at any amino acid residue 1 of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 19 amino acids in length such as amino acid residues 1-19, 2-20, 3-21, . . . , 902-920 of the polypeptide or polymerase of SEQ ID NO:14; residues 1-19, 2-20, 3-21, . . . , 871-889 of the polypeptide or polymerase of SEQ ID NO:15; residues 1-19, 2-20, 3-21, . . . , 907-925 of the polypeptide or polymerase of SEQ ID NO:16; residues 1-19, 2-20, 3-21, . . . , 853-871 of the polypeptide or polymerase of SEQ ID NO:17; residues 1-19, 2-20, 3-21, . . . , 853-871 of the polypeptide or polymerase of SEQ ID NO:18; residues 1-19, 2-20, 3-21, . . . , 853-871 of the polypeptide or polymerase of SEQ ID NO:19; residues 1-19, 2-20, 3-21, . . . , 882-900 of the polypeptide or polymerase of SEQ ID NO:20; residues 1-19, 2-20, 3-21, . . . , 846-864 of the polypeptide or polymerase of SEQ ID NO:21; residues 1-19, 2-20, 3-21, . . . , 866-884 of the polypeptide or polymerase of SEQ ID NO:22; residues 1-19, 2-20, 3-21, . . . , 852-870 of the polypeptide or polymerase of SEQ ID NO:23; residues 1-19, 2-20, 3-21, . . . , 910-928 of the polypeptide or polymerase of SEQ ID NO:24; residues 1-19, 2-20, 3-21, . . . , 942-960 of the polypeptide or polymerase of SEQ ID NO:25.

Nucleic acids may encode fragments which are 20 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 20 amino acids in length such as amino acid residues 1-20, 2-21, 3-22, . . . , 901-920 of the polypeptide or polymerase of SEQ ID NO:14; residues 1-20, 2-21, 3-22, . . . , 870-889 of the polypeptide or polymerase of SEQ ID NO:15; residues 1-20, 2-21, 3-22, . . . , 906-925 of the polypeptide or polymerase of SEQ ID NO:16; residues 1-20, 2-21, 3-22, . . . , 852-871 of the polypeptide or polymerase of SEQ ID NO:17; residues 1-20, 2-21, 3-22, . . . , 852-871 of the polypeptide or polymerase of SEQ ID NO:18; residues 1-20, 2-21, 3-22, . . . , 852-871 of the polypeptide or polymerase of SEQ ID NO:19; residues 1-20, 2-21, 3-22, . . . , 881-900 of the polypeptide or polymerase of SEQ ID NO:20; residues 1-20, 2-21, 3-22, . . . , 845-864 of the polypeptide or polymerase of SEQ ID NO:21; residues 1-20, 2-21, 3-22, . . . , 865-884 of the polypeptide or polymerase of SEQ ID NO:22; residues 1-20, 2-21, 3-22, . . . , 851-870 of the polypeptide or polymerase of SEQ ID NO:23; residues 1-20, 2-21, 3-22, . . . , 909-928 of the polypeptide or polymerase of SEQ ID NO:24; residues 1-20, 2-21, 3-22, . . . , 941-960 of the polypeptide or polymerase of SEQ ID NO:25.

Nucleic acids may encode fragments which are 21 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 21 amino acid in length such as amino acid residues 1-21, 2-22, 3-23, . . . , 900-920 of the polypeptide or polymerase of SEQ ID NO:14; residues 1-21, 2-22, 3-23, . . . , 869-889 of the polypeptide or polymerase of SEQ ID NO:15; residues 1-21, 2-22, 3-23, . . . , 905-925 of the polypeptide or polymerase of SEQ ID NO:16; residues 1-21, 2-22, 3-23, . . . , 851-871 of the polypeptide or polymerase of SEQ ID NO:17; residues 1-21, 2-22, 3-23, . . . , 851-871 of the polypeptide or polymerase of SEQ ID NO:18; residues 1-21, 2-22, 3-23, . . . , 851-871 of the polypeptide or polymerase of SEQ ID NO:19; residues 1-21, 2-22, 3-23, . . . , 880-900 of the polypeptide or polymerase of SEQ ID NO:20; residues 1-21, 2-22, 3-23, . . . , 844-864 of the polypeptide or polymerase of SEQ ID NO:21; residues 1-21, 2-22, 3-23, . . . , 864-884 of the polypeptide or polymerase of SEQ ID NO:22; residues 1-21, 2-22, 3-23, . . . , 850-870 of the polypeptide or polymerase of SEQ ID NO:23; residues 1-21, 2-22, 3-23, . . . , 908-928 of the polypeptide or polymerase of SEQ ID NO:24; residues 1-21, 2-22, 3-23, . . . , 940-960 of the polypeptide or polymerase of SEQ ID NO:25.

Nucleic acids may encode fragments which are 22 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 22 amino acids in length such as amino acid residues 1-22, 2-23, 3-24, . . . , 899-920 of the polypeptide or polymerase of SEQ ID NO:14; residues 1-22, 2-23, 3-24, . . . , 868-889 of the polypeptide or polymerase of SEQ ID NO:15; residues 1-22, 2-23, 3-24, . . . , 904-925 of the polypeptide or polymerase of SEQ ID NO:16; residues 1-22, 2-23, 3-24, . . . , 850-871 of the polypeptide or polymerase of SEQ ID NO:17; residues 1-22, 2-23, 3-24, . . . , 850-871 of the polypeptide or polymerase of SEQ ID NO:18; residues 1-22, 2-23, 3-24, . . . , 850-871 of the polypeptide or polymerase of SEQ ID NO:19; residues 1-22, 2-23, 3-24, . . . , 879-900 of the polypeptide or polymerase of SEQ ID NO:20; residues 1-22, 2-23, 3-24, . . . , 843-864 of the polypeptide or polymerase of SEQ ID NO:21; residues 1-22, 2-23, 3-24, . . . , 863-884 of the polypeptide or polymerase of SEQ ID NO:22; residues 1-22, 2-23, 3-24, . . . , 849-870 of the polypeptide or polymerase of SEQ ID NO:23; residues 1-22, 2-23, 3-24, . . . , 907-928 of the polypeptide or polymerase of SEQ ID NO:24; residues 1-22, 2-23, 3-24, . . . , 939-960 of the polypeptide or polymerase of SEQ ID NO:25.

Nucleic acids may encode fragments which are 23 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 23 amino acids in length such as amino acid residues 1-23, 2-24, 3-25, . . . , 898-920 of the polypeptide or polymerase of SEQ ID NO:14; residues 1-23, 2-24, 3-25, . . . , 867-889 of the polypeptide or polymerase of SEQ ID NO:15; residues 1-23, 2-24, 3-25, . . . , 903-925 of the polypeptide or polymerase of SEQ ID NO:16; residues 1-23, 2-24, 3-25, . . . , 849-871 of the polypeptide or polymerase of SEQ ID NO:17; residues 1-23, 2-24, 3-25, . . . , 849-871 of the polypeptide or polymerase of SEQ ID NO:18; residues 1-23, 2-24, 3-25, . . . , 849-871 of the polypeptide or polymerase of SEQ ID NO:19; residues 1-23, 2-24, 3-25, . . . , 878-900 of the polypeptide or polymerase of SEQ ID NO:20; residues 1-23, 2-24, 3-25, . . . , 842-864 of the polypeptide or polymerase of SEQ ID NO:21; residues 1-23, 2-24, 3-25, . . . , 862-884 of the polypeptide or polymerase of SEQ ID NO:22; residues 1-23, 2-24, 3-25, . . . , 848-870 of the polypeptide or polymerase of SEQ ID NO:23; residues 1-23, 2-24, 3-25, . . . , 906-928 of the polypeptide or polymerase of SEQ ID NO:24; residues 1-23, 2-24, 3-25, . . . , 938-960 of the polypeptide or polymerase of SEQ ID NO:25.

Nucleic acids may encode fragments which are 24 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 24 amino acids in length such as amino acid residues 1-23, 2-24, 3-25, . . . , 897-920 of the polypeptide or polymerase of SEQ ID NO:14; residues 1-23, 2-24, 3-25, . . . , 866-889 of the polypeptide or polymerase of SEQ ID NO:15; residues 1-23, 2-24, 3-25, . . . , 902-925 of the polypeptide or polymerase of SEQ ID NO:16; residues 1-23, 2-24, 3-25, . . . , 848-871 of the polypeptide or polymerase of SEQ ID NO:17; residues 1-23, 2-24, 3-25, . . . , 848-871 of the polypeptide or polymerase of SEQ ID NO:18; residues 1-23, 2-24, 3-25, . . . , 848-871 of the polypeptide or polymerase of SEQ ID NO:19; residues 1-23, 2-24, 3-25, . . . , 877-900 of the polypeptide or polymerase of SEQ ID NO:20; residues 1-23, 2-24, 3-25, . . . , 841-864 of the polypeptide or polymerase of SEQ ID NO:21; residues 1-23, 2-24, 3-25, . . . , 861-884 of the polypeptide or polymerase of SEQ ID NO:22; residues 1-23, 2-24, 3-25, . . . , 847-870 of the polypeptide or polymerase of SEQ ID NO:23; residues 1-23, 2-24, 3-25, . . . , 905-928 of the polypeptide or polymerase of SEQ ID NO:24; residues 1-23, 2-24, 3-25, . . . , 937-960 of the polypeptide or polymerase of SEQ ID NO:25.

Nucleic acids may encode fragments which are 25 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 25 amino acids in length such as amino acid residues 1-24, 2-25, 3-26, . . . , 896-920 of the polypeptide or polymerase of SEQ ID NO:14; residues 1-24, 2-25, 3-26, . . . , 865-889 of the polypeptide or polymerase of SEQ ID NO:15; residues 1-24, 2-25, 3-26, . . . , 901-925 of the polypeptide or polymerase of SEQ ID NO:16; residues 1-24, 2-25, 3-26, . . . , 847-871 of the polypeptide or polymerase of SEQ ID NO:17; residues 1-24, 2-25, 3-26, . . . , 847-871 of the polypeptide or polymerase of SEQ ID NO:18; residues 1-24, 2-25, 3-26, . . . , 847-871 of the polypeptide or polymerase of SEQ ID NO:19; residues 1-24, 2-25, 3-26, . . . , 876-900 of the polypeptide or polymerase of SEQ ID NO:20; residues 1-24, 2-25, 3-26, . . . , 840-864 of the polypeptide or polymerase of SEQ ID NO:21; residues 1-24, 2-25, 3-26, . . . , 860-884 of the polypeptide or polymerase of SEQ ID NO:22; residues 1-24, 2-25, 3-26, . . . , 846-870 of the polypeptide or polymerase of SEQ ID NO:23; residues 1-24, 2-25, 3-26, . . . , 904-928 of the polypeptide or polymerase of SEQ ID NO:24; residues 1-24, 2-25, 3-26, . . . , 936-960 of the polypeptide or polymerase of SEQ ID NO:25.

Nucleic acids may encode fragments which are 26 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 26 amino acids in length such as amino acid residues 1-25, 2-26, 3-27, . . . , 895-920 of the polypeptide or polymerase of SEQ ID NO:14; residues 1-25, 2-26, 3-27, . . . , 864-889 of the polypeptide or polymerase of SEQ ID NO:15; residues 1-25, 2-26, 3-27, . . . , 900-925 of the polypeptide or polymerase of SEQ ID NO:16; residues 1-25, 2-26, 3-27, . . . , 846-871 of the polypeptide or polymerase of SEQ ID NO:17; residues 1-25, 2-26, 3-27, . . . , 846-871 of the polypeptide or polymerase of SEQ ID NO:18; residues 1-25, 2-26, 3-27, . . . , 846-871 of the polypeptide or polymerase of SEQ ID NO:19; residues 1-25, 2-26, 3-27, . . . , 875-900 of the polypeptide or polymerase of SEQ ID NO:20; residues 1-25, 2-26, 3-27, . . . , 839-864 of the polypeptide or polymerase of SEQ ID NO:21; residues 1-25, 2-26, 3-27, . . . , 859-884 of the polypeptide or polymerase of SEQ ID NO:22; residues 1-25, 2-26, 3-27, . . . , 845-870 of the polypeptide or polymerase of SEQ ID NO:23; residues 1-25, 2-26, 3-27, . . . , 903-928 of the polypeptide or polymerase of SEQ ID NO:24; residues 1-25, 2-26, 3-27, . . . , 935-960 of the polypeptide or polymerase of SEQ ID NO:25.

Nucleic acids may encode fragments which are 27 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 27 amino acids in length such as amino acid residues 1-26, 2-27, 3-28, . . . , 894-920 of the polypeptide or polymerase of SEQ ID NO:14; residues 1-26, 2-27, 3-28, . . . , 863-889 of the polypeptide or polymerase of SEQ ID NO:15; residues 1-26, 2-27, 3-28, . . . , 899-925 of the polypeptide or polymerase of SEQ ID NO:16; residues 1-26, 2-27, 3-28, . . . , 845-871 of the polypeptide or polymerase of SEQ ID NO:17; residues 1-26, 2-27, 3-28, . . . , 845-871 of the polypeptide or polymerase of SEQ ID NO:18; residues 1-26, 2-27, 3-28, . . . , 845-871 of the polypeptide or polymerase of SEQ ID NO:19; residues 1-26, 2-27, 3-28, . . . , 874-900 of the polypeptide or polymerase of SEQ ID NO:20; residues 1-26, 2-27, 3-28, . . . , 838-864 of the polypeptide or polymerase of SEQ ID NO:21; residues 1-26, 2-27, 3-28, . . . , 858-884 of the polypeptide or polymerase of SEQ ID NO:22; residues 1-26, 2-27, 3-28, . . . , 844-870 of the polypeptide or polymerase of SEQ ID NO:23; residues 1-26, 2-27, 3-28, . . . , 902-928 of the polypeptide or polymerase of SEQ ID NO:24; residues 1-26, 2-27, 3-28, . . . , 934-960 of the polypeptide or polymerase of SEQ ID NO:25.

Nucleic acids may encode fragments which are 28 amino acids in length, and begin at any amino acid residue of the full length polypeptide or the full length polymerase (e.g., the polypeptides of SEQ ID NOS:14-25) with or without the N-terminal amino acids encoded by the vectors, or the polymerases encoded by the deposited clones). Thus, nucleic acids may encode fragments 28 amino acids in length such as amino acid residues 1-27, 2-28, 3-29, . . . , 893-920 of the polypeptide or polymerase of SEQ ID NO:14; residues 1-27, 2-28, 3-29, . . . , 862-889 of the polypeptide or polymerase of SEQ ID NO:15; residues 1-27, 2-28, 3-29, . . . , 898-925 of the polypeptide or polymerase of SEQ ID NO:16; residues 1-27, 2-28, 3-29, . . . , 844-871 of the polypeptide or polymerase of SEQ ID NO:17; residues 1-27, 2-28, 3-29, . . . , 844-871 of the polypeptide or polymerase of SEQ ID NO:18; residues 1-27, 2-28, 3-29, . . . , 844-871 of the polypeptide or polymerase of SEQ ID NO:19; residues 1-27, 2-28, 3-29, . . . , 873-900 of the polypeptide or polymerase of SEQ ID NO:20; residues 1-27, 2-28, 3-29, . . . , 837-864 of the polypeptide or polymerase of SEQ ID NO:21; residues 1-27, 2-28, 3-29, . . . , 857-884 of the polypeptide or polymerase of SEQ ID NO:22; residues 1-27, 2-28, 3-29, . . . , 843-870 of the polypeptide or polymerase of SEQ ID NO:23; residues 1-27, 2-28, 3-29, . . . , 901-928 of the polypeptide or polymerase of SEQ ID NO:24; residues 1-27, 2-28, 3-29, . . . , 933-960 of the polypeptide or polymerase of SEQ ID NO:25.

Nucleic acids may encode fragments which are 29 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 29 amino acids in length such as amino acid residues 1-28, 2-29, 3-30, . . . , 892-920 of the polypeptide or polymerase of SEQ ID NO:14; residues 1-28, 2-29, 3-30, . . . , 861-889 of the polypeptide or polymerase of SEQ ID NO:15; residues 1-28, 2-29, 3-30, . . . , 897-925 of the polypeptide or polymerase of SEQ ID NO:16; residues 1-28, 2-29, 3-30, . . . , 843-871 of the polypeptide or polymerase of SEQ ID NO:17; residues 1-28, 2-29, 3-30, . . . , 843-871 of the polypeptide or polymerase of SEQ ID NO:18; residues 1-28, 2-29, 3-30, . . . , 843-871 of the polypeptide or polymerase of SEQ ID NO:19; residues 1-28, 2-29, 3-30, . . . , 872-900 of the polypeptide or polymerase of SEQ ID NO:20; residues 1-28, 2-29, 3-30, . . . , 836-864 of the polypeptide or polymerase of SEQ ID NO:21; residues 1-28, 2-29, 3-30, . . . , 856-884 of the polypeptide or polymerase of SEQ ID NO:22; residues 1-28, 2-29, 3-30, . . . , 842-870 of the polypeptide or polymerase of SEQ ID NO:23; residues 1-28, 2-29, 3-30, . . . , 900-928 of the polypeptide or polymerase of SEQ ID NO:24; residues 1-28, 2-29, 3-30, . . . , 932-960 of the polypeptide or polymerase of SEQ ID NO:25.

Nucleic acids may encode fragments which are 30 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 30 amino acids in length such as amino acid residues 1-29, 2-30, 3-31, . . . , 891-920 of the polypeptide or polymerase of SEQ ID NO:14; residues 1-29, 2-30, 3-31, . . . , 860-889 of the polypeptide or polymerase of SEQ ID NO:15; residues 1-29, 2-30, 3-31, . . . , 896-925 of the polypeptide or polymerase of SEQ ID NO:16; residues 1-29, 2-30, 3-31, . . . , 842-871 of the polypeptide or polymerase of SEQ ID NO:17; residues 1-29, 2-30, 3-31, . . . , 842-871 of the polypeptide or polymerase of SEQ ID NO:18; residues 1-29, 2-30, 3-31, . . . , 842-871 of the polypeptide or polymerase of SEQ ID NO:19; residues 1-29, 2-30, 3-31, . . . , 871-900 of the polypeptide or polymerase of SEQ ID NO:20; residues 1-29, 2-30, 3-31, . . . , 835-864 of the polypeptide or polymerase of SEQ ID NO:21; residues 1-29, 2-30, 3-31, . . . , 855-884 of the polypeptide or polymerase of SEQ ID NO:22; residues 1-29, 2-30, 3-31, . . . , 841-870 of the polypeptide or polymerase of SEQ ID NO:23; residues 1-29, 2-30, 3-31, . . . , 899-928 of the polypeptide or polymerase of SEQ ID NO:24; residues 1-29, 2-30, 3-31, . . . , 931-960 of the polypeptide or polymerase of SEQ ID NO:25.

Nucleic acids may encode fragments which are 31 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 31 amino acids in length such as amino acid residues 1-30, 2-31, 3-32, . . . , 890-920 of the polypeptide or polymerase of SEQ ID NO:14; residues 1-30, 2-31, 3-32, . . . , 859-889 of the polypeptide or polymerase of SEQ ID NO:15; residues 1-30, 2-31, 3-32, . . . , 895-925 of the polypeptide or polymerase of SEQ ID NO:16; residues 1-30, 2-31, 3-32, . . . , 841-871 of the polypeptide or polymerase of SEQ ID NO:17; residues 1-30, 2-31, 3-32, . . . , 841-871 of the polypeptide or polymerase of SEQ ID NO:18; residues 1-30, 2-31, 3-32, . . . , 841-871 of the polypeptide or polymerase of SEQ ID NO:19; residues 1-30, 2-31, 3-32, . . . , 870-900 of the polypeptide or polymerase of SEQ ID NO:20; residues 1-30, 2-31, 3-32, . . . , 834-864 of the polypeptide or polymerase of SEQ ID NO:21; residues 1-30, 2-31, 3-32, . . . , 854-884 of the polypeptide or polymerase of SEQ ID NO:22; residues 1-30, 2-31, 3-32, . . . , 840-870 of the polypeptide or polymerase of SEQ ID NO:23; residues 1-30, 2-31, 3-32, . . . , 898-928 of the polypeptide or polymerase of SEQ ID NO:24; residues 1-30, 2-31, 3-32, . . . , 930-960 of the polypeptide or polymerase of SEQ ID NO:25.

Nucleic acids may encode fragments which are 32 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 32 amino acids in length such as amino acid residues 1-31, 2-32, 3-33, . . . , 889-920 of the polypeptide or polymerase of SEQ ID NO:14; residues 1-31, 2-32, 3-33, . . . , 858-889 of the polypeptide or polymerase of SEQ ID NO:15; residues 1-31, 2-32, 3-33, . . . , 894-925 of the polypeptide or polymerase of SEQ ID NO:16; residues 1-31, 2-32, 3-33, . . . , 840-871 of the polypeptide or polymerase of SEQ ID NO:17; residues 1-31, 2-32, 3-33, . . . , 840-871 of the polypeptide or polymerase of SEQ ID NO:18; residues 1-31, 2-32, 3-33, . . . , 840-871 of the polypeptide or polymerase of SEQ ID NO:19; residues 1-31, 2-32, 3-33, . . . , 869-900 of the polypeptide or polymerase of SEQ ID NO:20; residues 1-31, 2-32, 3-33, . . . , 833-864 of the polypeptide or polymerase of SEQ ID NO:21; residues 1-31, 2-32, 3-33, . . . , 853-884 of the polypeptide or polymerase of SEQ ID NO:22; residues 1-31, 2-32, 3-33, . . . , 839-870 of the polypeptide or polymerase of SEQ ID NO:23; residues 1-31, 2-32, 3-33, . . . , 897-928 of the polypeptide or polymerase of SEQ ID NO:24; residues 1-31, 2-32, 3-33, . . . , 929-960 of the polypeptide or polymerase of SEQ ID NO:25.

Nucleic acids may encode fragments which are 33 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 33 amino acids in length such as amino acid residues 1-32, 2-33, 3-34, . . . , 888-920 of the polypeptide or polymerase of SEQ ID NO:14; residues 1-32, 2-33, 3-34, . . . , 857-889 of the polypeptide or polymerase of SEQ ID NO:15; residues 1-32, 2-33, 3-34, . . . , 893-925 of the polypeptide or polymerase of SEQ ID NO:16; residues 1-32, 2-33, 3-34, . . . , 839-871 of the polypeptide or polymerase of SEQ ID NO:17; residues 1-32, 2-33, 3-34, . . . , 839-871 of the polypeptide or polymerase of TaSEQ ID NO:18; residues 1-32, 2-33, 3-34, . . . , 839-871 of the polypeptide or polymerase of SEQ ID NO:19; residues 1-32, 2-33, 3-34, . . . , 868-900 of the polypeptide or polymerase of SEQ ID NO:20; residues 1-32, 2-33, 3-34, . . . , 832-864 of the polypeptide or polymerase of SEQ ID NO:21; residues 1-32, 2-33, 3-34, . . . , 852-884 of the polypeptide or polymerase of SEQ ID NO:22; residues 1-32, 2-33, 3-34, . . . , 838-870 of the polypeptide or polymerase of SEQ ID NO:23; residues 1-32, 2-33, 3-34, . . . , 896-928 of the polypeptide or polymerase of SEQ ID NO:24; residues 1-32, 2-33, 3-34, . . . , 928-960 of the polypeptide or polymerase of SEQ ID NO:25.

Nucleic acids may encode fragments which are 34 amino acids in length, and begin at any amino acid residue of the full length polypeptide or the full length polymerase (e.g., the polypeptides of SEQ ID NOS:14-25 with or without the N-terminal amino acids encoded by the vectors, or the polymerases encoded by the deposited clones). Thus, nucleic acids may encode fragments 34 amino acids in length such as amino acid residues 1-33, 2-34, 3-35, . . . , 887-920 of the polypeptide or polymerase of SEQ ID NO:14; residues 1-33, 2-34, 3-35, . . . , 856-889 of the polypeptide or polymerase of SEQ ID NO:15; residues 1-33, 2-34, 3-35, . . . , 892-925 of the polypeptide or polymerase of SEQ ID NO:16; residues 1-33, 2-34, 3-35, . . . , 838-871 of the polypeptide or polymerase of SEQ ID NO:17; residues 1-33, 2-34, 3-35, . . . , 838-871 of the polypeptide or polymerase of SEQ ID NO:18; residues 1-33, 2-34, 3-35, . . . , 838-871 of the polypeptide or polymerase of SEQ ID NO:19; residues 1-33; 2-34, 3-35, . . . , 867-900 of the polypeptide or polymerase of SEQ ID NO:20; residues 1-33, 2-34, 3-35, . . . , 831-864 of the polypeptide or polymerase of SEQ ID NO:21; residues 1-33, 2-34, 3-35, . . . , 851-884 of the polypeptide or polymerase of SEQ ID NO:22; residues 1-33, 2-34, 3-35, . . . , 837-870 of the polypeptide or polymerase of SEQ ID NO:23; residues 1-33, 2-34, 3-35, . . . , 895-928 of the polypeptide or polymerase of SEQ ID NO:24; residues 1-33, 2-34, 3-35, . . . , 927-960 of the polypeptide or polymerase of SEQ ID NO:25.

Nucleic acids of the invention may encode fragments which contain a continuous series of deleted residues from the amino (N)- or the carboxyl (C)-terminus, or both. For example, any number of amino acids, ranging from 1 to 954, can be deleted from the N-terminus of the encoded fragment. Thus, nucleic acids may encode fragments containing a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, 310 to 320, 320 to 330, 330 to 340, 340 to 350, 350 to 360, 360 to 370, 370 to 380, 380 to 390, 390 to 400, 400 to 410, 410 to 420, 420 to 430, 430 to 440, 440 to 450, 450 to 460, 460 to 470, or 470 to 480 amino acids from the N-terminus of the full length polypeptide or the full length polymerase (e.g., the polypeptides of SEQ ID NOS:14-25 with or without the N-terminal amino acids encoded by the vectors, or the polymerases encoded by the deposited clones).

As another example, nucleic acids of the invention may encode fragments containing a deletion of from 1 to 954 amino acids at the C-terminus. Thus, nucleic acids may encode C-terminal deletion fragments which contain a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, 310 to 320, 320 to 330, 330 to 340, 340 to 350, 350 to 360, 360 to 370, 370 to 380, 380 to 390, 390 to 400, 400 to 410, 410 to 420, 420 to 430, 430 to 440, 440 to 450, 450 to 460, 460 to 470, or 470 to 480 amino acids from the C-terminus of the full length polypeptide or the full length polymerase (e.g., the polypeptides of SEQ ID NOS:14-25 with or without the N-terminal amino acids encoded by the vectors, or the polymerases encoded by the deposited clones).

Furthermore, nucleic acids of the invention may encode fragments which contain combinations of the above N- and C-terminal deletions. Nucleic acids encoding combined N- and C-terminal deletions fragments may encode a fragment containing a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, 310 to 320, 320 to 330, 330 to 340, 340 to 350, 350 to 360, 360 to 370, 370 to 380, 380 to 390, 390 to 400, 400 to 410, 410 to 420, 420 to 430, 430 to 440, 440 to 450, 450 to 460, 460 to 470, or 470 to 480 amino acids from the N-terminus and a deletion of 1 to 10, 10 to 20, 20 to 30, 30 to 40, 40 to 50, 50 to 60, 60 to 70, 70 to 80, 80 to 90, 90 to 100, 100 to 110, 110 to 120, 120 to 130, 130 to 140, 140 to 150, 150 to 160, 160 to 170, 170 to 180, 180 to 190, 190 to 200, 200 to 210, 210 to 220, 220 to 230, 230 to 240, 240 to 250, 250 to 260, 260 to 270, 270 to 280, 280 to 290, 290 to 300, 300 to 310, 310 to 320, 320 to 330, 330 to 340, 340 to 350, 350 to 360, 360 to 370, 370 to 380, 380 to 390, 390 to 400, 400 to 410, 410 to 420, 420 to 430, 430 to 440, 440 to 450, 450 to 460, 460 to 470, or 470 to 480 amino acids from the C-terminus.

Even if deletion of one or more amino acids from the N- and/or C-terminus of an encoded protein results in modification of loss of one or more biological functions of the encoded protein, other functional activities (e.g., enzymatic activities, antigenic activity, immunogenic activity) may still be retained. For example, the ability of shortened polypeptides to induce and/or bind to antibodies which recognize the complete forms of the polypeptides generally will be retained when less than the majority of the residues of the complete or mature polypeptide are removed from the N- and/or C-terminus. Whether a particular encoded polypeptide lacking N- and/or C-terminal residues of a complete polypeptide retains such immunologic activities can readily be determined by routine methods described herein and otherwise known in the art. It is not unlikely that an encoded fragment with a large number of deleted N- and/or C-terminal amino acid residues may retain some antigenic or immunogenic activities. In fact, peptides composed of as few as six amino acid residues may often evoke an immune response, as discussed below.

Nucleic acids may encode fragments which include unique regions, i.e., stretches of amino acids of the polypeptides or polymerases of SEQ ID NOS:14-25 that are less than 100% identical to corresponding stretches of amino acids in other proteins such the polypeptides of SEQ ID NOS:27-34. Unique regions of each encoded polypeptide of the invention are shown in the alignment in Table 35, which indicates the identical and non-identical amino acids of the polymerases of SEQ ID NOS:14-25 (or the polymerases encoded by a deposited clone) as compared to the polypeptides of SEQ ID NOS:27-34. Nucleic acids encoding fragments which contain unique regions are useful for generating highly specific antibodies of the invention, for example by DNA vaccination or by vaccination or screening using recombinant polypeptide. Thus, nucleic acids encoding fragments which contain unique regions are preferred for producing recombinant antigenic fragments of the invention. Additionally, nucleic acids encoding fragments which contain unique regions are especially useful for producing fusion proteins such as proteins produced by DNA shuffling. Using DNA shuffling, nucleic acids encoding fusion proteins are constructed which encode polypeptides comprising fragments from one or more polymerases and which preferably have an enzymatic activity of a polypeptide or polymerase of SEQ ID NOS:14-25 or the polymerases encoded by a deposited clone.

Other nucleic acids encode fragments characterized by structural or functional attributes of the polypeptides of the invention. Such nucleic acids encode fragments which comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson-Wolf program) of full-length polypeptides (e.g., the polypeptides of SEQ ID NOS:14-25). Nucleic acids encoding certain preferred regions include, but are not limited to, those encoding regions of the aforementioned types identified by analysis of the amino acid sequence depicted in SEQ ID NOS:14-25, such preferred regions include; Garnier-Robson predicted alpha-regions, beta-regions, turn-regions, and coil-regions; Chou-Fasman predicted alpha-regions, beta-regions, turn-regions, and coil-regions; Kyte-Doolittle predicted hydrophilic and hydrophobic regions; Eisenberg alpha and beta amphipathic regions; Emini surface-forming regions; and Jameson-Wolf high antigenic index regions, as predicted using the default parameters of these computer programs. These structural or functional attributes can be generated using the various modules and algorithms of the DNA*STAR program set on default parameters.

Among preferred nucleic acids encoding fragments in this regard are those that encode fragments which comprise regions of the polypeptides that combine several structural features, such as several of the features set out above or below.

In another embodiment, nucleic acids may encode polypeptides which comprise or consist of one or more fragments (e.g., regions). For a nucleic acids encoding a polypeptide comprising or consisting of the amino acid sequence of two or more fragments (e.g., regions), the encoded fragments (e.g., regions) may be contiguous with one another. In one embodiment, the encoded fragments (e.g., regions) are not contiguous with one another, i.e., they are separated by one or more amino acid residues.

Preferably, the nucleic acids encode fragments (e.g., regions) which align with the corresponding regions of the full length polypeptide such that they are separated by the same number of amino acid residues as separate them in the full length polypeptide or the full length polymerase (e.g., the polypeptides of SEQ ID NOS:14-25 with or without the N-terminal amino acids encoded by the vectors, (or the polymerases encoded by the deposited clones), or alternatively, the polypeptides of SEQ ID NOS:27-34.

Nucleic acids may encode fragments containing antigenic regions (i.e., regions to which an antibody will bind; epitopes) of the polypeptides of the invention. Nucleic acids may encode antigenic regions as small as 6 amino acids.

The selection of nucleic acids encoding fragments bearing an antigenic region is described above. See, e.g., Sutcliffe, J. G., Shinnick, T. M., Green, N. and Learner, R. A., Science 219:660-666 (1983).

Nucleic acids encoding antigenic fragments preferably encode a sequence of at least seven, more preferably at least nine and most preferably between about 15 to about 30 amino acids. However, nucleic acids may encode a larger portion such as about 30 to about 50 amino acids, or any length up to and including the entire amino acid sequence of a polypeptide of the invention.

In the present invention, nucleic acids may encode antigenic fragments which preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred nucleic acids encoding polypeptides comprising antigenic fragments are at least 10, 15, 20, 25, 30, 35, 40, 45, 50, 55, 60, 65, 70, 75, 80, 85, 90, 95, or 100 amino acid residues in length. Additional non-exclusive preferred nucleic acids which encode antigenic fragments include nucleic acids encoding the fragments disclosed herein, as well as portions thereof. Preferred antigenic fragments include the fragments disclosed herein, as well as any combination of two, three, four, five or more of these fragments.

Polynucleotides comprising nucleic acids encoding one or more antigenic fragments may encode a carrier protein, such as an albumin, either separately or fused in frame the antigenic fragment.

Polynucleotides of the invention may comprise or consist of nucleic acids encoding variants of the full length polypeptide or the fall length polymerase (e.g., the polypeptides of SEQ ID NOS:14-25 with or without the N-terminal amino acids encoded by the vectors, variants of the polypeptides encoded by the deposited clones, and variants of the fragments described above. Encoded variants include polypeptides which are at least 80%, 81%, 82%, 83%, 84%, 85%, 86%, 87%, 88%, 89%, 90%, 91%, 92%, 93%, 94%, 95% 96%, 97%, 98%, or 99% identical to a polypeptide encoded by a deposited clone, to a polypeptide of SEQ ID NOS:14-25, or to a fragment described above.

The invention includes nucleic acids encoding variants which may show a functional activity. Preferably, nucleic acids encode variants which demonstrate a functional activity such as antigenicity or an enzymatic activity described above (e.g., a DNA polymerase activity such as DNA-dependent DNA polymerase activity and/or reverse transriptase activity).

Polynucleotide variants include nucleotide deletions, insertions, inversions, repeats, and substitutions. Polynucleotide variants also include nucleic acids encoding polypeptide deletions, insertions, inversions, repeats, and substitutions (e.g., conservative substitutions, non-conservative substitutions, type substitutions (for example, substituting one hydrophilic residue for another hydrophilic residue, but not a strongly hydrophilic for a strongly hydrophobic, as a rule), primary shifts, primary transpositions, secondary transpositions, and coordinated replacements).

Nucleic acids may encode polypeptide variants in which more than one amino acid (e.g., 2, 3, 4, 5, 6, 7, 8, 9 and 10) is substituted with another amino acid as described above (either conservative or nonconservative). The substituted amino acids can occur in the full length form of the polypeptide, as well as in the fragments described above.

Nucleic acids may encode variants which contain at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of increasing preference, it is preferable for a nucleic acid to encode a variant containing at least one, but not more than 10, 9, 8, 7, 6, 5, 4, 3, 2 or 1 amino acid substitutions. In specific embodiments, the number of additions, substitutions, and/or deletions in the encoded polypeptide (e.g., the full length form and/or fragments described herein), is 1-5, 5-10, 5-25, 5-50, 10-50 or 50-150. Encoded variants may preferably contain conservative amino acid substitutions.

Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. (See Table 41).

Of additional special interest are also substitutions of charged amino acids with another charged amino acid or with neutral amino acids. This may result in proteins with improved characteristics such as less aggregation. Prevention of aggregation is highly desirable. Aggregation of proteins can result in a reduced activity.

Polynucleotides of the invention may be altered by being subjected to random mutagenesis by error-prone PCR, random nucleotide insertion or other methods prior to recombination. Polynucleotides of the invention may be produced by DNA shuffling, gene-shuffling, motif-shuffling, exon-shuffling, and/or codon-shuffling (collectively referred to as “DNA shuffling”). DNA shuffling involves the assembly of two or more DNA segments by homologous or site-specific recombination to generate variation in the polynucleotide sequence. DNA shuffling may be employed to modulate the activities of polypeptides of the invention, such methods can be used to generate polypeptides with altered activity. See, generally, U.S. Pat. Nos. 5,605,793; 5,811,238; 5,830,721; 5,834,252; 5,837,458; and 6,444,468; and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998). Polynucleotides of the invention encode contain one or more components, motifs, sections, parts, domains, fragments, etc., of a polypeptide of the invention joined to one or more components, motifs, sections, parts, domains, fragments, etc. of one or more heterologous molecules, preferably the polymerases in SEQ ID NOS:14-25.

Nucleic acids encoding fragments, mutants, variants, or full length polypeptides of the invention may be “free-standing,” or comprised within a larger polynucleotide of which the nucleic acid encoding the fragment, mutant, variant, or full length polypeptide forms a part or region.

Thus, polynucleotides may encode one or more additional amino acids and/or one or more heterologous sequences such as those described herein. For instance, polynucleotides may comprise a codon for methionine added to the 5′ end of the nucleic acid encoding the polypeptide, such that the encoded polypeptide comprises a Met residue at the N-terminus, thus allowing for recombinant expression. Also, the polynucleotide may comprise a nucleic acid encoding additional a sequence of amino acids, particularly charged amino acids, which may fused to the N-terminus of the encoded polypeptide to improve stability and persistence, in the host cell, during purification, or during subsequent handling and storage. A preferred polynucleotide encodes a fusion protein comprising a heterologous region from immunoglobulin that is useful to solubilize proteins.

Thus, polynucleotides may comprise the nucleic acids above and may also encode one or more additional amino acids and/or one or more heterologous polypeptides. Heterologous polypeptides include secretory or leader sequences, pro-sequences, tags or other sequences which aid in purification, such as multiple histidine residues, or an additional sequence for stability during recombinant production.

Preferably, polynucleotides encode polypeptides which demonstrate a functional activity such as an enzymatic activity described above (e.g., a DNA polymerase activity such as DNA-dependent DNA polymerase activity and/or reverse transriptase activity) or antigenicity.

As indicated, nucleic acid molecules of the present invention which encode a polypeptide of the invention may include, but are not limited to those encoding the amino acid sequence of the polypeptide (e.g., full length, fragment, mutant, or variant) by itself; the coding sequence for the polypeptide and additional sequences, such as those encoding the leader or secretory sequence, such as a pre-, or pro- or prepro-protein sequence; the coding sequence of the polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals for eucaryotic expression, for example—ribosome binding and stability of mRNA; an additional coding sequence which codes for additional amino acids, such as heterologous sequences, for example those which provide additional functionalities. Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984). As discussed below, other such nucleic acids encoding fusion proteins include those encoding a polypeptide of the invention fused to Fc at the N- or C-terminus.

### 3. Cloning and Expression of the Polypeptides of the Invention.

Organisms from which to clone polypeptides of the invention (e.g., thermophilic eubacteria) can be isolated from many sources, for example, a compost pile. Suitable organisms include, but are not limited to, archaeabacteria and eubacteria. Nucleic acids encoding polypeptides of the invention may be cloned from eubacteria from one or more of the genera Acanthamoeba, Acinetobacter, Actinomyces, Actinomyces, Agrobacterium, Anisakids, Ascaris, Aspergillus, Azomonas, Azotobacter, Babesia, Bacillus, Bacteroides, Balantidium, Bdellovibrio, Bifidobacterium, Bordetella, Borrelia, Bradyrhizobium, Brucella, Caldibacillus, Caldicellulosiruptor, Campylobacter, Candida, Ceratocystis, Chlamydia, Chlorobium, Chloroflexus, Chromatium, Citrobacter, Clostridium, Corynebacterium, Coxiella, Cryphonectria, Cryptosporidium, Dictyoglomus, Echinococcus, Etamoeba, Enterobacter, Enterobius, Enterococcus, Escherichia, Francisella, Fusobacterium, Gambierdiscus, Gardnerella, Gelidium, Giardia, Haloarcula, Halobacterium, Helicobacter, Haemophilus, Isospora, Klebsiella, LactoBacillus, Legionella, Leptospira, Listeria, Moraxella, Mucor, Mycobacterium, Mycoplasma, Naegleria, Neisseria, Necator, Nocardia, Nosema, Paragonimus, Pasteurella, Penicillium, Phytophthora, Pityrosporum, Plasmodium, Pneumocystis, Propionibacterium, Proteus, Pseudomonas, Rhizopus, Rickettsia, Rhizobium, Rhodopseudomonas, Saccharomyces, Salmonella, Schizosaccharomyces, Serratia, Shigella, Schistosoma, Staphylococcus, Stella, Streptococcus, Taenia, Thermatoga, Thermus, Toxoplasmosis, Treponema, Trichinella, Trichomonas, Tripanosoma, Veillonella, Vibrio, Yersinia and used in the practice of the present invention. Nucleic acids encoding polypeptides of the invention may be cloned from archaeabacteria from one or more of the genera Pyrodictium, Thermoproteus, Thermococcus, Methanococcus, Methanobacterium, Methanomicrobium, and Halobacterium.

In some embodiments, a nucleic acid encoding a polypeptide of the invention may be cloned from a suitable organism including, but not limited to, those listed above. In some embodiments, a nucleic acid encoding such a polypeptide may be cloned from one or more eubacteria including, but not limited to, Clostridium spp. (e.g., Clostridium stercorarium, Clostridium thermosulfurogenes, etc.), Caldibacillus spp. (e.g., Caldibacillus cellulovorans CompA.2), Caldicellulosiruptor spp. (e.g., Caldicellulosiruptor Tok13B, Caldicellulosiruptor Tok7B, Caldicellulosiruptor RT69B), Bacillus spp. (e.g., Bacillus caldolyticus EA1), Thermus spp. (e.g., Thermus RT41A), Dictyoglomus spp. (e.g., Dictyoglomus thermophilum), Spirochaete spp., and Tepidomonas spp.

Clostridium stercorarium was obtained from Watkato University. Clostridium stercorarium (isolated from compost) is available as ATCC 35414. Another suitable source from which to isolate a gene coding for a polypeptide of the present invention is Clostridium thermosulfurogenes. Clostridium thermosulfurogenes was obtained from a thermal spring in Yellowstone Notional Park, USA and is available as ATCC 33743. Other similar organisms can be isolated from thermal environments or can be obtained from various depositories.

To clone a gene encoding a polypeptide of the invention, for example, a eubacterial DNA polymerase, isolated DNA that encodes the polymerase is obtained from bacterial cells using standard techniques and may be used to construct a recombinant DNA library in a vector. Any vector can be used to clone wild type or mutant polypeptides of the present invention. However, the vector used is preferably compatible with the host in which the recombinant DNA library will be transformed.

Prokaryotic vectors for constructing a library include plasmids such as those capable of replication in E. coli, for example, pBR322, ColE1, pSC101, pUC-vectors (pUC18, pUC19, etc.: In: Molecular Cloning, A Laboratory Manual, Cold Spring Harbor Laboratory Press, Cold Spring Harbor, N.Y. (1982); and Sambrook, et al., In: Molecular Cloning A Laboratory Manual (2d ed.) Cold Spring Harbor Laboratory Press, Cold Spring Harbor, N.Y. (1989)). Bacillus plasmids include pC194, pC221, pC217, etc. Such plasmids are disclosed by Glyczan, T. in: The Molecular Biology Bacilli, Academic Press, York (1982), 307-329. Suitable Streptomyces plasmids include pIJ101 (Kendall, et al, J. Bacteriol 169:4177-4183 (1987)). Pseudomonas plasmids are reviewed by John, et al, (Rad. Insec. Dis. 8:693-704 (1986)), and Igaki, (Jpn. J. Bacteriol. 33:729-742 (1978)). Broad-host range plasmids or cosmids, such as pCP13 (Darzins and Chakrabarbary, J Bacteriol. 159:9-18, 1984) can also be used for the present invention. Preferred vectors for cloning the genes of the present invention are prokaryotic vectors. For example pET and pUC vectors can be used to clone genes of the present invention.

A preferred host for cloning wild type or mutant DNA polymerase genes of the invention is a prokaryotic host. A preferred prokaryotic host is E. coli. However, wild type or mutant DNA polymerase genes of the present invention may be cloned in other prokaryotic hosts including, but not limited to, Escherichia, Bacillus, Streptomyces, Pseudomonas, Salmonella, Serratia, and Proteus. Bacterial hosts of particular interest include E. coli BL21SI, which may be obtained from Invitrogen Corporation, Carlsbad, Calif.

Eukaryotic hosts for cloning and expression of wild type or mutant DNA polymerases of the present invention include yeast, fungi, insect and mammalian cells. Expression of the desired DNA polymerase in such eukaryotic cells may require the use of eukaryotic regulatory regions which include eukaryotic promoters. Cloning and expressing wild type or mutant genes encoding polypeptides of the invention in eukaryotic cells may be accomplished by known techniques using known eukaryotic vector systems.

Once a DNA library has been constructed in a particular vector, an appropriate host can be transformed by one of many well-known techniques and transformed host cells may be screened for a desired activity. For example transformed colonies may be plated at a density of approximately 200-300 colonies per petri dish. Colonies can then be screened for expression of a heat stable DNA polymerase by transferring transformed colonies to nitrocellulose membranes. After the transferred cells are grown on the membranes (approximately 12 hours), the cells are lysed by standard techniques, and the membranes are then treated at 95° C. for 5 minutes to inactivate the endogenous E. coli enzyme. Other procedures can be used, for example, other temperatures may be used to inactivate host polymerases depending on the host used and the temperature stability of the DNA polymerase to be cloned. Stable DNA polymerase activity can then be detected by assaying for the presence of DNA polymerase activity using any of the well known techniques. See e.g., Sanger, et al., Gene 97:119-123 (1991), which is hereby incorporated by reference in its entirety. A gene encoding a DNA polymerase of the present invention can be cloned for example by using the procedure described by Sagner, et al., supra.

Recombinant hosts, each containing a nucleic acid encoding a polypeptide of the invention, have been made. The genes encoding Clostridium stercorarium, Clostridium thermosulfurogenes, Caldibacillus cellulovorans CompA.2, Caldicellulosiruptor Tok 13B.1, Caldicellulosiruptor Tok7B.1, Caldicellulosiruptor Rt69B.1, Bacillus caldolyticus EA1, Thermus Rt41A.1, Dictyoglomus thermophilum, Caldicellulosiruptor saccharolyticus, Spirochaete, and Tepidomonas DNA polymerases have been used to generate recombinant E. coli BL21SI using the vector pET26B. The genes have also been cloned and sequenced and the DNA sequences are represented in SEQ ID NOS: 2-13. The corresponding amino acid sequences are represented in SEQ ID NOS:14-25. The genes can be inserted into other plasmids and/or hosts for expression.

### 4. Enhancing Expression of the Polypeptides of the Invention.

To optimize expression of a wild type or mutant polypeptide of the present invention, a nucleic acid sequence encoding the polypeptide may be operatively linked to a promoter, for example, an inducible or constitutive promoter. Suitable promoters are well known to those skilled in the art and may be selected to express high levels of a polypeptide in a recombinant host. Similarly, high copy number vectors, well known in the art, may be used to achieve high levels of expression. Inducible, highly active promoters may be used in conjunction with high copy number vectors to enhance expression of a polypeptide of the invention in a recombinant host.

To express a polypeptide in a prokaryotic cell (such as, E. coli, B. subtilis, Pseudomonas, etc.), it is preferred to operably link a nucleic acid sequence encoding the polypeptide to a functional prokaryotic promoter. However, the promoter associated with the coding sequence in its native host may function in prokaryotic hosts allowing expression of the polypeptide of the invention. Thus, natural thermophilic eubacterial promoters (e.g., from Clostridium spp., Caldibacillus spp., Caldicellulosiruptor spp., Bacillus spp., Thermus spp., Dictyoglomus spp., etc.) promoters or other promoters may be used to express the polypeptides of the invention. Such other promoters may be used to enhance expression and may either be constitutive or regulatable (i.e., inducible or derepressible) promoters. Examples of constitutive promoters include the int promoter of bacteriophage λ, and the bla promoter of the β-lactamase gene of pBR322. Examples of inducible prokaryotic promoters include the major right and left promoters of bacteriophage λ (P_{R} and P_{L}), trp, recA, lacZ, lacI, tet, gal, trc, and tac promoters of E. coli. The B. subtilis promoters include α-amylase (Ulmanen, et al., J. Bacteriol. 162:176-182 (1985)) and Bacillus bacteriophage promoters (Gryczan, T., In: The Molecular Biology Of Bacilli, Academic Press, New York (1982)). Streptomyces promoters are described by Ward, et al., Mol. Gen. Genet. 203:468-478 (1986)). Prokaryotic promoters are also reviewed by Glick, J. Ind. Microbiol. 1:277-282 (1987); Cenatiempto, Y., Biochimie 68:505-516 (1986); and Gottesman, Ann. Rev. Genet. 18:415-442 (1984). Generally presence of a ribosomal binding site upstream of the gene-encoding sequence is preferred. Such ribosomal binding sites are disclosed, for example, by Gold, et al., Ann. Rev. Microbiol. 35:365-404 (1981).

To enhance expression of a polypeptide of the invention in a eukaryotic cell, many well known eukaryotic promoters and hosts may be used. Preferably, however, enhanced expression of a polypeptide of the invention is accomplished in a prokaryotic host. A preferred prokaryotic host for overexpressing this enzyme is E. coli.

### 5. Isolation and Purification of the Polypeptides of the Invention.

Polypeptides of the present invention (e.g., DNA polymerases from thermophilic eubacteria, and fragments and mutants thereof) are preferably produced by fermentation of a recombinant host containing and expressing a cloned polypeptide gene. However, wild type and mutant DNA polymerases of the present invention may be isolated from any organism (e.g., a thermophilic eubacterial strain) that produces a polypeptide of the present invention. Fragments of the polypeptides of the invention are also included in the present invention. Such fragments include proteolytic fragments, deletion fragments and especially fragments having polymerase activity. Preferred fragments include those having an RNA-directed DNA polymerase activity and, optionally, lacking one or more exonuclease activity found in the wild type polypeptide.

Any nutrient that can be assimilated by a cell or organism naturally expressing a polypeptide of the invention or by a host containing a cloned nucleic acid sequence encoding a polypeptide of the invention may be present in the culture medium. Culture conditions should be selected case by case according to the strain used and the composition of the culture medium. Such selection is routinely practiced by those skilled in the art. Antibiotics may also be added to the media to insure maintenance of vector DNA containing the desired gene to be expressed. Media formulations are described for example in DSM or ATCC Catalogs and Sambrook, et al., In: Molecular Cloning, a Laboratory Manual (2nd ed.), Cold Spring Harbor Laboratory Press, Cold Spring Harbor, N.Y. (1989).

Cells or organisms naturally expressing the polypeptides of the invention and/or recombinant host cells producing the polypeptide of the invention can be separated from liquid culture, for example, by centrifugation. In general, the collected cells are dispersed in a suitable buffer, and then broken down by ultrasonic treatment, chemical treatment or by other well known procedures to allow extraction of the enzymes by the buffer solution. After removal of cell debris by ultracentrifugation or centrifugation, the polypeptide can be purified by standard protein purification techniques such as extraction, precipitation, chromatography, affinity chromatography, electrophoresis or the like. Assays to detect presence of DNA polymerase during purification are well known in the art and can be used during and/or after conventional biochemical purification methods to determine the presence of these enzymes.

### 6. Uses of the Polypeptides of the Invention.

Wild type and mutant polypeptides of the present invention may be used to prepare cDNA from RNA templates including mRNA, tRNA, rRNA, nuclear RNA, and total RNA isolated from a sample. Polymerases of the present invention may be used in a method for reverse transcribing RNA into complementary DNA (cDNA) and amplifying the cDNA, comprising:

(a) providing a first and second primer, wherein the first primer is sufficiently complementary to a target RNA to hybridize therewith;

(b) hybridizing the first primer to the RNA molecule in the presence of a DNA polymerase of the invention, under conditions such that a cDNA molecule complementary to the target RNA is synthesized;

(c) treating the reaction mixture to provide single stranded cDNA;

(d) hybridizing the second primer to the cDNA molecule in the presence of a DNA polymerase of the invention, under conditions such that an extension product is synthesized to provide a double-stranded cDNA molecule; and, optionally,

(e) amplifying the double-stranded cDNA molecule of (d) (e.g., by a polymerase chain reaction). Amplification may be performed using a polypeptide of the invention and/or an additional polymerase. Suitable additional polymerases, preferably from thermophilic organisms, are known in the art (e.g., Taq DNA polymerase, Pfu DNA polymerase, Tne DNA polymerase, etc.). Methods of reverse transcribing an RNA may be performed in buffers comprising Mg²⁺, which buffers may or may not, and preferably do not, comprise Mn²⁺. Suitable conditions may also comprise the addition of one or more nucleotides, one or more of which may be modified (e.g., may comprise a label such as a fluorescent label and/or a reactive functional group to which a label may be attached).

The invention also relates to a method of preparing cDNA from messenger RNA (mRNA), comprising:

(a) contacting RNA with an oligo(dT) primer or other complementary primer to form a complex, and

(b) contacting the complex formed in step (a) with the polypeptide or mutant of the invention and dNTPs, whereby a cDNA-RNA hybrid is obtained. Methods of preparing a cDNA from an mRNA may be performed in buffers comprising Mg²⁺, which buffers may or may not, and preferably do not, comprise Mn²⁺.

If the reaction mixture in step (b) further comprises an appropriate oligonucleotide that is complementary to the cDNA being produced, it is also possible to obtain dsDNA following first strand synthesis. Thus, the invention is also directed to a method of preparing dsDNA with the polypeptides, fragments and/or mutants thereof of the present invention.

A thermostable DNA polymerase for use in amplifying the dsDNA can be used with the polypeptides of the present invention in a coupled reverse transcription/amplification reaction. The same reaction buffer solution can be used for both enzymes thereby replacing prior methods requiring the need to change, adjust or dilute the buffer components including divalent cations, salts, and pH between the reverse transcription and amplification steps.

DNA polymerases (including thermostable DNA polymerases) that may be used in combination with the polypeptides of the present invention include, but are not limited to, Taq DNA polymerase, Tne DNA polymerase, Tma DNA polymerase, Pfu DNA polymerase, Tfl DNA polymerase, Tth DNA polymerase, Thr DNA polymerase, Pwo DNA polymerase, Bst DNA polymerase, Bca DNA polymerase, VENT DNA polymerase, T7 DNA polymerase, T5 DNA polymerase, DNA polymerase III, Klenow fragment DNA polymerase, Stoffel fragment DNA polymerase, and mutants, fragments or derivatives thereof.

The present invention is suitable for reverse transcribing and amplifying RNA from a number of sources. The RNA template may be contained within a nucleic acid preparation from an organism. Examples of organisms from which RNA may be prepared include, but are not limited to, animals, plants, yeast, viruses, and/or bacteria. The preparation may contain cell debris and other components, crude or purified total RNA, or crude or purified mRNA. The RNA template may be a population of heterogeneous RNA molecules in a sample or a specific target RNA molecule. The RNA may be produced in a cell or using a cell free system. RNA from any source can be used in the present invention.

RNA suitable for use in the present methods may be contained in any source that comprises RNA, for example in a biological sample hypothesized to contain a specific target RNA. The biological sample may be a heterogeneous sample in which RNA is a small portion of the sample, as in for example, a blood sample or a patient tissue sample, for example, one obtained by a biopsy. Thus, the method is useful for clinical detection and diagnosis. The RNA target may be indicative of a specific disease or infectious agent.

The wild type and mutant polypeptides of the present invention may be used in well known assays such as DNA sequencing, DNA labeling, DNA amplification and cDNA synthesis reactions. For example, eubacterial DNA polymerase mutants devoid of or substantially reduced in 5′-to-3′ exonuclease activity, or containing one or mutations in the O-helix that make the enzyme nondiscriminatory for dNTPs and ddNTPs (e.g., a Phe754-to-Tyr754 mutation of SEQ ID NO:2) are especially useful for DNA sequencing, DNA labeling, and DNA amplification reactions and cDNA synthesis.

Moreover, mutants containing two or more of these properties are also especially useful for DNA sequencing, DNA labeling, DNA amplification or cDNA synthesis reactions. As is well known, sequencing reactions (isothermal DNA sequencing and cycle sequencing of DNA) require the use of DNA polymerases. Dideoxy-mediated sequencing involves the use of a chain-termination technique which uses a specific polymer for extension by DNA polymerase, a base-specific chain terminator and the use of polyacrylamide gels to separate the newly synthesized chain-terminated DNA molecules by size so that at least a part of the nucleotide sequence of the original DNA molecule can be determined. Specifically, a DNA molecule is sequenced by using four separate DNA sequence reactions, each of which contains different base-specific terminators. For example, the first reaction may contain a G-specific terminator, the second reaction may contain a T-specific terminator, the third reaction may contain an A-specific terminator, and a fourth reaction may contain a C-specific terminator. Preferred terminator nucleotides include dideoxyribonucleoside triphosphates (ddNTPs) such as ddATP, ddTTP, ddGTP, ddITP and ddCTP. Analogs of dideoxyribonucleoside triphosphates may also be used and are well known in the art.

When forming a DNA molecule, ddNTPs lack a hydroxyl residue at the 3′ position of the ribose ring and thus, although they can be incorporated by DNA polymerases into the growing DNA chain, the absence of the 3′-hydroxy residue prevents formation of the next phosphodiester bond resulting in termination of extension of the DNA molecule. Thus, when a small amount of one ddNTP is included in a sequencing reaction mixture, there is competition between extension of the chain and base-specific termination resulting in a population of synthesized DNA molecules which are shorter in length than the DNA template to be sequenced. By using four different ddNTPs in one or more enzymatic reactions, populations of the synthesized DNA molecules can be separated by size so that at least a part of the nucleotide sequence of the original DNA molecule can be determined. DNA sequencing by dideoxy-nucleotides is well known and is described by Sambrook, et al., In: Molecular Cloning, a Laboratory Manual, Cold Spring Harbor Laboratory Press, Cold Spring Harbor, N.Y. (1989). Sequencing apparatuses based on dideoxy termination are commercially available. Other sequencing protocols, e.g., using fluorescent dyes, are known in the art and are also suitable for use with the present invention. As will be readily recognized, the polypeptides and mutants thereof of the present invention may be used in such sequencing reactions.

As is well known, detectably labeled nucleotides are typically included in sequencing reactions. Any number of labeled nucleotides can be used in sequencing (or labeling) reactions, including, but not limited to, radioactive isotopes, fluorescent labels, chemiluminescent labels, bioluminescent labels, and enzyme labels. Wild type and mutant polypeptides of the present invention may be useful for incorporating [α-S] nucleotides ([α-S]dATP, [α-S]dTTP, [α-S]dCTP and [α-S]dGTP) during sequencing (or labeling) reactions. Thus, the polypeptides of the present invention are particularly suited for sequencing or labeling DNA molecules with [α-³⁵S]dNTPs.

Polymerase chain reaction (PCR), a well known DNA amplification technique, is a process by which DNA polymerase and deoxyribonucleoside triphosphates are used to amplify a target DNA template. In PCR reactions, two primers, one complementary to the 3′ termini (or near the 3′-terminus) of the first strand of the DNA molecule to be amplified, and a second primer complementary to the 3′ termini (or near the 3′-terminus) of the second strand of the DNA molecule to be amplified, are hybridized to their respective DNA strands. After hybridization, DNA polymerase, in the presence of deoxyribonucleoside triphosphates, allows synthesis of a third DNA molecule complementary to the first strand and a fourth DNA molecule complementary to the second strand of the DNA molecule to be amplified. This synthesis results in two double stranded DNA molecules. Such double stranded DNA molecules may then be used to provide DNA templates for synthesis of additional DNA molecules by providing a DNA polymerase, primers, and deoxyribonucleoside triphosphates. As is well known, the additional synthesis is carried out by “cycling” the original reaction (with excess primers and deoxyribonucleoside triphosphates) allowing multiple denaturing and synthesis steps. Typically, denaturing of double stranded DNA molecules to form single stranded DNA templates is accomplished by high temperatures. DNA polymerases of the present invention may be heat stable DNA polymerases at higher temperatures if appropriate mutations are introduced, and thus will survive such thermal cycling during DNA amplification reactions and would then be suited for PCR reactions, particularly where high temperatures are used to denature the DNA molecules during amplification.

7. Antibodies that Specifically Bind the Polypeptides of the Invention

The present invention concerns the production and use of molecules (polypeptides and antibodies) that are capable of “specific binding” to one another. As used herein, a molecule is said to be capable of “specific binding” to another molecule, if such binding is dependent upon the respective structures of the molecules. The known capacity of an antibody to bind to an antigen is an example of “specific binding.” Such interactions are in contrast to non-specific binding between classes of compounds, irrespective of their chemical structure (such as the binding of proteins to nitrocellulose, etc.). Most preferably, the antibodies of the present invention exhibit “highly specific binding,” such that they will be incapable or substantially incapable of binding to closely related polypeptides (e.g., the polymerases of SEQ ID NOS:27-34). Indeed, preferred antibodies of the present invention exhibit the capacity to bind to a polypeptide of SEQ ID NOS:14-25 or a polypeptide encoded by a deposited clone, but are substantially incapable of binding the polymerases of SEQ ID NOS:27-34; such antibodies are capable of highly specific binding to a polypeptide of SEQ ID NOS:14-25 or a polypeptide encoded by a deposited clone, as that phrase is used herein. In preferred embodiments, antibodies of the invention do not include antibodies that bind to the polymerases of SEQ ID NOS:27-34.

However, it is immediately apparent to one of ordinary skill that even antibodies that bind to other proteins, i.e., which are cross-reactive because they recognize an epitope (antigenic region) shared between a polypeptide of the invention and another polypeptide, are still useful for “hot start” of methods of the invention. The present invention further relates to antibodies and T-cell antigen receptors (TCR) which specifically bind the polypeptides of the present invention. Antibodies may be polyclonal and/or monoclonal. They may be prepared against an entire polypeptide or against a fragment of the polypeptide.

The present invention concerns the production and use of molecules (polypeptides and antibodies) that are capable of “specific binding” to one another. As used herein, a molecule is said to be capable of “specific binding” to another molecule, if such binding is dependent upon the respective structures of the molecules. The known capacity of an antibody to bind to an antigen is an example of “specific binding.” Such interactions are in contrast to non-specific binding between classes of compounds, irrespective of their chemical structure (such as the binding of proteins to nitrocellulose, etc.). Most preferably, the antibodies of the present invention exhibit “highly specific binding,” such that they will be incapable or substantially incapable of binding to closely related polypeptides (e.g., the polymerases of SEQ ID NOS:27-34). Indeed, preferred antibodies of the present invention exhibit the capacity to bind to a polypeptide of SEQ ID NOS:14-25 or a polypeptide encoded by a deposited clone, but are substantially incapable of binding the polymerases of SEQ ID NOS:27-34; such antibodies are capable of highly specific binding to a polypeptide of SEQ ID NOS:14-25 or a polypeptide encoded by a deposited clone, as that phrase is used herein. In preferred embodiments, antibodies of the invention do not include antibodies that bind to the polymerases of SEQ ID NOS:27-34.

However, it is immediately apparent to one of ordinary skill that even antibodies that bind to other proteins, i.e., which are cross-reactive because they recognize an epitope (antigenic region) shared between a polypeptide of the invention and another polypeptide, are still useful for “hot start” of methods of the invention.

The antibodies of the present invention include IgG (including IgG1, IgG2, IgG3, and IgG4), IgA (including IgA1 and IgA2), IgD, IgE, IgM, and IgY. As used herein, the term “antibody” (Ab) is meant to include whole antibodies, including single-chain whole antibodies, and antigen-binding fragments thereof. In some embodiments, antigen-binding fragments may be mammalian antigen-binding antibody fragments that include, but are not limited to, Fab, Fab′ and F(ab′)2, Fd, single-chain Fvs (scFv), single-chain antibodies, disulfide-linked Fvs (sdFv) and fragments comprising either a VL or VH domain.

Antibodies of the invention may be prepared from any animal origin including birds and mammals. Preferably, the antibodies prepared from mammals, (e.g., human, murine, rabbit, goat, guinea pig, camel, or horse). Other preferred sources may be avian (e.g., chicken).

Antibodies may be used for the detection of the polypeptides in an immunoassay, such as ELISA, Western blot, radioimmunoassay, enzyme immunoassay, and may be used in immunocytochemistry. In some embodiments, an anti-polypeptide antibody may be in solution and the polypeptide to be recognized may be in solution (e.g., an immunopreciptitation) or may be on or attached to a solid surface (e.g., a Western blot). In other embodiments, the antibody may be attached to a solid surface and the polypeptide may be in solution (e.g., affinity chromatography).

Antibodies to the polypeptides of the invention may be used to determine the presence, absence or amount of one or more of the polypeptides in a sample. The amount of specifically bound polypeptide may be determined using an antibody to which is attached a label or other marker, such as a radioactive, a fluorescent, or an enzymatic label. Alternatively, a labeled secondary antibody (e.g., an antibody that recognizes the antibody that is specific to the polypeptide) may be used to detect a polypeptide-antibody complex between the specific antibody and the polypeptide.

Antibodies of the invention may be used to modulate one or more activities of the polypeptides of the invention. For example, a polypeptide of the invention may be contacted with an antibody under conditions such that the antibody binds to the polypeptide. A polypeptide bound by antibody may have the same or different activities as the same polypeptide unbound. In some embodiments, a polypeptide of the invention bound by an antibody of the invention may have a reduced, substantially reduced or eliminated enzymatic activity while bound. For example, a bound polypeptide may display no detectable RNA-dependent and/or DNA-dependent DNA polymerase activity. Preferably, the activity is recovered when the antibody is no longer bound. Thus, in the previous example, RNA-dependent and/or DNA-dependent DNA polymerase activity may be recovered when the polypeptide is no longer bound by the antibody. In some embodiments, antibodies of the present invention may bind to a polypeptide of the invention under some conditions (e.g., temperature, ionic strength, etc.) and may not bind under other conditions (e.g., at an elevated temperature).

One or more of the polypeptides of the invention may be used as immunogens to prepare polyclonal an/or monoclonal antibodies capable of binding the polypeptides using techniques well known in the art (Harlow & Lane, Antibodies: A Laboratory Manual, Cold Spring Harbor Laboratory, Cold Spring Harbor, N. Y., 1988). In brief, antibodies are prepared by immunization of suitable subjects (e.g., mice, rats, rabbits, goats, etc.) with all or a part of the polypeptides of the invention. If the polypeptide or fragment thereof is sufficiently immunogenic, it may be used to immunize the subject. If necessary or desired to increase immunogenicity, the polypeptide or fragment may be conjugated to a suitable carrier molecule (e.g., BSA, KLH, and the like). Polypeptides of the invention or fragments thereof may be conjugated to carriers using techniques well known in the art. For example, they may be directly conjugated to a carrier using, for example, carbodiimide reagents. Other suitable linking reagents are commercially available from, for example, Pierce Chemical Co., Rockford, Ill.

Suitably prepared polypeptides of the invention or fragments thereof may be administered by injection over a suitable time period. They may be administered with or without the use of an adjuvant (e.g., Freunds). They may be administered one or more times until antibody titers reach a desired level.

In some embodiments, it may be desirable to produce monoclonal antibodies to the polypeptides of the invention or fragments thereof. Monoclonal antibodies can be prepared from the immune cells of animals (e.g., mice, rats, etc.) immunized with all or a portion of one or more polypeptide of the invention using conventional procedures, such as those described by Kohler and Milstein, Nature, 256, pp. 495-497 (1975). Hybridoma cell lines may be prepared by isolating antibody secreting cells of the host animal from lymphoid tissue (such as the spleen) and fusing them with mouse myeloma cells (for example, SP2/0-Ag14 murine myeloma cells) in the presence of polyethylene glycol. The fused cells may be diluted into selective media and plated in multiwell tissue culture dishes. The hybridoma cells which secrete the desired antibodies can then be identified testing the supernatants for antibodies of the desired specificity using standard techniques (e.g., ELISA, etc.). The resultant hybridoma cells can be grown in static culture, hollow fiber bioreactors or used to produce ascitic tumors in mice in order to produce the monoclonal antibodies. Thus, the present invention provides monoclonal antibodies specific to the polypeptides of the invention, as well as cell lines producing such monoclonal antibodies.

In some embodiments, it may be desirable to use a fragment of an antibody that is capable of binding a polypeptide of the invention or fragment thereof. For example, Fab, Fab′, of F(ab′)₂ fragments may be produced using techniques well known in the art.

In some embodiments, the present invention contemplates a composition comprising a polypeptide of the invention and an antibody to the polypeptide of the invention. In such a composition, the antibody may be bound to the polypeptide under one set of conditions (e.g., temperature, ionic strength, etc.) and may dissociate from the polypeptide under other conditions (e.g., at an increased temperature).

### 8. Reverse Transcriptase Enzymes for Use in the Invention

Enzymes for use in compositions, methods and kits of the invention include any enzyme having reverse transcriptase activity. Such enzymes include, but are not limited to, retroviral reverse transcriptase, retrotransposon reverse transcriptase, hepatitis B reverse transcriptase, cauliflower mosaic virus reverse transcriptase, bacterial reverse transcriptase, Tth DNA polymerase, Taq DNA polymerase (Saiki, R. K., et al., Science 239:487-491 (1988); U.S. Pat. Nos. 4,889,818 and 4,965,188), Tne DNA polymerase (PCT Publication No. WO 96/10640), Tma DNA polymerase (U.S. Pat. No. 5,374,553) and mutants, fragments, variants or derivatives thereof (see, e.g., commonly owned U.S. Pat. Nos. 5,948,614 and 6,015,668, which are incorporated by reference herein in their entireties). Preferably, reverse transcriptases for use in the invention include retroviral reverse transcriptases such as M-MLV reverse transcriptase, AMV reverse transcriptase, RSV reverse transcriptase, RAV reverse transcriptase, MAV reverse transcriptase, and generally ASLV reverse transcriptases. As will be understood by one of ordinary skill in the art, modified reverse transcriptases may be obtained by recombinant or genetic engineering techniques that are routine and well-known in the art. Mutant reverse transcriptases can, for example, be obtained by mutating the gene or genes encoding the reverse transcriptase of interest by site-directed or random mutagenesis. Such mutations may include point mutations, deletion mutations and insertional mutations. For example, one or more point mutations (e.g., substitution of one or more amino acids with one or more different amino acids) may be used to construct mutant reverse transcriptases for use in the present invention.

Preferred enzymes for use in the invention include those that are reduced, substantially reduced, or lacking in RNase H activity. Such enzymes that are reduced or substantially reduced in RNase H activity may be obtained by mutating, for example, the RNase H domain within the reverse transcriptase of interest, for example, by introducing one or more (e.g., one, two, three, four, five, ten, twelve, fifteen, twenty, thirty, etc.) point mutations, one or more (e.g., one, two, three, four, five, ten, twelve, fifteen, twenty, thirty, etc.) deletion mutations, and/or one or more (e.g., one, two, three, four, five, ten, twelve, fifteen, twenty, thirty, etc.) insertion mutations as described above. In some embodiments, the reverse transcriptase of the invention does not contain a modification or mutation in the RNase H domain and preferably does not contain a modification which reduces RNase H activity. In one aspect, the reverse transcriptase of the invention has 90%, 95%, or 100% of the RNase H activity compared to the corresponding wildtype reverse transcriptase.

### 9. Kits

The wild type and mutant polypeptides of the invention are suited for the preparation of a kit. Kits comprising wild type or mutant polypeptides may be configured for use in any procedure known to those skilled in the art. Suitable kits may be prepared for, for example, cDNA synthesis and/or amplification, detectably labeling DNA molecules, and DNA sequencing. See U.S. Pat. Nos. 4,962,020, 5,173,411, 4,795,699, 5,498,523, 5,405,776 and 5,244,797. Such kits may comprise a carrier that may be compartmentalized to receive in close confinement one or more containers such as vials, test tubes, wells, solid supports, chips and the like. Preferably at least one of such containers contains components or a mixture of components needed to perform DNA sequencing, DNA labeling, DNA amplification, or cDNA synthesis.

A kit for sequencing DNA may comprise a number of containers each of which may contain one or more components. A first container may, for example, contain a substantially purified sample of a polypeptide of the invention, for example, a DNA polymerase from a thermophilic eubacterium, fragment or mutant thereof. A second container may contain one or a number of types of nucleotides needed to synthesize a DNA molecule complementary to a nucleic acid template. A third container may contain one or a number of different types of dideoxynucleoside triphosphates, optionally labeled with one or more detectable groups. A fourth container may contain pyrophosphatase. In addition to the above containers, additional containers may be included in the kit that contain other components for carrying out a desired procedure, for example, one or a number of DNA primers (e.g., oligo(dT) primers), optionally such primers may be labeled.

A kit used for amplifying DNA may comprise, for example, a first container containing a substantially or essentially pure preparation of mutant or wild type polypeptide of the invention, for example, a DNA polymerase from a thermophilic eubacterium, and one or a number of additional containers that contain a single type of nucleotide or mixtures of nucleotides. Various primers may or may not be included in a kit for amplifying DNA. In some embodiments, the polypeptides of the invention may be used in a mixture with one or more polypeptides having one or more enzymatic activities (e.g., DNA-dependent DNA polymerases, RNA-dependent DNA polymerases, exonucleases, pyrophosphatases, etc.). Thus, in these mixtures, the portion of the polypeptide of the invention in the mixture may provide less than 50% of the enzymatic activity in the mixture, for example 45%, 35%, 33%, 30%, 25%, 20%, 15%, 10%, 7%, 5%, 2%, 1%, 0.5%, 0.1% of the total DNA-dependent DNA polymerase activity, RNA-dependent DNA polymerase activity, and/or exonuclease activity in the mixture.

Kits for cDNA synthesis may comprise a first container containing the wild type or mutant DNA polymerase of the invention, a second container may contain one up to four dNTPs and a third container may contain an oligo(dT) primer. See U.S. Pat. Nos. 5,405,776 and 5,244,797. Since the polypeptides of the invention, for example, the polypeptides of SEQ ID NOS:14-25, are also capable of preparing dsDNA, a fourth container may contain an appropriate primer complementary to the first strand cDNA. Kits of the invention may optionally comprise a container containing one or more DNA polymerase enzymes, for example, thermostable DNA polymerase enzymes such as Taq polymerase and/or reverse transcriptases (e.g., retroviral reverse transcriptases) and the like.

Of course, it is also possible to combine one or more of these reagents in a single tube or other containers. A detailed description of such formulations at working concentrations is described in the International patent application entitled “Stable Compositions for Nucleic Acid Amplification and Sequencing” filed on Aug. 14, 1996, WO 98/06736 which is expressly incorporated by reference herein in its entirety.

When desired, the kit of the present invention may include one or more containers that contain detectably labeled nucleotides that may be used during the synthesis or sequencing of a DNA molecule. One or a number of labels may be used to detect such nucleotides. Illustrative labels include, but are not limited to, radioactive isotopes, fluorescent labels, chemiluminescent labels, nuclear tags bioluminescent labels and enzyme labels.

### 10. Advantages of the Polypeptides of the Invention.

As discussed above, the polypeptides of the present invention provide a vast improvement in assays combining reverse transcription and amplification. The need to adjust buffer reaction conditions during the progression of the assay from reverse transcription to amplification is eliminated whether the same or a different enzyme is used for either part of the assay.

Having now generally described the invention, the same will be more readily understood through reference to the following Examples which are provided by way of illustration, and are not intended to be limiting of the present invention, unless specified.

### Example 1

### Cloning of Polypeptides of the Invention

DNA polymerase from Clostridium stercorarium cloned into the expression vector pET26B (Novagen Inc., Madison, Wis.) in the BL21SI cell line Invitrogen Corporation, Carlsbad, Calif.), obtained from Macquarie University was purified.

Conserved motifs found in known bacterial PolI DNA polymerase sequences were identified and degenerate PCR primers were designed for PCR amplification of an internal portion of polI genes from all bacterial divisions. We describe here a method that has allowed the rapid identification and isolation of 13 polI genes from a diverse selection of thermophilic bacteria and report on the biochemical characteristics of nine of the recombinant enzymes. Several enzymes showed significant Reverse Transcriptase activity in the presence of Mg²⁺.

Thermostable DNA polymerase from Thermus aquaticus (Taq) made the polymerase chain reaction (PCR) feasible, and introduced a powerful technology that complemented recombinant DNA studies and aided in the diagnosis of inherited and infectious diseases (Innis et al., 1990, In PCR Protocols: A Guide to Methods and Applications. Academic Press, San Diego.). Taq DNA polymerase also has reverse transcriptase activity (Jones and Foulkes, Nucleic Acids Res. 17, 8387-8388, 1989). The reverse transcriptase activity of a recombinant DNA polymerase from Thermus thermophilus (rTth, (Myers and Gelfand, Biochem. 30, 7661-7666, 1991) has been reported to be one hundred-fold greater than that of Taq DNA polymerase. The Taq and rTth enzymes have significant amino acid sequence similarity, and it is not clear why their abilities to utilize RNA templates are so different. Reverse transcription by thermophilic DNA polymerases has advantages over mesophilic retroviral reverse transcriptases (RTs) such as Moloney murine leukemia virus (M-MLV) and avian myeloblastosis virus (AMV) RT which are commonly used for cDNA synthesis, because the higher reaction temperatures with thermophilic polymerases help destabilize RNA secondary structures which pose problems for mesophilic RTs (DeStefano et al, J. Biol. Chem. 266, 7423-7431, 1991; Harrison et al, Nucleic Acids Res. 26, 3433-3442, 1998; Wu et al, J. Virol. 70, 7132-7142, 1996). The uses and advantages of using thermophilic DNA polymerases for reverse transcription and reverse transcription-coupled PCR amplifications (RT-PCR) have been described (Myers and Gelfand, 1991). However, one of the disadvantages of using rTth DNA polymerase for copying RNA is the requirement for the use of Mn²⁺, rather than Mg², as the divalent metal ion. The presence of Mn²⁺ results in a higher error rates during cDNA synthesis (Cadwell and Joyce, PCR Methods and Applications 2, 28-33, 1992) and in reduced yields of DNA product during PCR amplification (Leung et al, Technique 1, 11-15, 1989). Special measures must be taken during the PCR step of RT-PCR to remove the influence of Mn²⁺ introduced during the reverse transcription step (Myer and Gelfand, 1991).

Accordingly, we have carried out a survey of a number of thermophilic bacteria to identify DNA polymerases that could be used to copy RNA efficiently at elevated temperatures, exclusively in the presence of Mg²⁺. We have used degenerate oligonucleotide-based PCR (Rose et al., Nucleic Acids Res. 26, 1637-1644, 1998) combined with Genomic Walking PCR (Morris et al, Appl. Environ. Microbiol. 61, 2262-2269, 1998), to obtain the full-length gene sequences of 13 thermophilic polI genes. The degenerate primers were designed to hybridize to DNA coding for two conserved regions identified in an alignment of 24 bacterial PolI sequences. Three forward and three reverse primers were designed to amplify a PCR product of approximately 570 bp. The cloning of the genes, and the purification and preliminary characterization of the gene products are described here. We have identified several thermophilic DNA polymerases that copy RNA efficiently in the presence of Mg²⁺.

### Materials and Methods

Microorganisms. Clostridium stercorarium (Cst); Clostridium thermosulfurogenes (Cth); Caldibacillus cellulovorans CompA.2 (CA2); Caldicellulosiruptor sp. strain Tok13B.1 (Tok13B); Caldicellulosiruptor saccharolyticus sp. Tok7B. 1 (Tok7B); Caldicellulosiruptor sp. strain Rt69B.1 (RT69B); Bacillus caldolyticus EA1.3 (B.EA1); Thermus sp. Rt41A (RT41A) and Dictyoglomus thermophilum strain Rt46B.1 (Dth) were kindly supplied by Professor Hugh Morgan, Thermophile Research Unit, Waikato University, Hamilton, New Zealand.

Samples of E. coli BL21(DE3) transformed with a plasmid encoding the indicated polymerase has been deposited with the Agricultural Research Service Culture Collection (NRRL), 1815 North University Street, Peoria, Ill., 61604, USA in accordance with the Budapest Treaty. Entries 11-15 were deposited in E. coli BL21 (SI).

| | | | Strain Desig |
|---|---|---|---|
| | Origin of Polymerase | Abbr. | NRRL |
| 1 | Dictyoglomus thermophilum | Dicty | NRRL B-30617 |
| 2 | Bacillus caldolyticus EA1 | BEA1 | NRRL B-30618 |
| 3 | Thermoanaerobacter AZ3B.1 | AZ3B.1 | NRRL B-30619 |
| 4 | Caldicellulosiruptor Tok13B.1 | Tok13B.1 | NRRL B-30620 |
| 5 | Caldicellulosiruptor | Csac | NRRL B-30621 |
| | saccharolyticus | | |
| 6 | Thermus isolate Rt41A.1 | Rt41A.1 | NRRL B-30622 |
| 7 | Caldicellulosiruptor Tok7B.1 | Tok7B.1 | NRRL B-30623 |
| 8 | Caldicellulosiruptor Rt69B.1 | Rt69B.1 | NRRL B-30624 |
| 9 | Tepidomonas | Tepido | NRRL B-30625 |
| 10 | Spirochaete | Spiro | NRRL B-30626 |
| 11 | Caldibacillus cellulovorans | CompA.2 | NRRL B-30576 |
| | CompA.2 | | |
| 12 | Clostridium thermosulfurogenes | Cth | NRRL B-30577 |
| 13 | Clostridium thermosulfurogenes | Cth NtHis | NRRL B-30579 |
| 14 | Clostridium stercorarium | Cst | NRRL B-30578 |
| 15 | Clostridium stercorarium | Cst-His | NRRL B-30580 |
| | (N-terminal 6-His tag) | | |

Enzymes. Thermus aquaticus (Taq) DNA polymerase was from Invitrogen Corporation, Carlsbad, Calif. Recombinant Thermus thermophilus (rTth) DNA polymerase was purchased from Applied Biosystems (Foster City, Calif.).

Thermotoga neapolitana (Tne) DNA polymerase mutated to eliminate 3′ to 5′ and 5′ to 3′ exonuclease activity was cloned, engineered and purified as described in U.S. Pat. No. 6,306,588. SuperScript II reverse transcriptase (SS II RT) was from Invitrogen Corporation, Carlsbad, Calif.

RNA and DNA. Chloramphenicol acetyl transferase (CAT) cRNA (˜900 nt) with a (rA)₄₀ 3′-tail was synthesized by T7 RNA polymerase run-off transcription from linearized plasmid DNA (D'Alessio and Gerard, Nucleic Acids Res. 16, 1999-2014, 1988). Deoxyoligonucleotides were from Invitrogen Corporation, Carlsbad, Calif. cDNA synthesis from CAT cRNA was primed with a DNA 24mer complementary to CAT cRNA that annealed between nucleotides 679 and 692 with its 5′ end 146 nt distant from the first base at the 5′ end of the CAT cRNA (rA)₄₀ tail. (rA)₂₅₀ and (dA)₂₇₀ were from Amersham-Pharmacia (Piscataway, N.J.).

SDS-PAGE. Purified DNA polymerases were analyzed by SDS-PAGE. Approximately 1 μg of purified protein was loaded onto a 4-20% Tris-glycine gel (Novex, Invitrogen Corporation, Carlsbad, Calif.). The gel was run according to the manufacturer's recommendation and was stained using Gel-code Blue (Pierce, Rockford, Ill.). The Benchmark Protein Ladder was used as a standard (Invitrogen Corporation, Carlsbad, Calif.).

Removal of DNA from commercial polymerase preparations. Commercial preparations of recombinant Taq polymerase were found to contain trace amounts of DNA encoding the Taq polymerase gene (Carroll, et al., J. Clin. Microbiol. 37, 1999). To digest and remove the contaminating DNA, 2.5 units of the restriction enzyme Sau3AI were added to each 50 μl PCR reaction and the incubated at 37° C. for 30 minutes. The mixture was then heated to 95° C. for 2 minutes to denature the Sau3AI before adding approximately 1 ng of genomic template DNA.

PCR. PCRs were performed using Platinum Taq (Invitrogen Corporation, Carlsbad, Calif.) or Platinum Pfx (Invitrogen Corporation, Carlsbad, Calif.) according to the manufacturers recommendations. All PCRs were performed using a GeneAmp 2400 (Applied Biosystems), using 30 to 35 cycles and 50 to 70° C. annealing, unless stated otherwise. Genomic walking PCR to obtain full-length gene sequences was carried out as previously described (Morris, et al., 1995; Morris, et al., Appl Environ Microbiol 64(5):1759-65, 1998; Reeves, et al., Appl Environ Microbiol 66(4):1532-7, 2000). When required, PCR products were purified using a Concert gel extraction kit (Invitrogen Corporation, Carlsbad, Calif.).

When using degenerate primers in the PCR, a step-down method was used where the annealing temperature was lowered from 60° C. to 45° C. by 1° C. per cycle, followed by 35 cycles with a 55° C. annealing temperature.

Genomic walking to obtain full-length polymerase genes. Genomic walking linker libraries were prepared by digesting 2 μg of genomic DNA to completion in 20 μl, using 20 units of each of the following restriction enzymes: AatII, BamHI, EcoRI, EcoRV, HaeIII, HindIII, HpaI, KpnI, NcoI, PstI, PvuII, RsaI, SacI, SalI, SmaI, SphI, SspI, StuI or XbaI (from MBI Fermentas, Amherst, N.Y., or Roche Diagnostics, Sydney, Australia). The NcoI digested DNA was heat treated to 65° C. for 20 minutes to inactivate the restriction enzyme, as the recognition site for this enzyme is regenerated upon ligation to the linker. Half of each digest was ligated to the appropriate genomic walking linker (GW-linker, 1 μM concentration) using T4 ligase (MBI Fermentas) overnight at 10° C. in 20 μl. Portions of each digest/ligation were diluted to 10⁻¹ in TE buffer for use as PCR template. Gene-specific primers were designed to anneal approximately 50 bp in from the end of known sequence. Two series of the PCR were carried out in 50 μl volumes using either the forward or reverse gene specific primer, the appropriate linker specific primer and 1 μl of one of the diluted linker library template. The PCR program used included a 65-70° C. annealing temperature and a 2 minute extension step, allowing products of up to 2 kb to be amplified: 95° C., 15 minutes, 35 (95° C. 30 seconds, 70° C. 30 seconds, 72° C. 2 minutes) 72° C. 5 minutes. During this study, 13 DNA polymerases genes were isolated using this method, with sizes ranging from 2.5 kb to 2.8 kb, of which nine have been further characterized and are described herein.

Once the complete DNA sequence of each polI gene had been obtained, oligonucleotide primers were designed for specific amplification of each full-length gene. Restriction sites were incorporation into each primer to allow directional in-frame ligation of PCR product into the expression vector pET26B (Novagen Inc., Madison, Wis.). Each gene was PCR amplified using high fidelity Pfx DNA polymerase and purified from agarose gel following electrophoresis. The DNA was extracted from the gel and digested with the appropriate restriction enzymes to remove the ends of the primers, producing overhangs for ligation. The linear pET26B vector was treated with 2 U of Shrimp Alkaline Phosphatase (SAP, Roche) for 10 minutes at 37° C. to remove the 5′ phosphate and then heated to 65° C. for 15 min to inactivate the SAP. The DNA Polymerase gene (30 ng) was ligated into the linear vector and used to transform E. coli DH5α cells with selection on LB agar plates containing 30 μg/ml Kanamycin.

DNA sequencing, Computer analysis and GenBank Accession numbers. Plasmids and PCR products were sequenced using Perkin Elmer Big Dye Terminator chemistry and run on a Perkin Elmer ABI Prism 377 DNA sequencer.

Computer analysis of sequence data was carried out using the GCG software package (Devereux, 1984).

Subcloning of genes for Cst and Cth DNA polymerases. In order to improve expression and simplify purification of Cst and Cth DNA polymerase, the genes were subcloned downstream of a T7 promoter and an amino-terminal His₆ tag sequence was introduced using Gateway cloning technology (Invitrogen Corporation, Carlsbad, Calif.). The sequence of the DNA oligonucleotide used at the 5′ end of the Cst gene was: 5′-GGGGACAACTTTGTACAAAAAAGTTGTCGATCCAAAAATAATCCTT ATAGAC 3′ (SEQ ID NO:37). The sequence of the DNA oligonucleotide used at the 5′ end of the Cth gene was: 5′-GGGGACAACTTTGTACAAAAAAGTTGTCGCGAAATTT TTGATCATAGATGGT-3′ (SEQ ID NO:38). The sequence of the DNA oligonucleotide used at the 3′ end of each gene was the same: 5′-GGGGACAACTTTGTACAAGAAAGTTGCTCAGGAGGCTT CATACCAGTTTTT 3′ (SEQ ID NO:39). Purified pET26B plasmid DNA (Novagen Inc., Madison, Wis.) bearing the gene for Cst or Cth DNA polymerase was amplified by PCR utilizing the primers listed above and Platinum Taq HiFi DNA polymerase (Invitrogen Corporation, Carlsbad, Calif.). PCR products purified by agarose gel electrophoresis were cloned into Gateway vector pDON21 and transferred by recombination into vector pDEST17. This resulted in the introduction of a His₆ tag at the amino terminus of the Cst and Cth DNA polymerases and the positioning of a T7 promoter upstream of the genes. Each final recombinant plasmid was transformed into the E. coli expression host BL21-AI (Invitrogen Corporation, Carlsbad, Calif.).

Subcloning of genes for Tok13B, Tok7B, and Rt69B. Subcloning of the genes for Tok13B, Tok7B, and Rt69B DNA polymerase was carried out to remove the pelB leader sequence derived from pET26B. This reduced proteolytic degradation of the DNA polymerases from these genes observed in E. coli when the pelB leader was present. Each DNA polymerase gene was removed from pET26B by restriction digestion of the plasmid DNA with NcoI, which cut at the 5′ end of the gene, and BamHI, which cut downstream of the translation stop codon at the 3′ end of the gene. The NcoI-BamHI fragment was ligated into the NcoI and BamHI sites of expression vector pET14B (Novagen Inc., Madison, Wis.). The recombinant plasmids were transformed into the E. coli expression host BL21-AI (Invitrogen Corporation, Carlsbad, Calif.).

Purification of CA2, B.EA1, Rt41A, Dth, Tok13B, Tok7B, and Rt69B DNA polymerase. E. coli cells (BL21SI, Invitrogen Corporation, Carlsbad, Calif.) bearing the plasmid pET26B with the gene for CA2, B.EA1, Rt41A, or Dth DNA polymerase were grown in 2.8-1 Fembach flasks in LB broth containing no salt and 50 μg/ml kanamycin at 37° C. After the culture reached an A₅₉₀ of 1.2, expression of DNA polymerase was induced with 0.3 M NaCl for 3 hr. Cells were harvested by centrifugation and stored at −70° C. E. coli cells (BL21AI) bearing the plasmid pET14B with the gene for Tok13B, Tok7B, or RT69B DNA polymerase were grown in 2.8-1 Fembach flasks in LB broth containing 50 μg/ml ampicillin at 37° C. After the culture reached an A₅₉₀ of 1.0, expression of DNA polymerase was induced by the addition of 0.2% arabinose for 3 hr. Cells were harvested by centrifugation and stored at −70° C.

All purification steps were carried out at 4° C. or on ice unless stated otherwise. Frozen cells (7 gm) were thawed and suspended in sonication buffer (50 mM Tris-HCl, pH 7.5, 1 mM EDTA, 8% (v/v) glycerol, 5 mM β-mercaptoethanol, and 50 μg/ml PMSF) at a 1:3 ratio (w/v) of buffer. The cell suspension was sonicated until greater than 70% of the total cells were lysed. A 10% (v/v) solution of NP-40 and Tween 20 was added to the sonicated sample to a final concentration of 0.05% of each. The sonicated sample was heated at 55° C. (CA2 and B.EA1 DNA polymerase), 60° C. (Tok13B, Tok7B, and RT69B) or 75° C. (Dth and RT41A DNA polymerase) for 15 min then cooled on ice for 30 min. NaCl (5 M) was added to a final concentration of 0.25 M and polymin P was added to a final concentration of 0.2%. The sample was centrifuged at 20,000×g for 20 min to remove the precipitate. Solid ammonium sulfate was dissolved in the supernatant (0.326 gm/ml) and the suspension was stirred overnight. The insoluble protein was collected by centrifugation and resuspended in 5 ml of low salt buffer [25 mM Tris-HCl (pH 8.0), 50 mM NaCl, 0.5 mM EDTA, 5% (v/v) glycerol, 2 mM β-mercaptoethanol and 0.05% (v/v) each of NP-40 and Tween 20. The sample was dialyzed against 200 ml of the low salt buffer and centrifuged to remove insoluble material. The protein was fractionated by column chromatography on a 5-ml EMD sulfate (EM Sciences, address ?) column in low salt buffer eluted with a linear gradient of 50 mM to 500 mM NaCl. The fractions containing DNA polymerase were determined by SDS-PAGE analysis and assay for DNA-directed DNA polymerase activity. These were pooled and dialyzed overnight against the low salt buffer. The dialyzed protein was fractionated by column chromatography on a MonoQ HR 5/5 column (Amersham Pharmacia) run in low salt buffer and eluted using a linear gradient of 50 mM to 250 mM NaCl. Fractions containing the thermostable DNA polymerase were pooled and dialyzed overnight against storage buffer [20 mM Tris-HCl (pH 8.0), 40 mM KCl, 0.1 mM EDTA, 50% (v/v) glycerol, 1 mM DTT, 0.04% (v/v) each of NP-40 and Tween 20]. Purified DNA polymerase was stored at −20° C.

Purification of Cst-His and Cth-His DNA polymerases. E. coli cells (BL21AI) bearing the plasmid pDEST17 with the gene for Cst-His or Cth-His DNA polymerase were grown in 2.8-1 Fembach flasks in LB broth containing 50 μg/ml ampicillin at 37° C. After the culture reached an A₅₉₀ of 1.0, expression of DNA polymerase was induced by the addition of 0.2% arabinose for 3 hr. Cells were harvested by centrifugation and stored at −70° C.

All operations were at 4° C. unless otherwise specified. Frozen cells (7 gm) were thawed and suspended at a 1:2 ratio (w/v) in 50 mM Tris-HCl (pH 7.8), 10% (v/v) glycerol, and 2 mM MgCl.sub.2. Cells were disrupted by sonication and Benzonase® (E. Merck, address ?) was added at a ratio of 25 U per mL of slurry. After 30 mM, NaCl was added to a final concentration of 1 M. The suspension was centrifuged at 13,000×g for 30 mM. The crude extract was fractionated by column chromatography on a 5-mL HiTrap™ chelating column charged with Ni²⁺ and washed in 25 mM Tris-HCl (pH 7.8), 1 M NaCl, 5 mM imidazole, and 10% (v/v) glycerol (buffer N). After loading the sample, the column was washed in buffer N containing 20 mM imidazole and eluted with a linear gradient from 20 mM to 450 mM imidazole. Fractions were assayed for DNA-directed DNA polymerase activity and the peak fractions were pooled. EDTA was added to the pooled fractions to a final concentration of 1 mM and the pool was dialyzed against 25 mM Tris-HCl (pH 8.0), 50 mM NaCl, 0.5 mM EDTA, 5% (v/v) glycerol, and 1 mM β-mercaptoethanol (buffer H). The dialyzed pool was fractionated on a 1- or 5-mL HiTrap Heparin column (Amersham Pharmacia) equilibrated in buffer H. After loading the sample, the column was washed with buffer H and eluted with a linear gradient of 50 mM to 800 mM NaCl. The fractions were assay for DNA polymerase activity and the peak fractions were pooled. The pooled fractions were dialyzed against 20 mM Tris-HCl (pH 8.0), 40 mM KCl, 0.1 mM EDTA, 50% (v/v) glycerol, and 1 mM DTT. The final sample was stored at −20° C.

DNA polymerase activity assays. DNA-directed DNA polymerase unit activity-Reaction mixtures (50 μl) contained 25 mM TAPS (pH 9.3), 2.0 mM MgCl₂, 50 mM KCl, 1.0 mM DTT, 0.2 mM each of dATP, dTTP, dGTP, and [α-³²P]dCTP (250 cpm/pmole), 500 μg/ml activated salmon testes DNA, and 2 to 4 pg (0.02 to 0.2 pmoles) DNA polymerase. After incubation at 55 or 72° C. for 10 mM, the reaction was terminated by addition of 10 μl of 0.5 M EDTA. Incorporation of radioactivity into acid-insoluble DNA product was determined. One unit of DNA-directed DNA polymerase activity is the amount of enzyme required to incorporate 10 nmoles of dNTPs into acid insoluble product in 30 mM.

RNA-directed DNA polymerase unit activity. Reaction mixtures (25 μl) contained 10 mM Tris-HCl (pH 8.3), 25 mM KCl, 5 mM MgCl₂, 0.5 mM each of dATP, dTTP, dGTP, and [α-³²P]dCTP (200 cpm/pmole), 1. mu.g (3.2 pmoles) CAT cRNA, and 0.6 μg (80 pmoles) DNA 24mer primer. The range of the amount of DNA polymerase used in the assay varied. For CA2, Cst-His and B.EA1 DNA polymerases, 0.25 to 4 DNA-directed DNA polymerase units were used and the reaction was incubated at 55° C. for 5 min. For Cth-His DNA polymerase, 5 to 40 DNA-directed DNA polymerase units were incubated at 55° C. for 5 min. In the case of Tok13B, Tok7B, RT69B, Dth, and RT41A DNA polymerases, the range was 5 to 40 DNA-directed DNA polymerase units incubated at 72° C. for 5 min. The reaction was terminated by addition of 5 μl of 0.5 M EDTA. Incorporation of radioactivity into acid-insoluble DNA products was determined. One unit of RNA-directed DNA polymerase activity is the amount of enzyme required to incorporate 10 nmoles of dNTPs into acid insoluble product in 30 min.

Reverse transcriptase functional activity. Reaction mixtures (20 μl) contained 10 mM Tris-HCl (pH 8.3), 25 mM KCl, 5 mM MgCl₂, 0.5 mM each of dATP, dTTP, dGTP, and [α-³²P]dCTP (200 cpm/pmole), 1 μg CAT cRNA, and 0.6 μg DNA 24mer primer. The reaction was set up in the presence and absence of 1.5 M betaine. The amount of DNA polymerase activity (DNA-directed DNA polymerase units) added to the reaction was: 1 unit of CA2, 5 units of Cst-His, 20 units of Cth-His, or 10 units of B.EA1, Tok13B, Tok7B, RT69B, Dth, RT41A, Tne, rTth, or Taq DNA polymerase. SUPERSCRIPT™ II RT (200 units) was incubated as a control at 42° C. and the other enzymes were incubated at 60° C. for 30 min. A portion of the reaction mixture was precipitated with TCA to determine total yield of cDNA synthesized, and the remaining cDNA product was size fractionated on an alkaline 2% agarose gel. The gel was dried and exposed to X-ray film.

Thermal inactivation profiles of DNA polymerases. Purified DNA polymerases were analyzed for thermostability at temperatures between 55 and 95° C. A reaction mixture (50 μl) containing 10 mM Tris-HCl (pH 8.3), 25 mM KCl, 5 mM MgCl₂, and 2.5 units of DNA-directed DNA polymerase activity was incubated at various temperatures for 10 min. The tubes were placed on ice and 5 μl of the sample was tested for residual DNA polymerase activity using the DNA-directed DNA polymerase unit activity assay. After incubation at 55° C. (DNA polymerases CA2, Cst-His, B.EA1 and Cth-His) or 72° C. (DNA polymerases Tok13B, Tok7B, RT69B, Dth and RT41A) for 10 min, the reaction was terminated by addition of 5 μl of 0.5 M EDTA. Incorporation of radioactivity into acid-insoluble DNA products was determined.

Steady-state kinetic measurements. The steady-state kinetic parameters Kₘ(dTTP) and k_{CAT} were determined as described (Polesky et al., J. Biol. Chem. 265, 14579-14591,1990) using (rA)₂₅₀.(dT)₃₀ or (rA)₂₅₀.(dT)₄₀ and (dA)₂₇₀.(dT)₄₀. A range of four to five [³²P]dTTP concentrations, which bracketed the Kₘ(dTTP) value, was used for Kₘ(dTTP) determinations. Reaction mixtures (50 μl) contained 10 mM Tris-HCl (pH 8.3), 25 mM KCl, 5 mM MgCl₂, 100 to 1,000 μM [α-³²P]dTTP, 1 μM (rA)₂₅₀ or (dA)₂₇₀, 3 μM (dT)₃₀ or (dT)₄₀, and 5 to 50 nM DNA polymerase. In some cases, k_{CAT} was determined with (dC)ₙ.(dG)₃₅ (Astalke et al., J. Biol. Chem. 270, 1945-1954, 1995) in reaction mixtures (50 μl) containing 20 mM Tris-HCl (pH 8.4), 50 mM KCl, 2 mM MgCl₂, 100 to 200 μM [α-³²P]dGTP, and 5 nM DNA polymerase.

### Results and Discussion

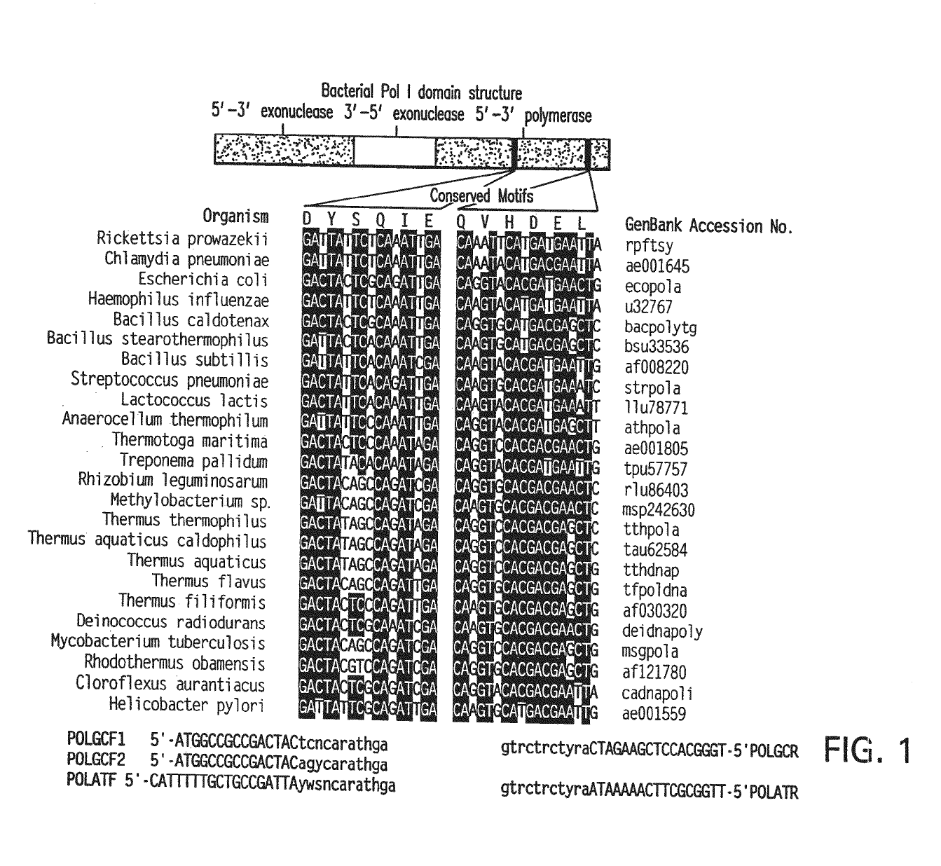

Cloning of DNA polymerase genes: Degenerate Oligonucleotide Design. The amino acid sequences from 24 bacterial Pol I DNA polymerases were aligned and two highly conserved regions were identified within the 5′-3′ DNA polymerase domain of all enzymes (FIG. 1.) Consensus-degenerate hybrid oligonucleotide primers (CODEHOP, Rose et al., 1998) were designed to hybridize to DNA coding for the conserved regions. The DNA sequences of the polymerases identified are SEQ ID NOS: 2, 3, 4, 5, 6, 7, 8, 9, 10, 11, 12 and 13, and the corresponding amino acid sequences are SEQ ID NOS:14, 15, 16, 17, 18, 19, 20, 21, 22, 23, 24 and 25. Three forward and three reverse primers were designed to amplify a PCR product of approximately 570 bp (see FIG. 1.) The PolGCF1/F2 and PolGCR primers were found to work best with organisms with a high % G+C content. The PolGCF1 and PolGCF2 primers are identical apart from the sequence encoding the serine codon positioned within the motif. The primers PoIATF and R were based upon the sequences of the PolGCF1/F2 and PolGCR primers but with a lower % G+C within the 5′-nondegenerate end of each primer. Decreasing the % G+C content of the non-degenerate ends was found to improve the correct amplification of polI genes from organisms with a low % G+C content.

The degenerate primers were then designed for use in a step-down PCR protocol with a decrease in annealing temperature by 1° C. per cycle, starting from 60° C. down to 45° C. This was followed by 35 cycles of amplification with an annealing temperature of 55° C. The degenerate primers described in FIG. 1 were used to amplify internal portions of polI genes from the following bacteria: Caldicellulosiruptor saccharolyticus, Caldicellulosiruptor saccharolyticus strains, Tok7B.1, Rt69B.3 and Tok13B.1; Thermus thermophilus strain Rt41.A; Dictyoglomus thermophilum strain Rt46B. 1; Clostridium stercorarium; Clostridium (Thermoanaerobacterium) thermosulfurigenes; Thermoanaerobacter sp. AZ3B.1; Bacillus caldolyticus strain EA1; and Caldibacillus cellulovorans CompA.2. The degenerate primer combination that amplified the internal portion of each polymerase gene is shown in Table 33. In terms of correct amplification of the internal polymerase gene region, there was a direct correlation between the % G+C content of template genomic DNA and the % G+C content of the non-degenerate 5′ portion of the CODEHOP primers. The PolATF/R primer combinations were required for correct amplification of polI from low % G+C genomic DNA, while the PolGCF1/F2/R primers worked most efficiently with high % G+C genomic DNA.

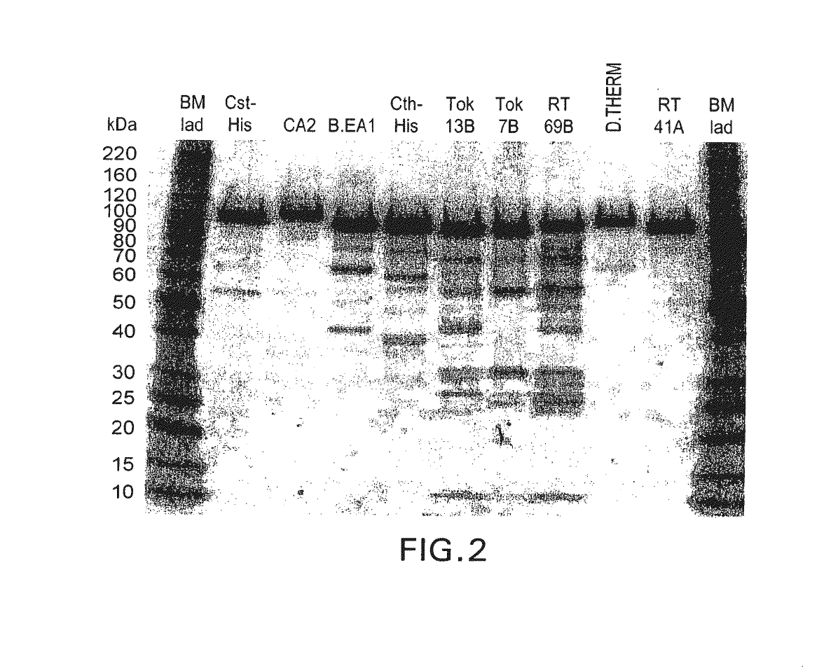

Purification. Proteins were expressed and purified as described and analyzed by SDS-PAGE. The results are shown in FIG. 2. The Cst-His, CA2, Dth, and RT41A polymerases were approximately 90% homogenous, the approximately B.EA1 and Cth-His polymerases were approximately 80% homogeneous, and the Tok13B, Tok7B, and RT69B were approximately 70% homogeneous.

Thermal Stability. There appear to be three classes of polymerase based on thermal stability. As seen in Table 38, a first class comprising Cth-His, CompA.2, Cst-His, and B. EA1 are highly active at 60° C. and may maintain their activity to 65° C. but appear to be inactive at temperatures of about 70° C. and higher. A second class comprising Tok13B, Tok7B, and RT69B, appear to be maximally active at temperatures of about 70° C. to about 75° C. and to maintain their activity to about 80 degrees but to have lower activity at temperatures higher than about 80° C. A third class of polymerase comprising Dth and RT41A appear to be maximally active at temperatures from about 75° C. to about 90° C. and to maintain detectable activity at temperatures as high as 95° C.

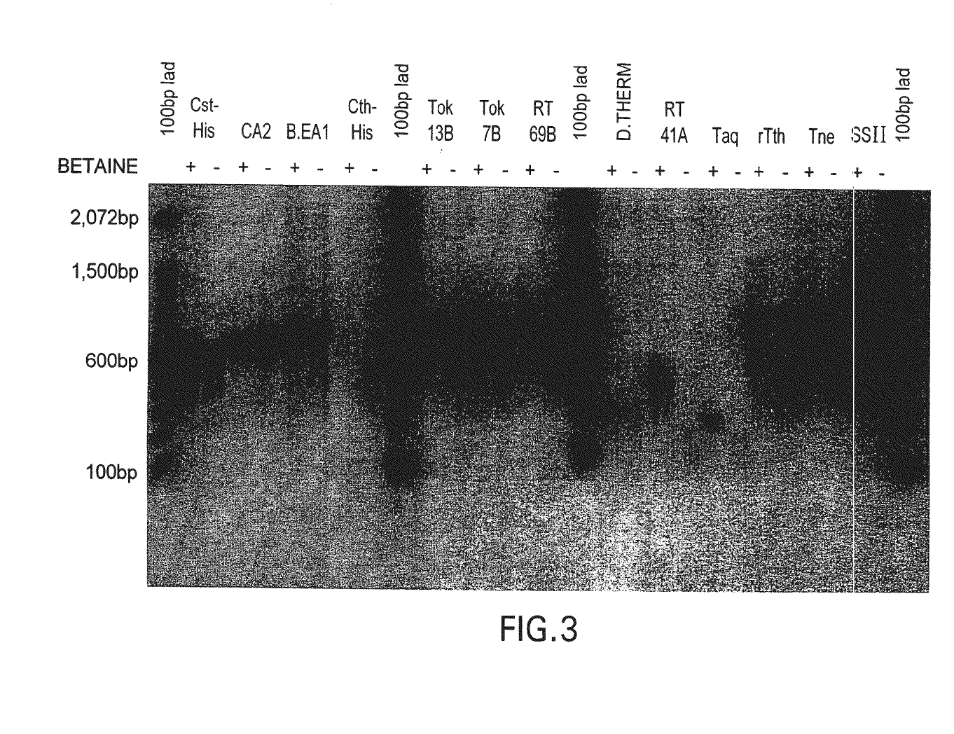

Reverse Transcriptase Activity. With reference to FIG. 3 and Tables 39 and 40, the present invention identifies three classes of polymerase with regard to RNA-dependent DNA polymerase activity. The first class exemplified by Taq, RT41A and Dth have little or no detectable reverse transcriptase activity. The members of the second class, exemplified by recombinant Tth, Tok7B, Cth-His, RT69B, Tok13B, and Tne, have a demonstrable reverse transcriptase activity but at a low level. Polymerases of this class may have a specific activity level for RNA-dependent DNA polymerase activity of from about 20 to about 350 units/mg of protein. A third class of polymerase enzymes identified by the present invention may have a specific activity for RNA-dependent DNA polymerase activity of greater than about 500 units/mg. In some embodiments, the present invention provides polymerases having a specific activity for RT activity of greater than 1,000 units/mg, greater than about 1,500 units/mg, greater than 2,000 units/mg, greater than about 2,500 units/mg, greater than about 3,000 units/mg, greater than about 3,500 units/mg, greater than bout 4,000 units/mg, greater than about 4,500 units/mg, greater than about 5,000 units/mg, greater than about 7,500 units/mg or greater than about 10,000 units/mg.

The RT specific activity of the polymerases of the invention may be influenced by the reaction conditions, for example, the inclusion of additives such as betaine may influence the observed RT activity. With reference to FIG. 3, the first strand reaction of various polymerases was compare with and without the addition of betaine to the reaction mixture. Some enzymes, (e.g., rTth and Tne) appear to require the presence of betaine in order to produce a full length product.

Careful design of degenerate primers for the initial PCR of the consensus polI sequence allowed the amplification and sequencing of an internal gene fragment which allowed the design of gene specific primers suitable for genomic walking in the 5′ and 3′ directions so that the entire polI gene could be isolated from a variety of bacteria with widely differing % G+C contents, but it was necessary to design a suite of primers to achieve successful amplifications. The high conservation of the motifs against which the degenerate primers were designed means that theoretically, these primers should amplify the polI genes from the majority of bacteria across all bacterial divisions. The degenerate PCR method was so sensitive that initial difficulties were encountered due to the presence of trace amounts of the Taq polymerase gene in commercial enzyme preparations. We found it was necessary to pre-treat Taq enzyme with a temperature-sensitive restriction enzyme to remove the contaminating Taq polI DNA. This method has the advantage over isolation of polI genes from genomic expression libraries as no demands are made for expression in the host E. coli, which may cause weakly expressed PolA enzymes to be overlooked. Accordingly, the genes can be translated in appropriate expression vectors under optimal conditions for the production of the particular enzyme.

### Example 2

### Growth and Expression

The constructs were analyzed for expression of the DNA polymerase. Overnight cultures were grown (2 ml) in LB no salt (LBON) containing kanamycin (50 μg/ml) at 37° C. To 40 ml of LBON+Kan, 1 ml of the overnight culture was added and the culture was grown at 37° C. until it reached an O.D of ˜1.0 (A₅₉₀). The culture was split into two 20 ml aliquots and the first aliquot (uninduced) was kept at 37° C. To the other aliquot, 5 M NaCl was added to a final concentration of 0.3 M and the culture was incubated at 37° C. After 3 hours the cultures were centrifuged at 4° C. in a tabletop centrifuge at 3500 rpm for 20 minutes. The supernatant was poured off and the cell pellet was stored at −70° C. until analyzed.

The expressed protein was analyzed by SDS-PAGE. The cell pellet was suspended in 1 ml of sonication buffer (10 mM Tris pH 8.0, 1 mM Na₂EDTA, 10 mM β-mercaptoethanol (β-ME)) and was sonicated (550 Sonic Dismembrator (Heat Systems), ½ inch tip, at a setting of 8 with 10 sec pulse for a total of 100 seconds). The sonicated sample was clarified by centrifugation. The supernatant (crude lysate) was used for the analysis of the soluble proteins. Samples (amount equivalent to 0.1 A₅₉₀ units) were loaded on a 4-20% gradient Tris-glycine gel. Samples were run under reduced condition using Tris-glycine SDS buffer.

### Example 3

### Measuring DNA Polymerase Activity

The crude lysate was analyzed for thermostable polymerase activity. An aliquot of the crude lysate was placed either in a 55° C. or a 75° C. water bath and heated for 15 minutes. Each sample was cooled on ice, centrifuged to bring down precipitated proteins, and each supernatant was analyzed for thermostable DNA-dependent DNA polymerase activity. The activity assay is a 25 μl reaction mixture containing 25 mM TAPS, pH 9.3, 2.0 mM MgCl₂, 50 mM KCl, 1.0 mM DTT, 0.2 mM each dNTP, 12.5 μg nicked salmon testes DNA, and 1 μCi ³H-TTP. After incubation at 72° C. for 10 minutes, the reaction was terminated by addition of 5 μl of 0.5 M EDTA. Incorporation of radioactivity into acid-insoluble products was determined.

Thermostable DNA-dependent DNA polymerase activity was seen in the crude lysate as well as in the 55° C. heat denatured samples of all three polymerases. However the 75° C. heat denatured samples of C. stercorarium and C. thermosulfurogenes polymerases lost greater than 95% of their activity, while the Caldibacillus cellulovorans CompA.2 polymerase lost greater than 90% of its activity.

### Example 4

### Expression and Purification of Thermostable DNA Polymerase

Cells were grown on a large scale in shake flasks. For each clone, two 20 ml cultures of LBON+Kan were inoculated using a glycerol seed. The culture was then grown overnight at 37° C. Fifteen ml of overnight culture was added to 750 ml of LBON+Kan mixture, and incubated at 37° C. Following cell growth (A₅₉₀˜1.2) the cultures were induced with NaCl (0.3M final concentration) and were grown for three more hours. The cells were harvested by centrifugation and stored at −70° C.

All steps were carried out at 4° C. or on ice unless stated otherwise. The cells containing the recombinant plasmid (about 7 grams) were thawed and suspended in the sonication buffer (1:3 ratio of cells to buffer in 50 mM Tris, pH 7.5, 1 mM Na₂EDTA, 8% glycerol, 5 mM β-ME), and 50 μg/ml PMSF). The cell suspension was sonicated (550 Sonic Dismembrator, ½ inch tip, at a setting of 8 with 10 sec pulse for a total of 100 seconds) until greater than 70% of the total cell fraction was lysed (determined by A.sub.590 measurement). A 10% solution of NP-40 and Tween® 20 (polyethylene(20)sorbitan monolaurate) was added to the sonicated sample to a final concentration of 0.05%. The sonicated sample was heated at 55° C. for 15 minutes and then cooled on ice for 30 minutes. A solution of 5M NaCl was added to a final concentration of 0.25M and the sample was stirred. This was followed by the dropwise addition of a 5% solution of polyethylenimine (PEI) to a final concentration of 0.2%, with constant stirring. The suspension was stirred for an additional 30 minutes. The sample was then centrifuged at 13,000 rpm at 4° C. in an SS 34 rotor for 20 minutes to remove precipitated nucleic acids. Solid ammonium sulfate was added to the supernatant (0.326 gm/ml) and the suspension was stirred overnight. The pellet was collected by centrifugation and re-suspended in 5 ml of low salt buffer containing 25 mM Tris, pH8.0, 50 mM NaCl, 0.5 mM Na₂EDTA, 5% glycerol, 2 mM β-ME and 0.05% each NP-40 and Tween® 20. This is also the buffer used in the wash and the gradient.

The sample was dialyzed against 200 ml of the low salt buffer. Following centrifugation to remove any insoluble materials, the protein was loaded on a 5 ml EMD sulfate (EM Sciences) column and was eluted by a linear gradient of 50 mM to 500 mM NaCl in low salt buffer. The fractions containing the thermostable DNA polymerase were determined by SDS-PAGE and DNA polymerase activity assay (see below). These selected fractions were pooled and dialyzed overnight against the low salt buffer. The dialyzed sample was loaded on a MonoQ HR 5/5 column (Amersham/Pharmacia) and the protein was eluted using a linear gradient of NaCl from 50 mM to 250 mM. The fractions containing the thermostable DNA polymerase were identified by SDS-PAGE and DNA polymerase activity assay. These were pooled and dialyzed overnight against dialysis buffer containing 20 mM Tris, pH 8.0, 40 mM KCl, 0.1 mM Na₂EDTA, 50% glycerol, 1 mM DTT, 0.04% NP-40 and 0.04% Tween.RTM. 20.

### Example 5

### Unit Assay for Measuring Thermostable DNA Polymerase Activity

The activity assay is a 50 μl reaction mixture containing 25 mM TAPS, pH 9.3, 2.0 mM MgCl₂, 50 mM KCl, 1.0 mM DTT, 0.2 mM each dNTP, 25 μg nicked salmon testes DNA, and 1 μCi [α-³²P]-dCTP. After incubation at 72° C. for 10 minutes, the reaction was terminated by addition of 10 μl of 0.5 M EDTA. Incorporation of radioactivity into acid-insoluble products was determined.

### Example 6

### Reverse Transcriptase (RT) Activity in the Presence of Manganese (Mn.Sup.+2)

Purified polypeptides of the invention were assayed for RT activity. SUPERSCRIPT™ II (Invitrogen, Carlsbad, Calif.) and rTth DNA polymerase (Perkin Elmer, Wellesley, Mass.) were also used as controls. Five units (DNA polymerase unit) of the polypeptide of the invention was added to a 20 μl reaction containing 10 mM Tris, pH 8.3, 90 mM KCl, 1 mM MnCl₂, 0.2 mM of each dNTP, 0.05% each of NP-40 and Tween® 20, 1 μg of total CAT-RNA, 0.6 μg of a gene specific primer (GSP1) and 2 μCi of [α-³²P] dCTP. The reaction for each polypeptide was incubated at one of the temperatures: 55° C., 60° C., 65° C. or 70° C. for 30 minutes. The reaction was terminated by addition of 5 μl of 0.5M NaEDTA. Incorporation of radioactivity into acid-insoluble products was determined. Clostridium stercorarium showed good incorporation of radioactivity at all the temperatures.

The same reaction was repeated at 60° C. with samples of Clostridium stercorarium polymerase, Clostridium thermosulfurogenes polymerase, Caldibacillus cellulovorans CompA.2 polymerase, SUPERSCRIPT™ II and rTth DNA polymerase and analyzed for cDNA synthesis by alkaline agarose gel electrophoresis. Clostridium stercorarium, Clostridium thermosulfurogenes, Caldibacillus cellulovorans CompA.2, SUPERSCRIPT™ II and rTth were all able to synthesize the 700 bp cDNA.

### Example 7

### Reverse Transcriptase (RT) Activity in the Presence of Magnesium (Mg⁺²)

Reactions were set up at three different concentrations of Mg⁺² and dNTP. They were 1 mM Mg⁺²/0.2 mM dNTP (five fold excess of Mg⁺²), 3 mM Mg⁺²/0.5 mM dNTP (six fold excess of Mg⁺²), and 7.5 mM Mg⁺²/1 mM dNTP (seven and one-half fold excess of Mg⁺²). The rest of the components were the same as for the RT activity assay in the presence of manganese. cDNA synthesis as measured by incorporation of radioactivity was seen with Clostridium stercorarium and SUPERSCRIPT™ II with the six fold excess Mg⁺² reaction being the best.

The reaction was repeated at 60° C. with samples of Clostridium stercorarium polymerase, Clostridium thermosulfurogenes polymerase, and SUPERSCRIPT™ II and analyzed for cDNA synthesis by alkaline agarose gel electrophoresis. In this trial, only Clostridium thermosulfurogenes and SUPERSCRIPT™ II were able to synthesize the fall length cDNA of 700 bp. However Clostridium stercorarium showed the synthesis of smaller cDNA products (≈100 to 300 bp).

Caldibacillus cellulovorans CompA.2 polymerase was assayed as described above using SUPERSCRIPT™ II and rTth as controls. The reaction components were the same as for the RT activity in the presence of manganese except for two components. The reaction had 3 mM Mg⁺² instead of 1 mM MnCl₂ and the dNTP concentration was 0.5 mM. Incorporation of radioactivity into acid-insoluble products was determined and the sample was analyzed for cDNA synthesis by alkaline agarose gel electrophoresis. Both Caldibacillus cellulovorans CompA.2 and SUPERSCRIPT™ II were able to synthesize the full length cDNA of≈700 bp. No radioactive incorporation or cDNA synthesis was observed with rTth.

### Example 8

### Reverse Transcriptase (RT) Activity in the Presence of Magnesium (Mg⁺²) and Betaine

The reaction mix was the same as above except for betaine was titrated into the reaction mixture (no betaine, 0.1 M, 0.5 M, 1.0 M and 1.5 M final concentration). cDNA synthesis was analyzed by alkaline agarose gel electrophoresis. With Clostridium stercorarium, the ˜700 bp cDNA product was synthesized in reactions containing 1.0M and 1.5M betaine. In the absence of betaine ˜200 bp fragment was seen and in the presence of 0.5M betaine ˜400 bp fragment was synthesized. With Clostridium thermosulfurogenes the full length≈700 bp cDNA was synthesized in reactions containing no betaine and 0.1M betaine. The higher concentrations of betaine seemed to inhibit full length cDNA synthesis with most of the products being less than≈500 bp. In the presence of 5% DMSO, Clostridium stercorarium was observed to synthesize ˜400 bp −500 bp fragments.

### Example 9

### Construction of Sub-Clones

The clones were generated by using the Gateway™ cloning technology (Invitrogen, Carlsbad, Calif.). Clones with either a native amino terminal sequence or a histidine tagged amino terminal sequence were created. The oligonucleotide used to generate the amino terminal of each clone is different whereas the carboxy terminus oligonucleotide is the same. The sequences of the oligonucleotides used to generate the Clostridium stercorarium clones were as follows:

Native amino terminal
(SEQ ID NO: 40)
5′-GGGGACAACTTTGTACAAAAAAGTTGTCAGGAGGTTAACCATGGAT
CCAAAAATAATCCTTATAGAC-3′
Histidine tagged amino terminal
(SEQ ID NO: 41)
5′-GGGGACAACTTTGTACAAAAAAGTTGTCGATCCAAAAATAATCCTT
ATAGAC-3′
Carboxy terminal
(SEQ ID NO: 39)
5′-GGGGACAACTTTGTACAAGAAAGTTGCTCAGGAGGCTTCATACCAG
TTTTT-3′

The sequences of the oligonucleotides used to generate the Clostridium thermosulfurogenes clones were as follows:

Native amino terminal
(SEQ ID NO: 41)
5′-GGGGACAACTTTGTACAAAAAAGTTGTCAGGAGGTTAACCATGG
CGAAATTTTTGATCATAGATGG-3′
Histidine tagged amino terminal
(SEQ ID NO: 38)
5′-GGGGACAACTTTGTACAAAAAAGTTGTCGCGAAATTTTTGATCA
TAGATGGT-3′
Carboxy terminal
(SEQ ID NO: 42)
5′-GGGGACAACTTTGTACAAGAAAGTTGCTTATTTTGCATCA
TACCAGTTTTT-3′

Plasmid DNA (the polymerase cloned in the pET26B vector) was isolated from the original clones. This was used as the template for a PCR reaction using either the native or His tagged N-terminal primer with the carboxy terminal primer. Each 100 μl reaction contained 1× HiFi PCR reaction buffer, 0.2 mM dNTPs, 2 mM MgSO₄, 5 units of PLATINUM® Taq HiFi, 0.2 μM each primer and 5 μl of template DNA. PCR cycling was 2-min initial denaturation at 94° C. followed by 25 cycles of 30 sec. at 94° C., 30 sec. at 57° C., and 2.4 minutes at 68° C.

The PCR products were analyzed on an agarose gel and the products were purified. The product was cloned into the pDONR201 vector by following the BP reaction protocol listed in the Gateway™ manual from Invitrogen Corporation, Carlsbad, Calif. Twenty microliters of the BP reaction was used to conduct an LR reaction by following the one tube protocol in the Gateway manual. In the LR reaction the vector pDEST 14 was used for generating the native clone and the vector pDEST17 was used in generating the amino terminus His-tag clones. One microliter of the LR reaction was transformed into Max-efficiency DH10B cells and the cells were plated on LB plates containing ampicillin. After incubation at 37° C. the colonies were analyzed for the presence of the recombinant clone by restriction enzyme digest. The recombinant plasmid was then transformed into the expression host BL21-BAD.

Cells were grown at 30° C. overnight. These were used for inoculating larger cultures. The large scale cultures were grown at 37° C. until they reached on O.D of≈1.0 (A₅₉₀) and then were induced by adding arabinose to a final concentration of 0.2%. The cells were allowed to grow for an additional three hours. Cells were harvested by centrifugation and stored at −70° C.

Polymerase was purified from the native amino terminal clones as described above. Polymerase was purified from the histidine tagged clones using nickel affinity chromatography.

### Example 10

### Determination of Optimum Mg⁺² Concentration for RT Activity

Samples of Caldibacillus cellulovorans CompA.2 polymerase and histidine-tagged Clostridium stercorarium and Clostridium thermosulfurogenes polymerases were analyzed to determine the optimal Mg⁺² concentration for RT activity for each enzyme.

Two units (DNA polymerase unit at 55° C.) of each enzyme was analyzed in a 20 μl reaction containing 10 mM Tris, pH 8.3, 90 mM KCl, 0.5 mM each dNTP, 2 μg of total CAT-RNA, 0.6 μg of a gene specific primer (GSP1), and 2 μCi of α-³²P dCTP. In addition, the reactions of the Caldibacillus cellulovorans CompA.2 and the Clostridium stercorarium polymerases contained 1.5 M betaine. The final concentration of Mg⁺² was titrated from 1 mM to 30 mM (specifically 1 mM, 3 mM, 5 mM, 7.5 mM, 10 mM, 15 mM, 20 mM, 25 mM, 30 mM). Samples were incubated at 60° C. for 15 min. The reactions were terminated by addition of 5 μl of 0.5 M EDTA.

Incorporation of radioactivity into acid-insoluble products was determined. Five millimolar Mg⁺² was seen to be the optimal amount.

### Example 11

### Determination of Optimum KCl Concentration for RT Activity

Samples of Caldibacillus cellulovorans CompA.2 polymerase and histidine-tagged Clostridium stercorarium and Clostridium thermosulfurogenes polymerases were analyzed to determine the optimal KCl concentration for RT activity for each enzyme.

Two units (DNA polymerase unit at 55° C.) of each enzyme was analyzed in a 20 μl reaction containing 10 mM Tris, pH 8.3, 5 mM MgCl₂, 0.5 mM of each dNTP, 2 μg of total CAT-RNA, 0.6 μg of a gene specific primer (GSP1), and 2 μCi of [α-³²P]dCTP. In addition, the reactions of the Caldibacillus cellulovorans CompA.2 and the Clostridium stercorarium polymerases contained 1.5 M betaine. The final concentration of KCl was titrated from 0 mM to 125 mM (specifically 0 mM, 25 mM, 50 mM, 75 mM, 100 mM, and 125 mM). Samples were incubated at 60° C. for 15 min. The reactions were terminated by addition of 5 μl of 0.5 M EDTA. Incorporation of radioactivity into acid-insoluble products was determined. A KCl concentration of 25 mM was seen to be the optimal amount. Activity was considerably lower at the higher KCl concentrations.

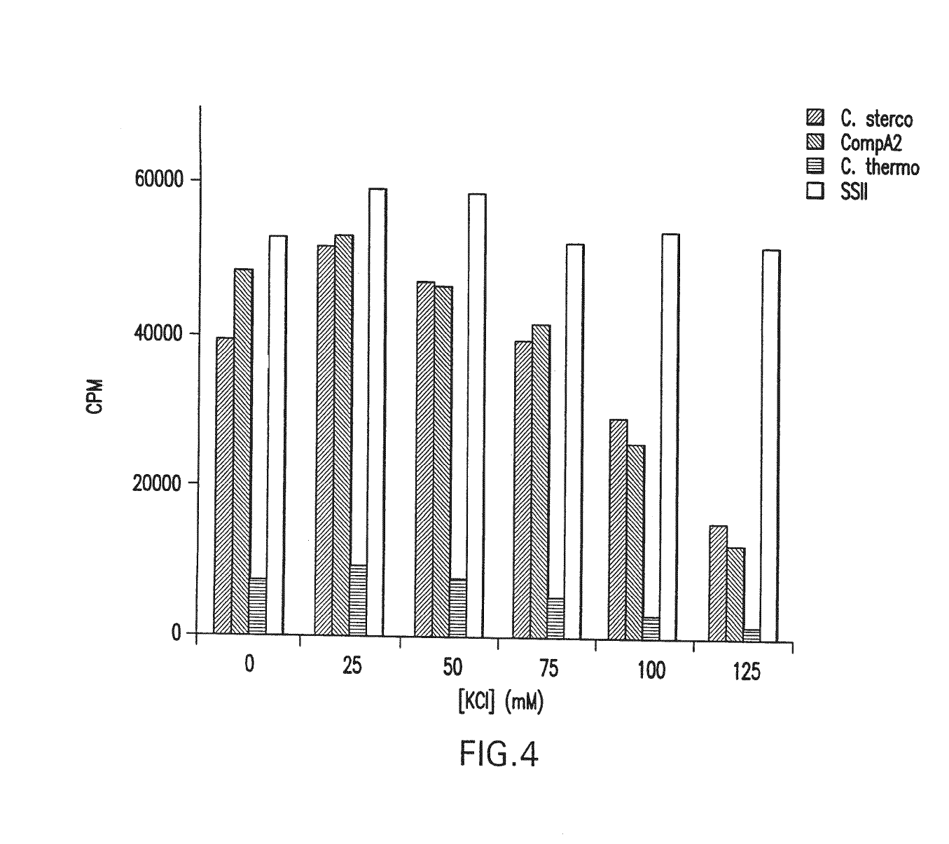

With reference to FIG. 4, in buffer with a lower salt concentration (e.g., 25 mM KCl), Mg-dependent RT activities of the polymerases increased at least 2 fold from those in high salt buffer (e.g., 90 mM KCl), while a viral reverse transcriptase enzyme (e.g., SUPERSCRIPT™ II) did not show salt dependency. RT activity was measured by incorporation of nucleotides using a CAT mRNA template primed with a gene specific primer (GSP) at 60° C. for 15 min. (or 30 min. for Clostridium thermosulfurogenes to compensate for slow reaction).

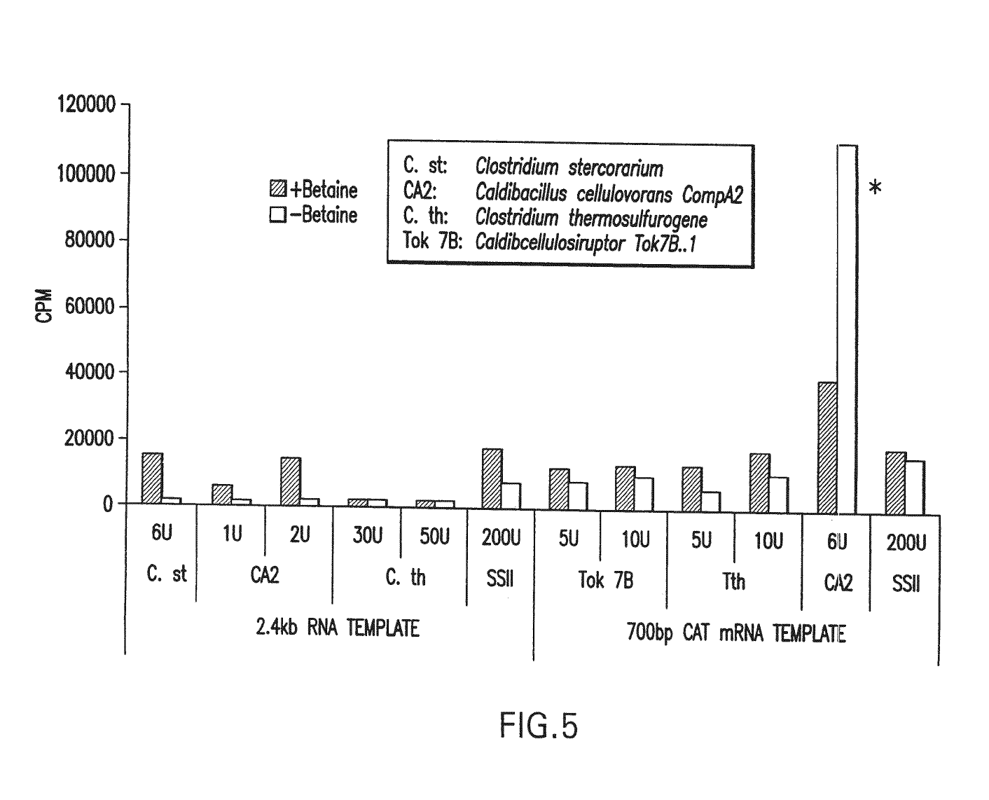

FIG. 5 shows the results of a comparison of the reverse transcriptase activity of varying amounts of the polymerases of the invention in the presence and absence of Betaine in low salt buffer. RT activity was measured by incorporation of nucleotides using a CAT mRNA template primed with GSP or 2.4 kb RNA template with oligo(dT) as primer, at 60° C. for 15 min (or 30 min. for Clostridium thermosulfurogenes to compensate for slow reaction).

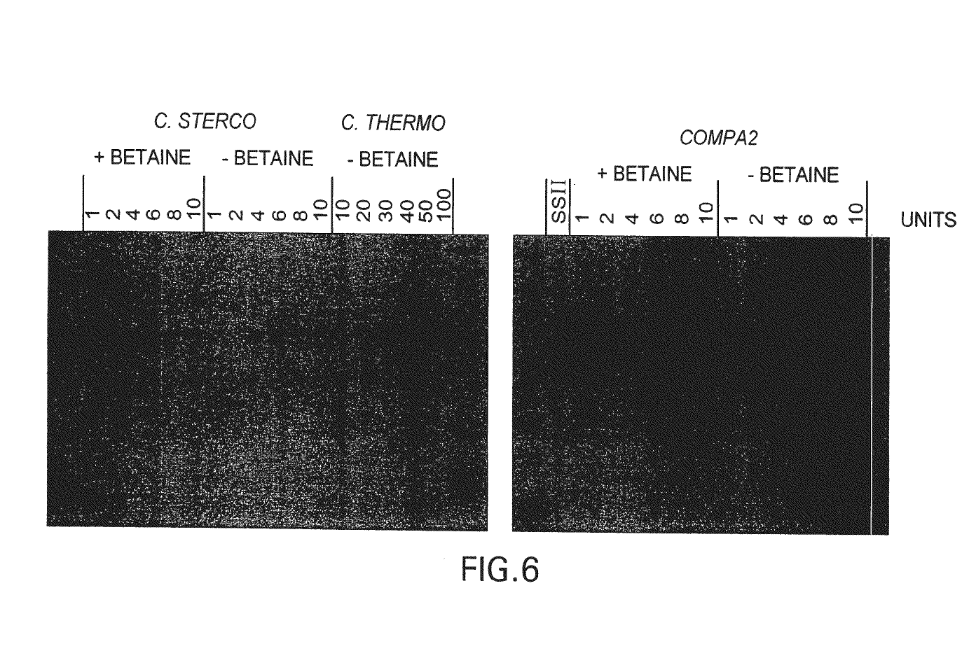

FIG. 6 is an autoradiograph of reverse transcriptase activity of several polymerases of the invention in the presence and absence of Betaine in low salt buffer. Reverse transcriptase activity of the DNA polymerase from Clostridium stercorarium becomes Betaine-dependent in low salt buffer (e.g., 25 mM KCl) at enzyme concentrations higher than 4 U/rxn. Reverse transcriptase activity was measured by incorporation of nucleotides using a CAT mRNA template primed with a GSP at 60° C. for 15 min (or 30 min. for Clostridium thermosulfurogenes to compensate for slow reaction). The polymerase from Caldibacillus cellulovorans CompA.2 has higher specificity in presence of 1.5 M Betaine.

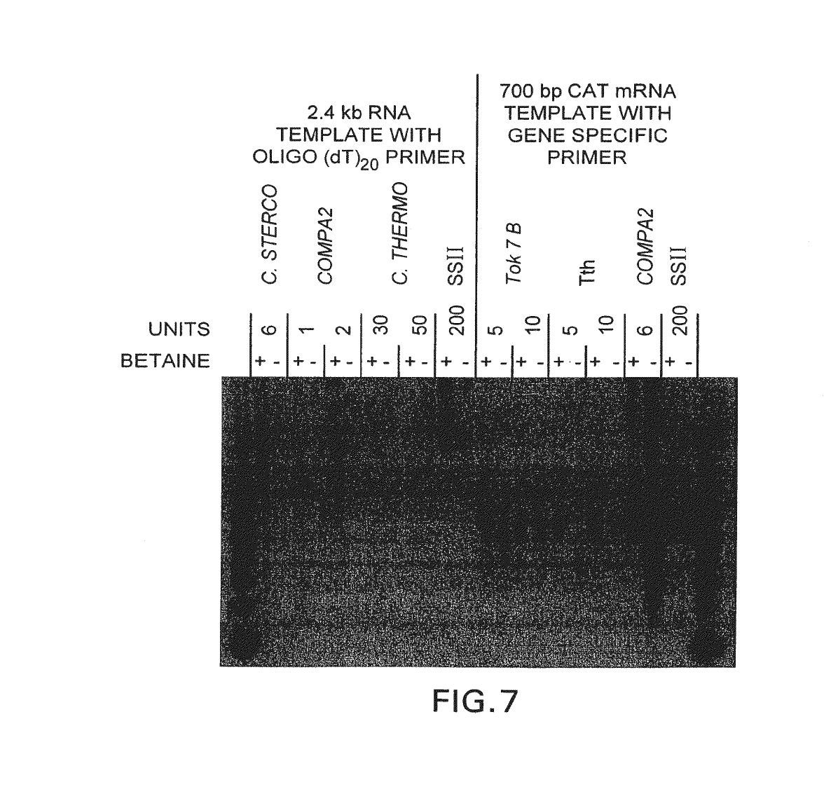

FIG. 7 is an autoradiograph showing reverse transcriptase activity of several polymerases of the invention in the presence and absence of Betaine. Reverse transcripatase activity was measured by incorporation of nucleotides using a CAT mRNA template primed with GSP or 2.4 kb RNA template with oligo(dT) as primer, at 60° C. for 15 min (or 30 min. for Clostridium thermosulfurogenes to compensate for slow reaction). This result shows that a lower RT activity of some polymerases may attribute to initiation step where they show a lower affinity to DNA oligo-primed RNA templates.

### Example 12

### Determination of Optimum pH for RT Activity

Samples of Caldibacillus cellulovorans CompA.2 polymerase and histidine-tagged Clostridium stercorarium and Clostridium thermosulfurogenes polymerases were analyzed to determine the optimal pH for RT activity for each enzyme.

Two units (DNA polymerase unit at 55° C.) of each enzyme was analyzed in a 20 μl reaction containing 10 mM Tris, pH 8.3, 5 mM MgCl₂, 25 mM KCl, 0.5 mM of each dNTP, 2 μg of total CAT-RNA, 0.6 μg of a gene specific primer (GSP1), and 2 μCi of [α-³²P]dCTP. In addition, the reactions of the Caldibacillus cellulovorans CompA.2 and the Clostridium stercorarium polymerases contained 1.5 M betaine. Tris buffers at pH 7.2, pH 7.5, pH 8.0, pH 8.3, and pH 8.8 were used at a final concentration of 10 mM.

Samples were incubated at 60° C. for 15 min The reactions were terminated by addition of 5 μl of 0.5 M EDTA. Incorporation of radioactivity into acid-insoluble products was determined. A slight increase in activity was seen from pH 7.2 through pH 8.8. pH 8.3 was taken to be optimal. Polymerases of the invention may be used at a pH of from about 7.0 to about 9.0, from about 7.2 to about 9.0, from about 7.5 to about 9.0, from about 7.8 to about 9.0, from about 8.0 to about 9.0, from about 8.2 to about 9.0, from about 8.3 to about 9.0, from about 8.4 to about 9.0, from about 8.5 to about 9.0, from about 8.6 to about 9.0, from about 8.7 to about 9.0, from about 8.8 to about 9.0, from about 8.9 to about 9.0, from about 8.0 to about 8.9, from about 8.0 to about 8.8, from about 8.0 to about 8.7, from about 8.0 to about 8.6, from about 8.0 to about 8.5, from about 8.0 to about 8.4, from about 8.0 to about 8.3, from about 8.0 to about 8.2, from about 8.0 to about 8.1, from about 8.2 to about 8.6, from about 8.2 to about 8.5, from about 8.2 to about 8.4, or from about 8.2 to about 8.3.

### Example 13

### Determination of Optimum Amount of Enzyme for RT Activity

Samples of Caldibacillus cellulovorans CompA.2 polymerase and histidine-tagged Clostridium stercorarium and Clostridium thermosulfurogenes polymerases were analyzed to determine the optimal amount of enzyme for RT activity for each enzyme.

The reactions for Caldibacillus cellulovorans CompA.2 polymerase and histidine-tagged Clostridium stercorarium were set up in the presence and absence of 1.5 M betaine, the Clostridium thermosulfurogenes reaction did not include betaine. The 20 μl reactions contained 10 mM Tris-HCl pH 8.3, 25 mM KCl, 5 mM MgCl₂, 0.5 mM of each dNTP, 1 μg of total CAT-RNA, 0.6 μg of a gene specific primer (GSP1), and 2 μCi of [α-³²P]-dCTP. The range of enzyme used was 1 unit, 2 units, 4 units, 6 units, 8 units, and 10 units (DNA polymerase unit at 55° C.) for the Caldibacillus cellulovorans CompA.2 polymerase and the histidine-tagged Clostridium stercorarium polymerase and 10 units, 20 units, 30 units, 40 units, 50 units, and 100 units for the Clostridium thermosulfurogenes polymerase. Samples were incubated at 60° C. for 60 min. The reactions were terminated by addition of 5 μl of 0.5 M EDTA. Incorporation of radioactivity into acid-insoluble products was determined.

Alkaline agarose gel analysis of the cDNA products showed that both in the presence and absence of betaine, even 1 unit of the enzyme was sufficient to give full length product (700 bp) with either the Caldibacillus cellulovorans CompA.2 polymerase or the histidine-tagged Clostridium stercorarium polymerase. In the absence of betaine, 4 units of the histidine-tagged Clostridium stercorarium polymerase was sufficient to produce full length product. The inclusion of 20 units of the Clostridium thermosulfurogenes polymerase was sufficient to produce full length products.

### Example 14

### cDNA Synthesis of 2.4 kb RNA

One and two units of Caldibacillus cellulovorans CompA.2 polymerase, six units Clostridium stercorarium polymerase and thirty and sixty units of Clostridium thermosulfurogenes polymerase were used to reverse transcribe a 2.4 kb RNA. The reactions for Caldibacillus cellulovorans CompA.2 polymerase and histidine-tagged Clostridium stercorarium were set up in the presence and absence of 1.5 M betaine, the Clostridium thermosulfurogenes reaction did not include betaine. The 20 μl reactions contained 10 mM Tris-HCl pH 8.3, 25 mM KCl, 5 mM MgCl₂, 0.5 mM dNTP, 1 μg of 2.4 kb RNA, 50 pmoles of oligo (dT) 20 and 2 μCi of [α-³²P]-dCTP. Samples were incubated at 50° C. for 5 min followed by incubation at 60° C. for 60 minutes. The reactions were terminated by addition of 5 μl of 0.5 M EDTA. Incorporation of radioactivity into acid-insoluble products was determined. Alkaline agarose gel analysis of the cDNA products showed that with 2 units of the Caldibacillus cellulovorans CompA.2 enzyme and six units Clostridium stercorarium polymerase, in the presence of betaine, full-length product was obtained. The Clostridium thermosulfurogenes polymerase did not produce full length product under these conditions.

### Example 15

### Use of Enzyme in RT-PCR

Clostridium thermosulfurogenes DNA polymerase, Clostridium stercorarium DNA polymerase and Caldibacillus cellulovorans CompA.2 DNA polymerase (5 units of each enzyme) were used in conjunction with PLATINUM® Taq DNA polymerase in one step RT-PCR. In addition to the components indicated above, each 50 μl reaction volume contained: 1×PCR buffer (10 mM Tris-HCl pH 8.3, 90 mM KCl), 1.2 mM MgCl₂, 0.2 mM each dNTP, 100 ng of total CAT RNA, 10 pmole CAT forward primer (CGA CCG TTC AGC TGG ATA TTA C (SEQ ID NO:43)), 10 pmole of CAT reverse primer (TTG TAA TTC ATT AAG CAT TCT GCC (SEQ ID NO:44)), and 2.5 units of PLATINUM.RTM. Taq DNA polymerase. The reactions were incubated at 60° C. for 30 min followed by 2 min at 95° C., 40 cycles of 95° C. for 15 sec., 55° C. for 30 sec., 72° C. for 45 sec., followed by 72° C. for 2 minutes. The product was resolved on a 1% agarose gel stained with ethidium bromide. The expected 520 bp fragment was observed with all three enzymes.

The Clostridium stercorarium DNA polymerase was used in conjunction with PLATINUM® Taq DNA polymerase in one step RT-PCR. The following components were assembled in a 50 μl reaction volume: 1×PCR buffer (10 mM Tris-HCl pH 8.3, 90 mM KCl), 1.2 mM MgCl₂, 0.2 mM each dNTP, 100 ng of total CAT RNA, 10 pmole CAT forward primer (CGA CCG TTC AGC TGG ATA TTA C (SEQ ID NO:43)), 10 pmole of CAT reverse primer (TTG TAA TTC ATT AAG CAT TCT GCC (SEQ ID NO:44)), 1.5 mM betaine, 2.5 units of PLATINUM® Taq DNA polymerase and 5 units of Clostridium stercorarium DNA polymerase. The reaction was incubated at 60° C. for 30 min followed by 2 min at 95° C., 40 cycles of 95° C. for 15 sec., 55° C. for 30 sec., 72° C. for 45 sec., followed by 72° C. for 2 minutes. The product was resolved on a 1% agarose gel stained with ethidium bromide. The expected 520 bp fragment was observed.

The Caldibacillus cellulovorans CompA.2 DNA polymerase was used in conjunction with PLATINUM® Taq DNA polymerase in one step RT-PCR. The following components were assembled in a 50 μl reaction volume: 1×PCR buffer (10 mM Tris-HCl pH 8.3, 90 mM KCl), 1.2 mM MgCl₂, 0.2 mM each dNTP, 100 ng of total CAT RNA, 10 pmole CAT forward primer (CGA CCG TTC AGC TGG ATA TTA C (SEQ ID NO:43)), 10 pmole of CAT reverse primer (TTG TAA TTC ATT AAG CAT TCT GCC (SEQ ID NO:44)), 2.5 units of PLATINUM® Taq DNA polymerase and 5 units of Caldibacillus cellulovorans CompA.2 DNA polymerase. The reaction was incubated at 60° C. for 30 min followed by 2 min at 95° C., 40 cycles of 95° C. for 15 sec., 55° C. for 30 sec., 72° C. for 45 sec., followed by 72° C. for 2 minutes. The product was resolved on a 1% agarose gel stained with ethidium bromide. The expected 520 bp fragment was observed.

### Example 16

### Kinetic Analysis of DNA-Dependent and RNA-Dependent Polymerase Activity

The catalytic rate constant k_{cat} and the Michaelis constant K_{M} were determined for both the DNA-dependent and RNA-dependent polymerase activities for the polypeptides of the invention and these parameters were compared to those of Tne DNA polymerase enzyme and SUPERSCRIPT™ II reverse transcriptase. The results of this analysis are summarized in Table 34. The assays were conducted in the presence of 1.5 mM MgCl₂ at 55° C. for all enzymes except the Caldibacillus cellulovorans CompA.2 enzyme where 2 mM MgCl₂ and 45° C. were used.

### Example 17

### Analysis of Reverse Transcriptase Activity and Thermal Stability for Selected Eubacterial Thermostable DNA Polymerases

The reverse transcriptase activity and thermal stability of a number of eubacterial DNA polymerase enzymes was determined and the results are summarized in Table 35. RT activity was determined with either Mn²⁺ or Mg²⁺.

The column headed Mn²⁺ shows the efficiency of synthesis of ³²P labeled full-length cDNA from CAT mRNA at 60° C. in the absence of additives under sub-optimal conditions.

The column headed Mg²⁺ shows the efficiency of synthesis of ³²P labeled full-length cDNA from CAT mRNA at 60° C. in the absence of additives under optimal conditions. The numbers in parentheses are the units required under optimal conditions to produce full-length CAT cDNA (700 bp) in the presence of Mg⁺⁺.

### Example 20

### Construction of N-Terminal and/or C-Terminal Deletion Mutants

The following general approach may be used to clone a N-terminal or C-terminal deletion mutant. Generally, two oligonucleotide primers of about 15-25 nucleotides are derived from the desired 5′ and 3′ positions of a polynucleotide of any one of SEQ ID NOS: 2-13. The 5′ and 3′ positions of the primers are determined based on the desired polynucleotide fragment. An initiation and stop codon are added to the 5′ and 3′ primers respectively, if necessary, to express the polypeptide fragment encoded by the polynucleotide fragment. Preferred polynucleotide fragments are those encoding the N-terminal and C-terminal deletion mutants and those encoding the 10, 11, 12, 13, 14, 15, 16, 17, 18, 19, 20, 21, 22, 23, 24, 25, 26, 27, 28, 29, 30, 31, 32, 33, and 34 amino acid fragments disclosed above.

Additional nucleotides containing restriction sites to facilitate cloning of the polynucleotide fragment in a desired vector may also be added to the 5′ and 3′ primer sequences. The polynucleotide fragment is amplified from genomic DNA or from the deposited clone using the appropriate PCR oligonucleotide primers and conditions discussed herein or known in the art. The polypeptide fragments encoded by the polynucleotide fragments of the present invention may be expressed and purified in the same general manner as the full length polypeptides, although routine modifications may be necessary due to the differences in chemical and physical properties between a particular fragment and full length polypeptide.

### Example 21

### Protein Fusions

Polypeptides of the invention may be fused to other proteins. These fusion proteins can be used for a variety of applications. For example, fusion to His-tag, HA-tag, protein A, IgG domains, and maltose binding protein facilitates purification. (See Example 5; see also EP A 394,827; Traunecker, et al., Nature 331:84-86 (1988).) Similarly, fusion to IgG-1, IgG-3, and albumin increases stability. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule.

Briefly, the Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the Fc portion is re-restricted with BamHI, linearizing the vector, and polynucleotide of the invention, amplified by PCR and isolated, is ligated into this BamHI site. Note that the polynucleotide is cloned without a stop codon, otherwise a fusion protein will not be produced.

The vector can also be modified to include a heterologous signal sequence. (See, e.g., WO 96/34891.)

Additionally, one or more components, motifs, sections, parts, domains, fragments, etc., of the polypeptides of the invention may be recombined with one or more components, motifs, sections, parts, domains, fragments, etc. of one or more heterologous molecules. In preferred embodiments, the heterologous molecules are clamps.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

TABLE 33

| | PolA | | | | |
|---|---|---|---|---|---|
| | Length | % | CODEHOP Primers Used | Restrictions | |
| Source | (amino | G + C | to Amplify Internal | Sites Used | PolA DNA |
| Organism | acids) | Content | Sequence | for Cloning | SEQ ID NO |
| Dictyoglomus | 856 | 33% | PolATF-PolATR | BamHI-SalI | SEQ ID NO: 10 |
| thermophilum | | | (SEQ ID NOs: 118, 120) | | |
| Caldicellulosiruptor | 849 | 33.3% | PolATF-PolATR | NcoI-BamHI | SEQ ID NO: 11 |
| saccharalyticus | | | (SEQ ID NOs: 118, 120) | | |
| Caldicellulosiruptor | 849 | 34.2% | PolATF-PolATR | NcoI-BamHI | SEQ ID NO: 6 |
| Tok7B.1 | | | (SEQ ID NOs: 118, 120) | | |
| Caldicellulosiruptor | 849 | 34.4% | PolATF-PolATR | NcoI-BamHI | SEQ ID NO: 7 |
| Rt69B.3 | | | (SEQ ID NOs: 118, 120) | | |
| Caldicellulosiruptor | 849 | 34.5% | PolATF-PolATR | NcoI-BamHI | SEQ ID NO: 5 |
| Tokl 3B.1 | | | (SEQ ID NOs: 118, 120) | | |
| Clostridium | 867 | 35% | PolATF-PolATR | NcoI-BamHI | SEQ ID NO: 3 |
| thermosulfurogenes | | | (SEQ ID NOs: 118, 120) | | |
| Clostridium | 898 | 44% | PolATF-PolATR | NcoI-BamHI | SEQ ID NO: 2 |
| stercorarium | | | (SEQ ID NOs: 118, 120) | | |
| Bacillus caldolyticus | 878 | 46.5% | PolGCF1-PolGCR | NcoI-BamHI | SEQ ID NO: 8 |
| EA1 | | | (SEQ ID NOs: 116, 119) | | |
| Caldibacillus | 904 | 64% | PolGCF1-PolGCR | NcoI-BamHI | SEQ ID NO: 4 |
| cellulovarans | | | (SEQ ID NOs: 116, 119) | | |
| CompA.2 | | | | | |
| Thermophilic | 898 | 65% | PolGCF1-PolGCR | EcoRI-SalI | SEQ ID NO: 12 |
| Spirochaete | | | (SEQ ID NOs: 116, 119) | | |
| Tepidomonas sp. | 928 | 68% | PolGCF1-PolGCR | EcoRI-SalI | SEQ ID NO: 13 |
| | | | (SEQ ID NOs: 116, 119) | | |
| Thermus Rt 41A | 833 | 68.3% | PolGCF1-PolGCR | EcoRI-SalI | SEQ ID NO: 9 |
| | | | (SEQ ID NOs: 116, 119) | | |

TABLE 34
Kinetic analysis of the DNA and RNA-dependent polymerase activities of
various polymerases

| | Kcat | Km | | Kcat | Km | |
|---|---|---|---|---|---|---|
| | s−1 | (μM) | | s−1 | (μM) | |
| | DNA | DNA | Kcat/Km | RNA | RNA | Kcat/Km |
| Protein | (dGTP) | (dGTP) | Efficiency | (dTTP) | (dTTP) | Efficiency |
| Tne | 17.5 | 17.2 | 1 | 0.094 | 37 | 0.0025 |
| Cst-His | 11.5 | 0.5 | 23 | 19.2 | 76 | 0.25 |
| Cth-His | 57 | 17.1 | 3.4 | 0.94 | 68 | 0.014 |
| CompA.2 | 200* | | | 5 | 17 | 0.3 |
| Peak2A | | | | | | |
| SSII | 15.4 | 2.4 | 6.4 | 14.5 | 17 | 0.85 |

*Assay done in the presence of 1.5 mM MgCl₂ at 55° C. The others were assayed in the presence of 2 mM MgCl₂ at 45° C.

TABLE 35
Correlation of Reverse Transcriptase Activity, Thermal Stability and Conserved Amino Acid Sequence
of a Select Group of Eubacterial Thermophilic DNA Polymerases

| | RT Activity | | Thermal Stability | | | | Amino Acid Motif |
|---|---|---|---|---|---|---|---|
| Origin | Mn^{++a} | Mg^{++b} | 60° C. | 70° C. | 80° C. | 90° C. | ry x₈ F x₃SFaer |
| Thermus aquaticus | -- | -- | +++ | +++ | +++ | +++ | ryvpdlearyKsvrEAaer |
| | | | | | | | (SEQ ID NO: 46) |
| Thermus RT 41A | -- | -- | +++ | +++ | +++ | ++ | ryvpdlasrvRsvrEAaer |
| | | | | | | | (SEQ ID NO: 47) |
| Thermatoga neopolitina | + | ++ | +++ | +++ | +++ | +++ | rdipqlmardKntqSEger |
| | | (10)^{c} | | | | | (SEQ ID NO: 48) |
| Thermus thermophilus | ++ | ++ | +++ | +++ | +++ | +++ | ryvpdlnaryKsvrEAaer |
| | | (10) | | | | | (SEQ ID NO: 49) |
| Dictyoglomus thermophilum | + | -- | +++ | +++ | +++ | ++ | ryipeiksinKqvrNAyer |
| | | | | | | | (SEQ ID NO: 50) |
| Caldocellulosiruptor | + | -- | ++ | -- | -- | -- | ryikdikstnKnlrNYaer |
| saccharolyticus | | | | | | | (SEQ ID NO: 51) |
| Caldocellulosiruptor | ++ | ++ | +++ | +++ | ++ | -- | ryikdikstnKnlrNYaer |
| Tok13B.1 | | (10) | | | | | (SEQ ID NO: 52) |
| Caldocellulosiruptor | ++ | ++ | +++ | +++ | ++ | -- | ryikdikstnKnlrNYaer |
| Tok7B | | (10) | | | | | (SEQ ID NO: 53) |
| Caldocellulosiruptor RT69B | ++ | ++ | +++ | +++ | ++ | -- | ryikdikstnKnlrNYaer |
| | | (10) | | | | | (SEQ ID NO: 54) |
| Bacillus caldolyticus EA1 | +++ | +++ | +++ | ++ | -- | -- | rylpditsrnFnvrSFaer |
| | | (10) | | | | | (SEQ ID NO: 55) |
| Clostridium | +++ | +++ | +++ | +/- | -- | -- | ryipeinsknFhqrSFgkr |
| thermosulfurogenes | | (20) | | | | | (SEQ ID NO: 56) |
| Clostridium stercorarium | +++ | +++ | +++ | + | -- | -- | rylpelasknFhqrSFgkr |
| | | (4) | | | | | (SEQ ID NO: 57) |
| Caldibacillus cellulovarans | +++ | +++ | +++ | + | -- | -- | rylpdinasnYnlrSFaer |
| CompA.2 | | (1) | | | | | (SEQ ID NO: 58) |

^{a}Efficiency of synthesis of ³²P-labeled full-length cDNA from CAT mRNA at 60° C. in the absence of additives under sub-optimal conditions.
^{b}Efficiency of synthesis of ³²P-labeled full-length cDNA from CAT mRNA at 60° C. in the absence of additives under optimal conditions.
^{c}The numbers in parentheses are the units required under optimal conditions to produce full-length CAT cDNA (700 bp) in the presence of Mg⁺⁺

TABLE 37
Representative sequences of Q-helices

| Pol AA | | Starting | |
|---|---|---|---|
| SEQ ID NO | Organism | AA # | RY X₈ F X₃SFaer |
| SEQ ID NO: 14 | Clostridium stercorarium | 815 | RYlpelasknFhqrSFgkr |
| | | | (SEQ ID NO: 57) |
| SEQ ID NO: 15 | Clostridium thermosulfurogene | 784 | RYipeinsknFhqrSFgkr |
| | | | (SEQ ID NO: 56) |
| SEQ ID NO: 16 | Caldibacillus cellulovorans | 820 | RYlpdinasnYnlrSFaer |
| | | | (SEQ ID NO: 58) |
| SEQ ID NO: 17 | Caldicellulosiruptor TOK13B.1 | 766 | RYikdikstnKnlrNYaer |
| | | | (SEQ ID NO: 52) |
| SEQ ID NO: 18 | Caldicellulosiruptor Tok7B.1 | 766 | RYikdikstnKnlrNYaer |
| | | | (SEQ ID NO: 53) |
| SEQ ID NO: 19 | Caldicellulosiruptor Rt69B.1 | 766 | RYikdikstnKnlrNYaer |
| | | | (SEQ ID NO: 54) |
| SEQ ID NO: 20 | Bacillus caldolyticus EA1 | 795 | RYlpditsrnFnvrSFaer |
| | | | SEQ ID NO: 55 |
| SEQ ID NO: 21 | Thermus Rt41A | 759 | RYvpdlasrvRsvrEAaer |
| | | | (SEQ ID NO: 47) |
| SEQ ID NO: 22 | Dictyoglomus thermophilum | 779 | RYipeiksinKqvrNAyer |
| | | | (SEQ ID NO: 50) |
| SEQ ID NO: 23 | Caldicellulusiruptor | 766 | RYikdikstnKnlrNYaer |
| | saccharalyticus | | (SEQ ID NO: 51) |
| SEQ ID NO: 24 | Spirochaete | 823 | RplpyitsrnKtqkTGaer |
| | | | (SEQ ID NO: 60) |
| SEQ ID NO: 25 | Tepidomonas | 854 | RLylpeiqspNgprRAaaer |
| | | | (SEQ ID NO: 61) |
| SEQ ID NO: 27 | Thermus aquaticus | 728 | RYvpdlearyKsvrEAaer |
| | | | (SEQ ID NO: 46) |
| SEQ ID NO: 29 | Thermus thermophilus | 730 | RyvpdlnaryKsvrEAaer |
| | | | (SEQ ID NO: 49) |
| SEQ ID NO: 30 | Thermoanaerobacter AZ3B.1 | 751 | RYipeinsrnFtqrSQaer |
| | | | (SEQ ID NO: 62) |
| SEQ ID NO: 32 | Bacillus stearothermophilus | 771 | RYlpditsrnFnvrSFaer |
| | | | (SEQ ID NO: 63) |
| SEQ ID NO: 33 | Bacillus caldotenax | 772 | RYlpditsrnFnvrSFaer |
| | | | (SEQ ID NO: 64) |
| SEQ ID NO: 34 | Escherichia coli | 823 | lylpdikssnGarrAAaer |
| | | | (SEQ ID NO: 65) |

TABLE 38
Thermal inactivation profiles of purified DNA polymerases^{a}

| | Percent activity^{b} remaining after heating for 10 min at the | | | | | | | | |
|---|---|---|---|---|---|---|---|---|---|
| | temperatures (° C.) below | | | | | | | | |
| Enzyme | 55 | 60 | 65 | 70 | 75 | 80 | 85 | 90 | 95 |
| Cth-His | 90 | 60 | 1 | 0 | —^{c} | — | — | — | — |
| CA2 | 109 | 101 | 47 | 0 | — | — | — | — | — |
| Cst-His | 95 | 94 | 81 | 0 | — | — | — | — | — |
| B.EA1 | 94 | 94 | 65 | 7 | 0 | — | — | — | — |
| Tok13B | — | — | 100 | 65 | 33 | 8 | 0 | — | — |
| Tok7B | — | — | — | 105 | 87 | 12 | 0 | — | — |
| RT69B | — | — | 100 | 84 | 69 | 37 | 0 | — | — |
| Dth | — | — | — | — | 100 | 93 | 88 | 21 | 1 |
| RT41A | — | — | — | — | 100 | 87 | 92 | 87 | 12 |

^{a}The results of a single experiment are shown. Similar results were obtained in at least two other experiments.
^{b}DNA polymerases were heated and activity was determined using the DNA-directed DNA polymerase unit activity assay as described in Materials and Methods. A reference sample of each DNA polymerase was kept on wet ice and assayed to establish 100% activity.
^{c}— is not determined.

TABLE 39
DNA polymerase specific activities of purified DNA polymerases
with a DNA-DNA and RNA-RNA template-primer^{a}

| | | Specific Activity (Units/mg) | | |
|---|---|---|---|---|
| | Temperature | | | Ratio |
| Enzyme | (° C.)^{b} | DNA-DNA^{c} | RNA-DNA^{d} | (RNA/DNA) |
| Taq | 72 | 80,000 | <1 | — |
| RT41A | 72 | 84,500 | <1 | — |
| Dth | 72 | 37,800 | <1 | — |
| RTth | 72 | 150,000^{e} | 130 | 0.001 |
| Tok7B | 72 | 33,800 | 60 | 0.002 |
| Cth-His | 55 | 12,700 | 30 | 0.002 |
| RT69B | 72 | 16,800 | 50 | 0.003 |
| Tok13B | 72 | 34,800 | 160 | 0.005 |
| Tne | 72 | 31,300 | 325 | 0.01 |
| B.EA1 | 55 | 45,800 | 4,400 | 0.10 |
| Cst-His | 55 | 19,500 | 2,100 | 0.11 |
| CA2 | 55 | 20,000 | 4,900 | 0.25 |

^{a}The results of a single experiment are shown. Similar results were obtained in at least one other experiment.
^{b}Assays were carried out at optimal temperatures.
^{c}Activity with DNA-DNA was determined with activated salmon testes DNA (Materials and Methods)
^{d}Activity with RNA-DNA was determined with CAT cRNA•DNA 20-mer (Materials and Methods)
^{e}Taken from Abramson (1995).

TABLE 40
Catalytic constants of purified DNA polymerases
with DNA-DNA and RNA-DNA template-primer^{a}

| | | k_{CAT} (sec⁻¹) | | |
|---|---|---|---|---|
| | Temperature | | | Ratio |
| Enzyme | (° C.)^{b} | DNA-DNA^{c} | RNA-DNA^{d} | (RNA/DNA) |
| RT41A | 72 | 187 ± 7 | <1 | — |
| Dth | 72 | 39 ± 15 | <1 | — |
| Tne | 72 | 130 ± 32 | 0.2 ± 0.04 | 0.002 |
| RT69B | 72 | 20 ± 5 | 0.6 ± 0.36 | 0.03 |
| Tok13B | 72 | 43 ± 9 | 1.2 ± 0.5 | 0.03 |
| Cth-His | 55 | 28 ± 1 | 16 ± 1 | 0.57 |
| B.EA1 | 55 | 73 ± 16 | 43 ± 7 | 0.59 |
| CA2 | 55 | 82 ± 9 | 48 ± 9 | 0.59 |
| Cst-His | 55 | 40 ± 13 | 88 ± 5 | 2.2 |
| SS II RT | 37 | 16 ± 2 | 45 ± 18 | 2.8 |

^{a}The mean ± standard deviation of two to four determinations is shown.
^{b}Assays were carried out at optimal temperatures.
^{c}Catalytic constants were determined with (dA)₂₇₀•(dT)₄₀ with the exception that for Tne DNA polymerase and SS II RT (dC)ₙ•(dG)₃₅ was used (Materials and Methods).
^{d}Catalytic constants were determined with (rA)₂₅₀•(dT)₃₀ at 55° C. or (rA)₂₅₀•(dT)₄₀ at 72° C.

TABLE 41
Conservative Amino Acid Substitutions

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

1-122. (canceled)

123. An isolated nucleic acid comprising a nucleotide sequence encoding a polypeptide comprising an amino acid sequence that is at least 80% identical to at least forty contiguous amino acids in any one of SEQ ID NOS: 14-25, wherein the polypeptide has a nucleotide polymerase activity.

124. The nucleic acid according to claim 123, wherein the polypeptide encoded by the nucleic acid has both a DNA-dependent and an RNA-dependent nucleotide polymerase activity.

125. A polypeptide comprising an amino acid sequence that is at least 80% identical to at least forty contiguous amino acids in any one of SEQ ID NOS: 14-25 and mutants, fragments and fragments of mutants thereof wherein the polypeptide, mutant, fragment or fragment of mutant has a nucleotide polymerase activity.

126. The polypeptide according to claim 125, wherein the polypeptide has both a DNA-dependent and an RNA-dependent nucleotide polymerase activity.

127. A method of amplifying a double-stranded DNA molecule comprising:
(a) providing a first and second primer, wherein the first primer is complementary to a sequence of the first strand of the DNA molecule and the second primer is complementary to a sequence of the second strand of the DNA molecule;
(b) hybridizing the first primer to the first strand and the second primer to the second strand in the presence of a polypeptide according to claim 6, under conditions such that a third strand of the DNA molecule complementary to the first strand and a fourth strand of the DNA molecule complementary to the second strand are synthesized;
(c) denaturing the first and third strand, the second and fourth strands; and
(d) repeating steps (a) to (c) one or more times.

128. The method according to claim 127, wherein the polypeptide has at least one mutation selected from the group consisting of (1) a mutation that reduces, substantially reduces or eliminates 5′-3′ exonuclease activity of the DNA polymerase, (2) a mutation that results in the DNA polymerase becoming non-discriminating against dideoxynucleotides, and (3) a mutation that increases thermostability of an activity of the polypeptide.

129. A kit for amplifying a DNA molecule, comprising a first container containing a polypeptide according to claim 125.

130. The kit according to claim 129, further comprising a second container containing one or more deoxyribonucleoside triphosphates.

131. A kit according to claim 129, wherein the polypeptide has at least one mutation selected from the group consisting of (1) a mutation that reduces, substantially reduces or eliminates 5′-3′ exonuclease activity of the DNA polymerase, (2) a mutation that results in the DNA polymerase becoming non-discriminating against dideoxynucleotides, and (3) a mutation that increases thermostability of an activity of the polypeptide.

132. The kit according to claim 129, wherein the deoxyribonucleoside triphosphates are selected from the group consisting of: DATP, dCTP, dGTP, dTTP, dITP, 7-deaza-dGTP, dUTP, [α-S]dATP, [α-S]dTTP, [α-S]dGTP, and [α-S]dCTP.

133. The kit according to claim 129, wherein the kit further comprises a container containing a pyrophosphatase.

134. The polypeptide according to claim 125, wherein the polypeptide comprises DNA-dependent DNA polymerase activity, wherein the activity occurs in the presence of magnesium, manganese or a mixture of magnesium and manganese.

135. The polypeptide according to claim 134, wherein the activity occurs in the absence of manganese.

136. The polypeptide according to claim 135, wherein the activity occurs in the absence of manganese.
